SEQUENCE LISTING (6 sequences)

SEQ ID NO: 1 - DNA, 729 bp, Artificial
  Synthetic - sequence synthesised in laboratory
ggatcctcattccggtttttccgtgacggtaaacgttttggttgcatcgtcataggtccattcaccatcaacgccgttgtcattggcgtactgtttgaacactttttcggccgtggcggcatccacggcttcggtcgtggtttcacctttcagcgttttgccgttcagaatcagtttgtacgtgtcggtcttgctgccaccgccaccactgccaccgccagaaccgccaccgctctcaggcttctccgtcacggtaaacgttttggtcgcatcatcgtacgtccattcaccgtcaacgccgttgtcatttgcgtactgtttaaacactttttccgcggtcgctgcatccactgcttccgtggtcgtttcgcctttcagggttttaccattcagaatcagtttgtaggtatccgttttagagccaccgccaccactgccaccgccagaaccgccaccgctttccggtttttcggtaaccgtgaaggttttcgtggcatcatcgtaggtccattcgccatccacaccattatcgttcgcgtactgtttgaaaactttttctgcggttgctgcatccactgcttcggtcgtggtttcgcctttcagcgttttaccgttcaggatcagtttataggtatccgttttgctgccaccgccaccactgccaccgccagaaccgccaccgctgccaccgccaccgccacgtttatcaataaccgccagcacctcgag

SEQ ID NO: 2 - protein, 28 aa, Artificial
  Synthetic - sequence synthesised in laboratory
LEVLAVIDKRGGGGGSGGGSGGGSGGGG

SEQ ID NO: 3 - protein, 14 aa, Artificial
  Synthetic - sequence synthesised in laboratory
SGGGSGGGSGGGGS

SEQ ID NO: 4 - DNA, 7075 bp, Artificial
  Synthetic - sequence synthesised in laboratory
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatcctcattccggtttttccgtgacggtaaacgttttggttgcatcgtcataggtccattcaccatcaacgccgttgtcattggcgtactgtttgaacactttttcggccgtggcggcatccacggcttcggtcgtggtttcacctttcagcgttttgccgttcagaatcagtttgtacgtgtcggtcttgctgccaccgccaccactgccaccgccagaaccgccaccgctctcaggcttctccgtcacggtaaacgttttggtcgcatcatcgtacgtccattcaccgtcaacgccgttgtcatttgcgtactgtttaaacactttttccgcggtcgctgcatccactgcttccgtggtcgtttcgcctttcagggttttaccattcagaatcagtttgtaggtatccgttttagagccaccgccaccactgccaccgccagaaccgccaccgctttccggtttttcggtaaccgtgaaggttttcgtggcatcatcgtaggtccattcgccatccacaccattatcgttcgcgtactgtttgaaaactttttctgcggttgctgcatccactgcttcggtcgtggtttcgcctttcagcgttttaccgttcaggatcagtttataggtatccgttttgctgccaccgccaccactgccaccgccagaaccgccaccgctgccaccgccaccgccacgtttatcaataaccgccagcacctcgagtgccttggctttgacgtatcgcccaggcgcgggttcgattgcgcgatagcgcgcattgccatagttggcgggcgcggcgcgtttcgcgccggcctgcccggccagccatgcggtccagtccggccaccagctgccgtgatgctcgatggcgccggccagccattgctgcggcgactccggcagcgcatcgttagtccagtggctgcgcttgttcttggccggcgggttgatcacaccggcgatatggcccgacgcacccagcacgaagcgcagcttgttcgccagcagcgcggtcgaggcataggccgcggtccacggcacgatatggtcttcgcgcgagccgtagatataggtcggcacgtcgatgctggccaggtccaccggcacgccgcacacggtcagcttgcccggtaccttgagctcgttctgcaggtaggtgtggcgcaggtaccagcagtaccacggccccggcaggttggtggcgtcgccgttccagaacagcaggtcgaacggcaccggcgtgttgcccttcaggtagttgtcgaccacgtagttccacaccaggtcgttcgggcgcaagaacgagaaggtattggccagctcaaggccgcgcagcagcgcgcacggcgcgccggcgccgccgcccagcgtggcctcgcgcaactgcacatggccctcgtcgacaaagacgtcgaggatgcccgtgtcggcaaagtccagcagcgtggtcagcagcgtgacgctggcggccgggtgctcgccgcgcgcggccagcaccgccagcgcggtcgagacaatggtgccgcccacgcagaagccgagcacgttgatcttgtcctggccgctgatgtcgcgcgcgacttcgatggcgcggatggccgcgtgctcgatgtagtcgtcccaggtgctgccggccatgctggcgtccggattgcgccacgacaccagaaacaccgtatgtccctgctccaccacatggcgcaccagcgagctctccggctgcaggtccaggatgtagtacttgttgatgcacggcggcaccatcagcagcgggcgcgcgtgcaccttgtcggtcagcggcttgtactgcaacagctggaagtactcgttctcgaagaccacggcgccttcggtcaccgcgacattgcggccgacctcaaacgcgctctcgtcggtctgcgagatcttgccgcgtgtcaggtcttccatcatgttgcgcacgccggcacgcagcgattcgccgcccgactcgatcagcaggcgctgcgcctcgggattggtggcaaggaagttggcgggcgacatcgcatcgacccattgcgagatcgcgaagcggatgcgctggcgggtcttggcatcggcctcgacggcatcggccagctcggtcaaggcgcgcgcattgagcaggtagaacgcggcagcgaagcgatatgggaggttggtgcgccatgcgtcgccggcgaagcgccggtcgtgcagcggaccggtggcctcggccttgccctcggccatggcctgccacagcgctgagaagtccttcatgtagcgctgctggatatcacccagctgcgccggcgcgatcttgacgcctgccagcgcatccaggcccggaatgccggacgcggccgcgtggccgttgccttcagtgccctgccactggcgggaccattccagccatgtggctggatcgaatggccccggcgtgaccttgaatggttgggacttgccttcctgcgtggaagctgccgcgcctttgccggtcgccatactagtatctccttatttctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 5 - DNA, 36 bp, Artificial
  Synthetic PCR primer
ggactctcgagagcatggcccgaccaatcggtacag

SEQ ID NO: 6 - DNA, 36 bp, Artificial
  Synthetic PCR primer
gtacaggatcctcacgacgcccgcacggtcggtgac

(19) United States
(12) Patent Application Publication
(10) Pub. No.: US 2013/0337528 A1
(43) Pub. Date: Dec. 19, 2013

(54) COMPOSITIONS FOR SEPARATION METHODS

(71) Applicants: Tracy Thompson, Palmerston North (NZ); Bernd Helmut Rehm, Palmerston North (NZ); Andrew Brian Herbert, Nelson (NZ); Edward George Saravolac, Palmerston Nolk (NZ)

(72) Inventors: Tracy Thompson, Palmerston North (NZ); Bernd Helmut Rehm, Palmerston North (NZ); Andrew Brian Herbert, Nelson (NZ); Edward George Saravolac, Palmerston Nolk (NZ)

(21) Appl. No.: 13/992,813

(22) PCT Filed: Dec. 9, 2011
(86) PCT No.: PCT/IB2011/055564

§ 371 Date: Aug. 26, 2013

Related U.S. Application Data
(60) Provisional application No. 61/421,669, filed on Dec. 10, 2010.

(51) Int. Cl. C07K 1/34

(52) U.S. Cl. CPC C07K 1/34
USPC 435/178; 530/389.1; 530/402; 435/272

(57) ABSTRACT

This invention relates generally to the fields of separation and conversion technologies, and more particularly to materials for use in tangential-flow filtration techniques. The tangential-flow materials are useful in a wide range of separation and conversion processes, including those reliant on reverse osmosis, microfiltration, ultrafiltration, or nanofiltration semipermeable filtration membranes, and provide efficient methods for purifying or producing various target substances, including biopolymer particles for use in tangential-flow filtration.

### TECHNICAL FIELD

This invention relates generally to the fields of separation and conversion technologies, and more particularly to materials for use in tangential-flow filtration techniques. The tangential-flow materials are useful in a wide range of separation and conversion processes, including those reliant on reverse osmosis, microfiltration, ultrafiltration, or nanofiltration semipermeable filtration membranes, and provide efficient methods for purifying or producing various target substances.

### BACKGROUND OF THE INVENTION

The separation of desirable target substances from undesirable substances, for example from a complex composition, is a fundamental step in the production of many important commodities, including foods, chemicals, pharmaceuticals, and biologics such as cells, viruses, polypeptides, polynucleotides, and metabolites. Similarly, the conversion of one or more precursor substances into a target substance, for example by enzymatic conversion, optionally coupled with enrichment or separation of the target substance, for example from the precursor substance(s), is a fundamental step in many methods of manufacture.

As a consequence, there has been a great deal of expenditure to develop methods and technologies to enable the efficient separation and production of desirable target substances. For example, reverse osmosis (RO), microfiltration (MF), ultrafiltration (UF), and nanofiltration (NF) techniques for the separation of one or more desirable substances, typically one or more liquids, from a second component, such as another liquid or one or more dissolved or suspended solids, have been developed over many years.

Most commonly used is packed bed chromtography where resin particles are packed into a bed and a solution containing the target molecule is passed through the column and the target binds to the resins. A particular challenge of this method is the formation of irregular flow channels. These irregular flow channels prevent the efficient purification of the target as well as preventing efficient cleaning of the resin, thereby creating a potential for contamination.

In the production of monoclonal antibodies, for example, it has been suggested that packing the bed with ten-fold excess resin could address the issue of irregular flow path. Clearly, there are cost and efficiency consequences in adopting such a suggestion. In a system with access to all the available media it would be possible to reduce the amount of resin required, thus reducing the cost of production.

One solution to reducing the risk of irregular flow path is to reduce the operating pressure of the column. While this has the desired effect of reducing the risk of channelling, the negative consequence of an increase in processing times.

Tangential-flow (also referred to as cross-flow) filtration processes, where the flow is across the surface of a semipermeable membrane surface, and a concentrate stream is typically withdrawn downstream of the feed flow path, has the potential to address some of these issues. A variety of semipermeable membranes have been developed for use in these and other filtration processes.

Existing tangential-flow technologies, while satisfactory in achieving particular separations, are not well suited to application in the preparation or separation of certain target substances, such as polypeptides, from complex feedstocks. Source liquids having a high level of particulates or filtration technologies utilising particles are generally ill-suited to application in tangential-flow filtration, typically as they may form gel-layers or otherwise block or stick to the filter membrane, thereby reducing efficiency. Attempts to solve this problem have focussed on the use of large rigid particles (in the order of 100 to 300 microns). While the large size and rigidity reduces the risk or degree of fouling, the reduced surface area to volume ratio presents fewer binding sites to the target requiring the use of more resin.

It is an object of the present invention to overcome or at least ameliorate some of the above disadvantages, to provide improved compositions and methods for the preparation and purification of target substances, in particular by tangential-flow filtration, or at least to provide the public with a useful choice.

Other objects of the invention may become apparent from the following description which is given by way of example only.

### SUMMARY OF THE INVENTION

The present invention relates to a method for preparing one or more target substances from a source material, the method comprising contacting the source material with a population of amorphous polymer particles for a time sufficient to allow the amorphous polymer particles to bind one or more target substances or one or more precursors of a target substance or one or more contaminants, separating by tangential-flow filtration the one or more contaminants from the particle-bound target substance or precursor thereof or the one or more target substances or precursor thereof from a particle-bound contaminant, and recovering the target substance.

In one embodiment, the population of amorphous polymer particles is a homogeneous population. In another embodiment, the population of amorphous polymer particles is a heterogeneous population.

In one embodiment, one or more of the amorphous polymer particles comprises one or more biopolymers selected from a polyester, polyester, polythioester or a polyhydroxyalkanoate.

In one embodiment, one or more of the amorphous polymer particles is or is capable of being synthesised by a particle-forming protein. In one embodiment, substantially all of the population of polymer particles is or is capable of being synthesised by a particle-forming protein.

In one embodiment, one or more of the amorphous polymer particles comprises a polymer particle-forming protein, such as a polymer synthase or a polymer synthase fusion.

In one embodiment, the recovery of the target substance is by elution from the polymer particle. In one embodiment, the recovery of the target substance is by collection of the tangential-flow filtration permeate. In one embodiment, the recovery of the target system is by collection of the tangential-flow filtration retentate.

Accordingly, in one aspect, the present invention provides a method for separating or purifying one or more target substances from a source material, the method comprising contacting the source material with a population of polymer particles for a time sufficient to allow one or more of the polymer particles to bind one or more target substances, separating one or more contaminants from the particle-bound target substance by tangential-flow filtration, and recovering the target substance, wherein one or more of the polymer particles comprises:
a biopolymer selected from a polyester, a polythioester or a polyhydroxyalkanoate; or
a polymer particle-forming polypeptide, such as a polymer synthase or a polymer synthase fusion; or
both of the above.

Accordingly, in another aspect, the present invention provides a method for separating or purifying one or more target substances from a source material, the method comprising contacting the source material with a population of polymer particles for a time sufficient to allow one or more of the polymer particles to bind one or more contaminants, separating one or more target substances from the particle-bound contaminants by tangential-flow filtration, and recovering the target substance, wherein one or more of the polymer particles comprises:
a biopolymer selected from a polyester, polyester, polythioester or a polyhydroxyalkanoate; or
a polymer particle-forming polypeptide, such as a polymer synthase or a polymer synthase fusion; or
both of the above.

In another aspect, the present invention provides a method for preparing one or more reaction products, the method comprising contacting by tangential-flow filtration a source material comprising one or more reaction substrates with one or more polymer particles for a sufficient time to allow the one or more polymer particles to bind a desired fraction of the one or more reaction substrates, optionally separating one or more contaminants from the polymer particles by tangential-flow filtration, and recovering the reaction product, wherein the one or more polymer particles comprise a catalyst of the reaction, and wherein one or more of the polymer particles comprises:
a biopolymer selected from a polyester, polyester, polythioester or a polyhydroxyalkanoate; or
a polymer particle-forming polypeptide, such as a polymer synthase or a polymer synthase fusion; or
both of the above.

The invention further relates to a purification method for separating a target substance from a source material, the method comprising (a) providing a source material, (b) tangential-flow filtering said source material with at least one semipermeable filter wherein the source material or the semipermeable filter comprises one or more polymer particles, and (c) recovering the target substance, wherein one or more of the source material, the semipermeable filter, or one or more solutions used in said tangential flow filtering comprises one or more polymer particles, wherein the one or more polymer particles comprise a ligand capable of binding the target substance, and wherein one or more of the polymer particles comprises:
i. a biopolymer selected from a polyester, a polythioester or a polyhydroxyalkanoate; or
ii. a polymer particle-forming polypeptide, such as a polymer synthase or a polymer synthase fusion; or
iii. a polymer particle-binding polypeptide;
iv. a polypeptide fusion partner;
v. an affinity ligand;
vi. an enzyme;
vii. a fusion polypeptide comprising two or more of the above; or
viii. any combination of any two or more of (i) to (vii) above.

Accordingly, in one exemplary embodiment the invention provides a purification method for purifying one or more antibodies, which comprises providing a source material comprising one or more antibodies, tangential-flow filtering said source material with at least one semipermeable filter comprising one or more polymer particles, wherein the one or more polymer particles comprise a ligand capable of binding an antibody, and recovering the antibody.

Accordingly, in another exemplary embodiment the invention provides a purification method for purifying one or more polymer particles, which comprises providing a source material comprising one or more polymer particles, tangential-flow filtering said source material with at least one semi-permeable filter, wherein one or more of the polymer particles comprises:
a biopolymer selected from a polyester, a polythioester or a polyhydroxyalkanoate; or
a polymer particle-forming polypeptide, such as a polymer synthase or a polymer synthase fusion; or
both of the above.

In a further aspect, the present invention provides a polymer particle comprising
a biopolymer selected from a polyester, polythioester or a polyhydroxyalkanoate; or
a polymer particle-forming polypeptide, such as a polymer synthase or a polymer synthase fusion; or
both of the above; and
   wherein one or more of the fusion polypeptides is or comprises the GB1 domain of protein G from Streptococcus spp.

A fusion polypeptide comprising a polymer particle-forming polypeptide and one or more GB1 domain of protein G from Streptococcus spp.

In one embodiment, the fusion polypeptide is or comprises a GB1 domain encoded by a polynucleotide sequence comprising 12 or more contiguous nucleotides of SEQ ID NO. 4. In another embodiment, the fusion polypeptide is or comprises a polypeptide encoded by a polynucleotide sequence comprising 12 or more contiguous nucleotides of SEQ ID NO. 4.

In one embodiment, said polymer particle has a immunoglobulin binding capacity of greater than 30 mg immunoglobulin/g wet polymer particle.

In one embodiment, the binding capacity is at least about 35 mg immunoglobulin/g wet polymer particle, about 40 mg immunoglobulin/g wet polymer particle, about 45 mg immunoglobulin/g wet polymer particle, about 50 mg immunoglobulin/g wet polymer particle, about 55 mg immunoglobulin/g wet polymer particle, or about 60 mg immunoglobulin/g wet polymer particle.

In one embodiment, the immunoglobulin is IgG.

In a further aspect, the invention provides a method for making a semipermeable filter for use in tangential-flow filtration, the method comprising providing a permeable or semipermeable support, associating one or more polymer particles with the support to provide a semipermeable filter, wherein one or more of the polymer particles comprises:
a biopolymer selected from a polyester, polythioester or a polyhydroxyalkanoate; or
a polymer particle-forming polypeptide, such as a polymer synthase or a polymer synthase fusion; or
both of the above.

In a further aspect, the invention provides a method for preparing polymer particles, wherein one or more of the polymer particles comprises:
a biopolymer selected from a polyester, polythioester or a polyhydroxyalkanoate; or
a polymer particle-forming polypeptide, such as a polymer synthase or a polymer synthase fusion; or
both of the above;
   wherein the method comprises separating by tangential-flow filtration one or more contaminants from the polymer particles, and recovering the polymer particles.

In another aspect the invention provides a method for separating or purifying one or more polymer particles from a source material, the method comprising separating one or more contaminants from the polymer particles by tangential-flow filtration, and recovering the one or more polymer particles, wherein one or more of the particles comprises
a biopolymer selected from a polyester, polythioester or a polyhydroxyalkanoate; or
a polymer particle-forming polypeptide, such as a polymer synthase or a polymer synthase fusion; or
both of the above.

The invention further provides compositions, membranes, filters, and filter apparatuses (such as filter cartridges), comprising one or more polymer particles as described herein. Such compositions, membranes, filters, and filter apparatuses are particularly suitable for use in tangential-flow filtration.

The following embodiments may relate to any of the above aspects.

In various embodiments, one or more of the polymer particles comprises one or more of the following:
a polymer particle-binding polypeptide;
a polypeptide fusion partner;
an affinity ligand;
an enzyme;
a fusion polypeptide comprising two or more of the above; or
any combination of any two or more of the above.

In various embodiments, substantially all of the polymer particles comprise:
a biopolymer, such as a biopolymer selected from poly-beta-hydroxy acids, biopolylactates, biopolythioesters, and biopolyesters; or
a polymer particle-forming polypeptide, such as a polymer synthase or a polymer synthase fusion; or
both of the above.

In one embodiment, the polymer particle-forming polypeptide is covalently bound to the surface of the particle.

In one embodiment, the one or more polymer particles comprises one or more ligands displayed on the surface thereof.

In one embodiment, the polymer particles are bound to, associated with or comprise a semipermeable support, such as a semipermeable membrane, resin, tangential-flow filter, tangential-flow filter cartridge, or the like.

In one embodiment, the source material is or is derived from a cell lysate. In one embodiment, the source material is or is derived from a protein expression system, including an in vitro protein expression system.

In one embodiment, the source material is or is derived from a food, including a dairy product or dairy processing stream, a fermentate including a wine or beer fermentate, and the like.

In one embodiment, the source material is a solution, including a reaction solution, a chemical synthesis solution, a chemical synthesis intermediate, and the like.

In one embodiment, the target substance is a polypeptide, including for example, a recombinant polypeptide, an antibody, an enzyme, a hormone, and the like.

In one embodiment, the target substance is a polynucleotide, including for example, a recombinant polynucleotide, a vector, an oligonucleotide, an RNA molecule such as an rRNA, an mRNA, an miRNA, an siRNA, or a tRNA, or a DNA molecule such as a cDNA.

In one embodiment, the target substance is a cellular metabolite, including a secreted metabolite.

In various embodiments, the polymer particle comprises a biopolymer selected from a polyester, polythioester or a polyhydroxyalkanoate (PHA). Most preferably the polymer comprises polyhydroxyalkanoate, preferably poly(3-hydroxybutyrate) (PHB).

In various embodiments, the polymer constituting the particle consists essentially of, or consists a biopolymer selected from a polyester, polythioester or a polyhydroxyalkanoate (PHA). Most preferably the polymer comprises polyhydroxyalkanoate, preferably poly(3-hydroxybutyrate) (PHB).

In various embodiments the polymer particle comprises a polymer particle encapsulated by a phospholipid monolayer.

In various embodiments the polymer synthase is bound to the polymer particle or to the phospholipid monolayer or is bound to both.

In various embodiments the polymer particle comprises two or more different fusion polypeptides.

In various embodiments the polymer particle comprises two or more different fusion polypeptides on the polymer particle surface.

In various embodiments the polymer particle comprises three or more different fusion polypeptides, such as three or more different fusion polypeptides on the polymer particle surface.

In various embodiments the polymer particle further comprises at least one substance bound to or incorporated into the polymer particle, or a combination thereof.

In various embodiments the substance is bound to the polymer particle by cross-linking

In various embodiments the polymer synthase is bound to the polymer particle or to the phospholipid monolayer or is bound to both.

In various embodiments the polymer synthase is covalently or non-covalently bound to the polymer particle it forms.

In various embodiments the polymer synthase is a PHA synthase from the class 1 genera Acinetobacter, Vibrio, Aeromonas, Chromobacterium, Pseudomonas, Zoogloea, Alcaligenes, Delftia, Burkholderia, Ralstonia, Rhodococcus, Gordonia, Rhodobacter, Paracoccus, Rickettsia, Caulobacter, Methylobacterium, Azorhizobium, Agrobacterium, Rhizobium, Sinorhizobium, Rickettsia, Crenarchaeota, Synechogstis, Ectothiorhodospira, Thiocapsa, Thyogstis and Allochromatium, the class 2 genera Burkholderia and Pseudomonas, or the class 4 genera Bacillus, more preferably from the group comprising class 1 Acinetobacter sp. RA3849, Vibrio cholerae, Vibrio parahaemolyticus, Aeromonas punctata FA440, Aeromonas hydrophila, Chromobacterium violaceum, Pseudomonas sp. 61-3, Zoogloea ramigera, Alcaligenes latus, Alcaligenes sp. SH-69, Delftia acidovorans, Burkholderia sp. DSMZ9242, Ralstonia eutrophia H16, Burkholderia cepacia, Rhodococcus rubber PP2, Gordonia rubripertinctus, Rickettsia prowazekii, Synechogstis sp. PCC6803, Ectothiorhodospira shaposhnikovii N1, Thiocapsa pfennigii 9111, Allochromatium vinosum D, Thyogstis violacea 2311, Rhodobacter sphaeroides, Paracoccus denitrificans, Rhodobacter capsulatus, Caulobacter crescentus, Methylobacterium extorquens, Azorhizobium caulinodans, Agrobacterium tumefaciens, Sinorhizobium meliloti 41, Rhodospirillum rubrum HA, and Rhodospiriullum rubrum ATCC25903, class 2 Burkholderia caryophylli, Pseudomonas chloraphis, Pseudomonas sp. 61-3, Pseudomonas putida U, Pseudomonas oleovorans, Pseudomonas aeruginosa, Pseudomonas resinovorans, Pseudomonas stutzeri, Pseudomonas mendocina, Pseudomonas pseudolcaligenes, Pseudomonas putida BM01, Pseudomonas nitroreducins, Pseudomonas chloraphis, and class 4 Bacillus megaterium and Bacillus sp. INT005.

In other embodiments the polymer synthase is a PHA polymer synthase from Gram-negative and Gram-positive eubacteria, or from archaea.

In various examples, the polymer synthase may comprise a PHA polymer synthase from C. necator, P. aeruginosa, A. vinosum, B. megaterium, H. marismortui, P. aureofaciens, or P. putida, which have Accession No.s AY836680, AE004091, AB205104, AF109909, YP137339, AB049413 and AF150670, respectively.

Other polymer synthases amenable to use in the present invention include polymer synthases, each identified by it accession number, from the following organisms: R. eutropha (A34341), T. pfennigii (X93599), A. punctata (O32472), Pseudomonas sp. 61-3 (AB014757 and AB014758), R. sphaeroides (AAA72004), C. violaceum (AAC69615), A. borkumensis SK2 (CAL17662), A. borkumensis SK2 (CAL16866), R. sphaeroides KD131 (ACM01571 and YP002526072), R. opacus B4 (BAH51880 and YP002780825), B. multivorans ATCC 17616 (YP001946215 and BAG43679), A. borkumensis SK2(YP693934 and YP693138), R. rubrum (AAD53179), gamma proteobacterium HTCC5015 (ZP05061661 and EDY86606), Aoarcus sp. BH72 (YP932525), C. violaceum ATCC 12472 (NP902459), Limnobacter sp. MED105 (ZP01915838 and EDM82867), M. algicola DG893 (ZP01895922 and EDM46004), R. sphaeroides (CAA65833), C. violaceum ATCC 12472 (AAQ60457), A. latus (AAD10274, AAD01209 and AAC83658), S. maltophilia K279a (CAQ46418 and YP001972712), R. solanacearum IPO1609 (CAQ59975 and YP002258080), B. multivorans ATCC 17616 (YP001941448 and BAG47458), Pseudomonas sp. gl13 (ACJ02400), Pseudomonas sp. gl06 (ACJ02399), Pseudomonas sp. gl01 (ACJ02398), R. sp. gl32 (ACJ02397), R. leguminosarum bv. viciae 3841 (CAK10329 and YP770390), Aoarcus sp. BH72 (CAL93638), Pseudomonas sp. LDC-5 (AAV36510), L. nitroferrum 2002 (ZP03698179), Thauera sp. MZ1T (YP002890098 and ACR01721), M. radiotolerans JCM 2831 (YP001755078 and ACB24395), Methylobacterium sp. 4-46 (YP001767769 and ACA15335), L. nitroferrum 2002 (EEG08921), P. denitrificans (BAA77257), M. gryphiswaldense (ABG23018), Pseudomonas sp. USM4-55 (ABX64435 and ABX64434), A. hydrophila (AAT77261 and AAT77258), Bacillus sp. INT005 (BAC45232 and BAC45230), P. putida (AAM63409 and AAM63407), G. rubripertinctus (AAB94058), B. megaterium (AAD05260), D. acidovorans (BAA33155), P. seriniphilus (ACM68662), Pseudomonas sp. 14-3 (CAK18904), Pseudomonas sp. LDC-5 (AAX18690), Pseudomonas sp. PC17 (ABV25706), Pseudomonas sp. 3Y2 (AAV35431, AAV35429 and AAV35426), P. mendocina (AAM10546 and AAM10544), P. nitroreducens (AAK19608), P. pseudoalcaligenes (AAK19605), P. resinovorans (AAD26367 and AAD26365), Pseudomonas sp. USM7-7 (ACM90523 and ACM90522), P. fluorescens (AAP58480) and other uncultured bacterium (BAE02881, BAE02880, BAE02879, BAE02878, BAE02877, BAE02876, BAE02875, BAE02874, BAE02873, BAE02872, BAE02871, BAE02870, BAE02869, BAE02868, BAE02867, BAE0286, BAE02865, BAE02864, BAE02863, BAE02862, BAE02861, BAE02860, BAE02859, BAE02858, BAE02857, BAE07146, BAE07145, BAE07144, BAE07143, BAE07142, BAE07141, BAE07140, BAE07139, BAE07138, BAE07137, BAE07136, BAE07135, BAE07134, BAE07133, BAE07132, BAE07131, BAE07130, BAE07129 BAE07128, BAE07127, BAE07126, BAE07125, BAE07124, BAE07123, BAE07122, BAE07121 BAE07120, BAE07119, BAE07118, BAE07117, BAE07116, BAE07115, BAE07114, BAE07113 BAE07112, BAE07111, BAE07110, BAE07109, BAE07108, BAE07107, BAE07106, BAE07105, BAE07104, BAE07103, BAE07102, BAE07101, BAE07100, BAE07099, BAE07098, BAE07097, BAE07096, BAE07095, BAE07094, BAE07093, BAE07092, BAE07091, BAE07090, BAE07089, BAE07088, BAE07053, BAE07052, BAE07051, BAE07050, BAE07049, BAE07048, BAE07047, BAE07046, BAE07045, BAE07044, BAE07043, BAE07042, BAE07041, BAE07040, BAE07039, BAE07038, BAE07037, BAE07036, BAE07035, BAE07034, BAE07033, BAE07032, BAE07031, BAE07030, BAE07029, BAE07028, BAE07027, BAE07026, BAE07025, BAE07024, BAE07023, BAE07022, BAE07021, BAE07020, BAE07019, BAE07018, BAE07017, BAE07016, BAE07015, BAE07014, BAE07013, BAE07012, BAE07011, BAE07010, BAE07009, BAE07008, BAE07007, BAE07006, BAE07005, BAE07004, BAE07003, BAE07002, BAE07001, BAE07000, BAE06999, BAE06998, BAE06997, BAE06996, BAE06995, BAE06994, BAE06993, BAE06992, BAE06991, BAE06990, BAE06989, BAE06988, BAE06987, BAE06986, BAE06985, BAE06984, BAE06983, BAE06982, BAE06981, BAE06980, BAE06979, BAE06978, BAE06977, BAE06976, BAE06975, BAE06974, BAE06973, BAE06972, BAE06971, BAE06970, BAE06969, BAE06968, BAE06967, BAE06966, BAE06965, BAE06964, BAE06963, BAE06962, BAE06961, BAE06960, BAE06959, BAE06958, BAE06957, BAE06956, BAE06955, BAE06954, BAE06953, BAE06952, BAE06951, BAE06950, BAE06949, BAE06948, BAE06947, BAE06946, BAE06945, BAE06944, BAE06943, BAE06942, BAE06941, BAE06940, BAE06939, BAE06938, BAE06937, BAE06936, BAE06935, BAE06934, BAE06933, BAE06932, BAE06931, BAE06930, BAE06929, BAE06928, BAE06927, BAE06926, BAE06925, BAE06924, BAE06923, BAE06922, BAE06921, BAE06920, BAE06919, BAE06918, BAE06917, BAE06916, BAE06915, BAE06914, BAE06913, BAE06912, BAE06911, BAE06910, BAE06909, BAE06908, BAE06907, BAE06906, BAE06905, BAE06904, BAE06903, BAE06902, BAE06901, BAE06900, BAE06899, BAE06898, BAE06897, BAE06896, BAE06895, BAE06894, BAE06893, BAE06892, BAE06891, BAE06890, BAE06889, BAE06888, BAE06887, BAE06886, BAE06885, BAE06884, BAE06883, BAE06882, BAE06881, BAE06880, BAE06879, BAE06878, BAE06877, BAE06876, BAE06875, BAE06874, BAE06873, BAE06872, BAE06871, BAE06870, BAE06869, BAE06868, BAE06867, BAE06866, BAE06865, BAE06864, BAE06863, BAE06862, BAE06861, BAE06860, BAE06859, BAE06858, BAE06857, BAE06856, BAE06855, BAE06854, BAE06853 and BAE06852).

In various embodiments the polymer synthase can be used for the in vitro production of polymer particles by polymerising or facilitating the polymerisation of the substrates (R)-Hydroxyacyl-CoA or other CoA thioester or derivatives thereof.

In various embodiments the substrate or the substrate mixture comprises at least one optionally substituted amino acid, lactate, ester or saturated or unsaturated fatty acid, preferably acetyl-CoA.

In various embodiments, the catalyst is an enzyme. In a representative example, the catalyst is an enzyme, the precursor substance is a substrate of the enzyme, and the target substance is a product of the reaction catalysed by the enzyme. In further embodiments, the population of polymer particles may comprise more than one or more enzyme. Particularly contemplated are embodiments wherein the population of polymer particles comprises two or more enzymes wherein the product of a reaction catalysed by another enzyme, such as, for example, two or more enzymes comprising part or all of a synthetic or catalytic pathway.

In one embodiment, the one or more polymer particles are permanently associated with the permeable or semipermeable support. In another embodiment, the one or more polymer particles are reversibly associated with the permeable or semipermeable support.

In various embodiments the one or more polymer particles are covalently or non-covalently bound to the semipermeable filter. For example, the one or more polymer particles are adsorbed onto a semipermeable support or membrane. In another example, the one or more polymer particles comprise a ligand capable of binding to the semipermeable support or membrane.

In various embodiments, the semipermeable support comprises one or more of the following: polyethersulfone, PVDF, PP, PEES HDPE (high density polyethylene), PP (polypropylene), PEEK (polyetheretherketone), PET and FEP (fluorinated ethylene propylene). In another embodiment, the semipermeable support comprises a polysaccharide including, for example, cellulose, derivatised cellulose, or stabilised cellulose. In yet another embodiment, the semipermeable support comprises one or more ceramics.

In various embodiments, the semipermeable filter is in one of the following configurations: spirally-wound, plate & frame, flat sheet, hollow fibre, spin-disc, or tubular. Examples thereof may conveniently be provided as a cassette or cartridge.

In various embodiments, the one or more polymer particles are prepared, separated, or purified by tangential-flow filtration in the presence of one or more of the following: a detergent, a pH modifier, one or more solvents, one or more chaotropes, one or more enzymes, and one or more thiols. For example, the tangential-flow filtration includes a chemical treatment such as acid or base treatments. In various embodiments, the method comprises one or more of the chemical treatments exemplified herein, for example, one or more of the treatments exemplified in Example 12.

In various embodiments, the method of preparing, separating, or purifying one or more substances or one or more polymer particles using tangential-flow filtration comprises or is preceded or followed by homogenisation, microfluidization, sonication, centrifugation or any combination thereof.

In various embodiments, the ligand capable of binding an antibody is selected from the group comprising protein A, protein G, protein A/G, protein L, a recombinant variant thereof, a functional fragment thereof including recombinant functional fragments thereof, such as the Z domain of protein A, and any combination thereof, such as a ZZ domain comprising a contiguous repeat of the Z domain of protein A.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

Further aspects and advantages of the present invention will become apparent from the ensuing description which is given by way of example only.

### DESCRIPTION OF THE DRAWINGS

FIG. 1 presents schematic diagrams of tangential-flow filtration (FIG. 1A) and an exemplary simple tangential-flow filtration system (FIG. 1B) utilising a feed pump to allow recirculation of the permeate in the system from a feed reservoir through the tangential-flow filtration membrane cartridge.

FIGS. 2 to 4 show general schema that may be used purify or prepare one or more target substances from a source liquid using the methods of the present invention.

FIG. 5 presents a photograph of an SDS-PAGE analysis of the polymer particle protein profiles (Coomassie blue and silver staining) of the ZZPhaC-polymer particles used in tangential-flow filtration purification of IgG immunoglobulins, as described herein in the Examples. Lane 1, MW marker; Lane 2, unfiltered granules; Lane 3, retentate from 100 kDa filtration; Lane 4, retentate from 0.1 μm filtration; Lane 3, retentate from 0.2 nm filtration.

FIG. 6 presents GC/MS spectra of the polymer particle protein profiles (of the ZZPhaC-polymer particles used in tangential-flow filtration purification of IgG immunoglobulins, as described herein in the Examples.

FIG. 7 presents a transmission electron micrograph of ZZ-polymer particles before (A) and after (B) diafiltration with feedstock, as described herein in the Examples.

FIG. 8 is a graph showing the permeate fraction elution profile from the TFF diafiltration of a mixture of BSA and IgG. A 5 BSA was not bound to the polymer particles of the present invention comprising the Z-domain and was readily removed from the system. Upon concentrating and treating the retentate beads with 50 mM Citrate 150 mM Saline pH 3.0 (at fraction 13) the IgG was released from the beads and readily diafiltered from the retentate.

FIG. 9 depicts SDS-PAGE analysis of permeate fractions from the TFF separation of Human IgG from BSA as described herein in Example 2. FIG. 9A shows elution of BSA containing fractions in the first TFF 280 nm peak (1×PBS pH 7.4 wash fractions). FIG. 9B shows elution of IgG containing fractions after diafiltration of the polymer particles of the present invention comprising the Z-domain beads with citrate (pH 3.0).

FIG. 10 is an elution profile showing the purification of goat IgG from goat serum using polymer particles of the present invention comprising the GB1-domain from protein G and TFF, as described in Example 3. A 50 mL suspension of 1:10 diluted (in PBS) goat serum was incubated with 5 g wet weight polymer particles of the present invention comprising the GB1-domain particles and then diafiltered (50 cm² cartridge, 0.1 um) to remove serum proteins. IgG was eluted after concentration to 20 mL and at fraction 15 diafiltering against 50 mM sodium citrate in 150 mM NaCl, pH 3.0.

FIG. 11 shows SDS-PAGE analysis of TFF permeate fractions of IgG purification from goat serum. FIG. 11A shows the silver stained serum proteins eluted from TFF. FIG. 11B shows the proteins eluted from TFF after diafiltration with citrate-saline at pH 3.0, with the IgG heavy and IgG light chains clearly visible as predominant proteins in the eluted fractions.

FIG. 12 shows a graph of the measurement of the removal of colloidal gold from solution using TFF and polymer particles of the present invention comprising a gold-binding-domain, as described in Example 4. A 30 ml suspension of 0.005% colliodial gold was recirculated in a TFF system with a 20 cm², 0.2 um hollow fiber microfiltration cartridge with a permeate flow of 9 ml/min. At 8, 13 and 29 minutes (*) 30 mg, 300 mg and 300 mg, respectively of polymer particles of the present invention comprising the gold-binding-domain were added to the retentate. The absorbance of the colloidal gold in the permeate was measured at 520 mm

FIG. 13 shows a graph of the recovery of maltose from an amylase-bound particle mediated bioconversion of starch using TFF, as described herein in Example 5. Suspensions of soluble starch (300 ml 1%. 4% and 8% w/v) were converted to maltose using 2 g of PolyEnz-Amy beads. The suspensions were filtered by TFF to recover the maltose in the permeate and contain the beads in the retentate fractions.

FIG. 14 shows the conversion of methy parathion to para-nitrophenol with organophosphate hydrolase-bound particles during TFF, as described in Example 7. A 30 ml solution of methyl parathion (200 uM) was recirculated in a TFF system with a 20 cm², 0.2 um hollow fiber cartridge. Two cycles of the bioconversion under the same conditions were recorded.

FIG. 15 shows the removal of para-nitrophenol from a suspension of organophosphate hydrolase-bound particles using TFF diafiltration, as described herein in Example 7.

FIG. 16 shows an illustration of a simplified small scale crossflow filtration process scheme for the purification of PHB polymer particles taken directly from cell homogenate, as described herein in Example 8. The strategy is designed to allow for a highly disperse homogenate suspension by using extensive microfluidization. After homogenization, DNAase and MgCl₂ are added to reduce the size of DNA fragments prior to filtration. MgCl₂ is added in large excess to the EDTA to allow the DNAase to be active. During TFF the homogenate suspension is diafiltered into eight volumes of diafiltration buffer to remove host cell proteins and nucleic acid.

FIG. 17A depicts permeate elution profile TFF of 250 ml crude cell homogenate as described in Example 9 herein. The TFF purification was performed on the cell homogenate on 110 cm² 0.1 μm hollow fiber cartridge using 8 diafiltration volumes of PBS-EDTA in 20% EtOH. FIG. 17B is a graph of a Bradford protein assay on the permeate fractions from the TFF purification of cell homogenates. FIG. 17C is a SDS-PAGE of permeate fractions. 20 μl aliquots of permeate fractions 1-8 (lanes 3-10) were run on a 15% Gel (lane 1, mw std; lane 2, 20 μl final bead suspension). The samples were loaded by volume and were not adjusted for protein content.

FIG. 18 depicts a permeate analysis of PHB particles purified by TFF with 0.2 Deoxycholate, as described in Example 10 herein. As indicated, symbols represent A260 (Δ) and A280 nm (▪) absorbance measurements of permeate fractions collected over 12 diafiltration volumes, with the measured pH (∘) also shown. The particles were diafiltered against 8 volumes of 10 mM Tris 10 mM EDTA 0.2% Deoxycholate pH 11 followed by 4 volumes of PBS, pH 7.4.

FIG. 19 shows an assay of the IgG binding capacity of polymer particles of the present invention comprising the Z-domain after TFF purification, as described in Example 10 herein. After diafiltration, in a range of 0.2% Deoxycholate containing solutions (Table 2), 50 mg aliquots of beads were incubated with 5 mg of human IgG for 30 min at room temperature in PBS, pH 7.4. After incubation the polymer particles were centrifuged to remove the unbound fraction and then eluted with Glycine buffer pH 2.7 to elute IgG from the polymer particles of the present invention comprising the Z-domain. The polymer particles were re-centrifuged and the recovered IgG in the eluate supernatant was determined using the Bradford protein assay.

FIG. 20 depicts permeate analysis of polymer particles of the present invention comprising the GB1-domain using an open channel TFF system, as described herein in Example 11. Lubrol-extracted PHB polymer particles were loaded onto a Millipore Prostak—4 stak system. The beads were suspended to create a 1.3 liter retentate, and 1 liter permeate fractions were collected and analysed.

FIG. 21 shows an assay of the IgG binding capacity of polymer particles of the present invention comprising the GB1-domain after TFF Purification, as described herein in Example 11. After purification from either a glycerol gradient or the TFF-lubrol based process, 50 mg aliquots of polymer particles were incubated with 5 mg of human IgG for 30 min at room temperature in PBS, pH 7.4. After incubation the polymer particles were centrifuged to remove the unbound fraction and then eluted with Glycine buffer pH 2.7 to elute IgG from the polymer particles. The polymer particles were re-centrifuged and the recovered IgG in the eluate supernatant was determined using the Bradford protein assay.

FIG. 22 are graphs depicting the effect of various extraction agents on host cell protein/nucleic acid removal and residual binding activity from polymer particles of the present invention comprising the Z-domain, in cell extracts. FIG. 22A shows the A260 nm and A280 nm absorbance results in batch wash supernatants diluted in PBS, 20% (where required). FIG. 22B shows IgG binding activity, where crude polymer particle pellets were washed 1× in PBS, centrifuged at 15,000×g for 20 minutes and the drained pellet was weighed and the weight-normalized IgG binding activity was determined.

FIG. 23 shows a flexible scale scheme for purifying PHB polymer particles from bacterial biomass.

FIG. 24 shows the removal of host cell biomass from PHB polymer particles by SDS detergent extraction as described herein in Example 14. Two separate replicate sub-batches of biomass (1.1-1.2 kg) were suspended to 2.7 litres in 0.08% SDS, 25 mM Tris 10 mM EDTA, pH 11 and microfluidized. Sequential chemical washes in lysis buffer, 10 mM Thioglycerol and 0.1 M NaOH were performed at 2.7 L, 1 L and 1 L volumes respectively. The wet weight of the crude polymer particles was determined at each process step.

FIG. 25 depicts a permeate analysis of polymer particles of the present invention comprising the Z-domain purified with a SDS based TFF purification process as described herein in FIG. 14. SDS extracted PHB beads were loaded onto a Millipore Prostak system with 3. Two stak modules in the system (0.41 m²). The beads were suspended to create a 2 liter retentate and two liter permeate fractions, then collected and analysed for 260, 280, 600 nm absorbance and pH.

FIG. 26 depicts SDS-PAGE analysis of PHB beads purified with a SDS-based TFF process as described in Example 14. Aliquots (25 μl) with 5 ul sample buffer and 20 μl of each sample was loaded on an 8-15% gradient gel. Samples as marked are 1. MW marker, 2. Crude cell lysate, 3. Polymer particles post lysis, 4. Polymer particles post lysis buffer (SDS) wash, 5. Polymer particles post thioglycerol wash, 6. Polymer particles post NaOH wash (pre TFF) 7. Polymer particles post TFF purification.

### DETAILED DESCRIPTION OF THE INVENTION

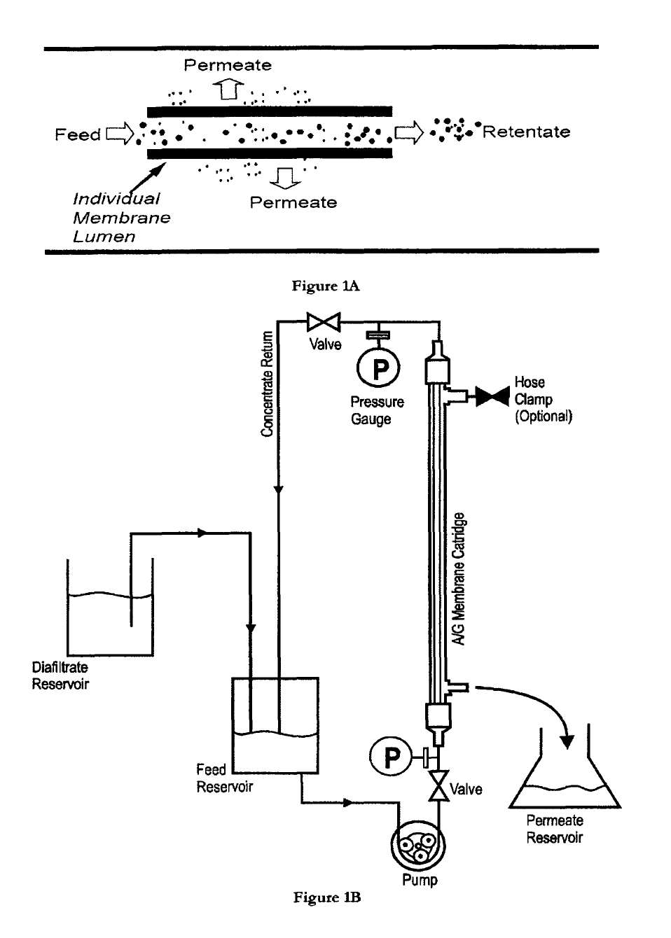

The present invention relates to methods and compositions for use in tangential-flow filtration techniques. Tangential flow filtration is a separation technology in which the feedstock is run tangentially to the membrane (as opposed to substantially perpendicular to the membrane in dead-end filtration). This tangential-flow creates a pressure differential across the membrane. As a result, some particles pass through the membrane, while other particles continue to flow across the membrane (see FIG. 1A) which in certain cases serves to clean the membrane. When compared to dead-end filtration, a tangential-flow will generally slow the build-up of particles into a filter cake. Other benefits realised by tangential-flow filtration systems will be well known to those skilled in the art, and include high liquid volume capacity, high target substance-binding capacity, ease of regeneration of the polymer particles, chromatography resins, membranes, and the like.

### DEFINITIONS

As used herein, the term “amorphous polymer” is to be understood as those polymers which are solids at room temperature in spite of an irregular arrangement of the molecule chains. These polymers are essentially non-crystalline, and their degree of crystallinity is typically below 20%, preferably below 15%, below 10%, below 5%, preferably below 2%, or is 0%. Those amorphous polymers are particularly suitable whose glass transition temperature T_{G} is in the range from 0° to 60° C., preferably 0° to 50° C., preferably 0° to 40° C., preferably 0° to 35° C., and in particular 0° to 30° C.

As used herein, the term “biopolymer” is to be understood as those polymers which are able to be synthesised by a biological system or entity, such as but not limited to an organism, a cell, or a protein. Accordingly, the terms “biopolyester” and “biopolythioester” is to be understood as those polyesters, and polythioesters, respectively, which are able to be synthesised by a biological system or entity. Examples include polyesters and polyhydroxycarboxylates produced by various bacteria and archea, typically as a means to store carbon or energy, such as but not limited to polythioesters and polyhydroxyalkanoates.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “contaminant” refers to a substance or substances in the source material that differ from the target substance, and are desirably excluded from the final target substance preparation. Typical contaminants of biological source materials include nucleic acids, proteins, peptides, endotoxins, viruses, etc. Contaminants that can be removed by the practice of the inventive method have one or more properties that differ from those of the desired product, e.g., molecular weight, charge, specific affinity for various ligands, and so on.

The term “tangential-flow filter” and grammatical equivalents refers herein to a type of filter module or filter cassette that comprises a porous, permeable or semipermeable filter element across a surface of which the source medium to be filtered is flowed in a tangential-flow fashion, for example for permeation through the filter element of selected component(s) or contaminants of the source medium.

The term “coupling reagent” as used herein refers to an inorganic or organic compound that is suitable for binding at least one substance or a further coupling reagent that is suitable for binding a coupling reagent on one side and at least one substance on the other side. Examples of suitable coupling reagents, as well as exemplary methods for their use including methods suitable for the chemical modification of particles or fusion proteins of the present invention, are presented in PCT/DE2003/002799, published as WO 2004/020623 (Bernd Rehm), herein incorporated by reference in its entirety.

The term “expression construct” refers to a genetic construct that includes elements that permit transcribing the insert polynucleotide molecule, and, optionally, translating the transcript into a polypeptide. An expression construct typically comprises in a 5′ to 3′ direction:

(1) a promoter, functional in the host cell into which the construct will be introduced,
(2) the polynucleotide to be expressed, and
(3) a terminator functional in the host cell into which the construct will be introduced.

Expression constructs of the invention are inserted into a replicable vector for cloning or for expression, or are incorporated into the host genome.

Examples of expression constructs amenable for adaptation for use in the present invention are provided in PCT/DE2003/002799 published as WO 2004/020623 (Bernd Rehm) and PCT/NZ2006/000251 published as WO 2007/037706 (Bernd Rehm) which are each herein incorporated by reference in their entirety.

The terms “form a polymer particle” and “formation of polymer particles”, as used herein in relation to particle-forming proteins refer to the activity of a particle-forming protein as discussed herein.

A “fragment” of a polypeptide is a subsequence of the polypeptide that performs a function that is required for the enzymatic or binding activity and/or provides three dimensional structure of the polypeptide.

The term “fusion polypeptide”, as used herein, refers to a polypeptide comprising two or amino acid sequences, for example two or more polypeptide domains, fused through respective amino and carboxyl residues by a peptide linkage to form a single continuous polypeptide. It should be understood that the two or more amino acid sequences can either be directly fused or indirectly fused through their respective amino and carboxyl terimini through a linker or spacer or an additional polypeptide.

In one embodiment, one of the amino acid sequences comprising the fusion polypeptide comprises a particle-forming protein. In one embodiment, one of the amino acid sequences comprising the fusion polypeptide comprises a polymer synthase.

In one embodiment, one of the amino acid sequences comprising the fusion polypeptide comprises a fusion partner.

The term “fusion partner” as used herein refers to a polypeptide such as a protein, a protein fragment, a binding domain, a target-binding domain, a binding protein, a binding protein fragment, an antibody, an antibody fragment, an antibody heavy chain, an antibody light chain, a single chain antibody, a single-domain antibody (a VHH for example), a Fab antibody fragment, an Fc antibody fragment, an Fv antibody fragment, a F(ab′)2 antibody fragment, a Fab′ antibody fragment, a single-chain Fv (scFv) antibody fragment, an antibody binding domain (a ZZ domain for example), an antigen, an antigenic determinant, an epitope, a hapten, an immunogen, an immunogen fragment, biotin, a biotin derivative, an avidin, a streptavidin, a substrate, an enzyme, an abzyme, a co-factor, a receptor, a receptor fragment, a receptor subunit, a receptor subunit fragment, a ligand, an inhibitor, a hormone, a lectin, a polyhistidine, a coupling domain, a DNA binding domain, a FLAG epitope, a cysteine residue, a library peptide, a reporter peptide, an affinity purification peptide, or any combination of any two or more thereof.

It should be understood that two or more polypeptides listed above can form the fusion partner.

In one embodiment the amino acid sequences of the fusion polypeptide are indirectly fused through a linker or spacer, the amino acid sequences of said fusion polypeptide arranged in the order of polymer synthase-linker-fusion partner, or fusion partner-linker-polymer synthase. In other embodiments the amino acid sequences of the fusion polypeptide are indirectly fused through or comprise an additional polypeptide arranged in the order of polymer synthase-additional polypeptide-fusion partner, or polymer synthase-linker-fusion partner-additional polypeptide. Again, N-terminal extensions of the polymer synthase are expressly contemplated herein.

In one exemplary embodiment the amino acid sequences of the fusion polypeptide are indirectly fused through a linker or spacer, the amino acid sequences of said fusion polypeptide arranged in the order of polymer synthase-linker-antibody binding polypeptide or antibody binding polypeptide-linker-polymer synthase, or polymer synthase-linker-enzyme or enzyme-linker-polymer synthase, for example. In other exemplary embodiments the amino acid sequences of the fusion polypeptide are indirectly fused through or comprise an additional polypeptide arranged in the order of polymer synthase-additional polypeptide-antibody binding polypeptide or polymer synthase-additional polypeptide-enzyme, or polymer synthase-linker-antibody binding polypeptide-additional polypeptide or polymer synthase-linker-enzyme-additional polypeptide. Again, N-terminal extensions of the polymer synthase are expressly contemplated herein.

A fusion polypeptide according to the invention may also comprise one or more polypeptide sequences inserted within the sequence of another polypeptide. For example, a polypeptide sequence such as a protease recognition sequence is inserted into a variable region of a protein comprising a particle binding domain

Conveniently, a fusion polypeptide of the invention is encoded by a single nucleic acid sequence, wherein the nucleic acid sequence comprises at least two subsequences each encoding a polypeptide or a polypeptide domain. In certain embodiments, the at least two subsequences will be present “in frame” so as comprise a single open reading frame and thus will encode a fusion polypeptide as contemplated herein. In other embodiments, the at least two subsequences are present “out of frame”, and are separated by a ribosomal frame-shifting site or other sequence that promotes a shift in reading frame such that, on translation, a fusion polypeptide is formed. In certain embodiments, the at least two subsequences are contiguous. In other embodiments, such as those discussed above where the at least two polypeptides or polypeptide domains are indirectly fused through an additional polypeptide, the at least two subsequences are not contiguous.

Reference to a “binding domain” or a “domain capable of binding” is intended to mean one half of a complementary binding pair and may include binding pairs from the list above. For example, antibody-antigen, antibody-antibody binding domain, biotin-streptavidin, receptor-ligand, enzyme-inhibitor pairs. A target-binding domain will bind a target molecule in a sample, and are an antibody or antibody fragment, for example. A polypeptide-binding domain will bind a polypeptide, and are an antibody or antibody fragment, or a binding domain from a receptor or signalling protein, for example.

Examples of substances that are bound by a binding domain include a protein, a protein fragment, a peptide, a polypeptide, a polypeptide fragment, an antibody, an antibody fragment, an antibody binding domain, an antigen, an antigen fragment, an antigenic determinant, an epitope, a hapten, an immunogen, an immunogen fragment, a pharmaceutically active agent, a biologically active agent, an adjuvant or any combination of any two or more thereof. Such substances are “target components” in a sample that is analysed according to a method of the invention.

Accordingly, a “domain capable of binding a target substance” and grammatical equivalents will be understood to refer to one component in a complementary binding pair, wherein the other component is the target substance.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. In various embodiments, the insert polynucleotide molecule is derived from the host cell, or is derived from a different cell or organism and/or is a recombinant polynucleotide. In one embodiment, once inside the host cell the genetic construct becomes integrated in the host genome, such as the host chromosomal DNA. In one example the genetic construct is linked to a vector.

The term “host cell” refers to a bacterial cell, a fungi cell, yeast cell, a plant cell, an insect cell or an animal cell such as a mammalian host cell that is either 1) a natural PHA particle producing host cell, or 2) a host cell carrying an expression construct comprising nucleic acid sequences encoding at least a thiolase and a reductase and optionally a phasin. Which genes are required to augment what the host cell lacks for polymer particle formation will be dependent on the genetic makeup of the host cell and which substrates are provided in the culture medium.

The term “linker or spacer” as used herein relates to an amino acid or nucleotide sequence that indirectly fuses two or more polypeptides or two or more nucleic acid sequences encoding two or more polypeptides. In some embodiments the linker or spacer is about 1, 5, 10, 15, 20, 25, 30, 35, 40, 45, 50, 55, 60, 65, 70, 75, 80, 85, 90, 95 or about 100 amino acids or nucleotides in length. In other embodiments the linker or spacer is about 100, 125, 150, 175, 200, 225, 250, 275, 300, 325, 350, 375, 400, 450, 500, 550, 600, 650, 700, 750, 800, 850, 900, 950 or about 1000 amino acids or nucleotides in length. In still other embodiments the linker or spacer is from about 1 to about 1000 amino acids or nucleotides in length, from about 10 to about 1000, from about 50 to about 1000, from about 100 to about 1000, from about 200 to about 1000, from about 300 to about 1000, from about 400 to about 1000, from about 500 to about 1000, from about 600 to about 1000, from about 700 to about 1000, from about 800 to about 1000, or from about 900 to about 1000 amino acids or nucleotides in length.

In one embodiment the linker or spacer may comprise a restriction enzyme recognition site. In another embodiment the linker or spacer may comprise a protease cleavage recognition sequence such as enterokinase, thrombin or Factor Xa recognition sequence, or a self-splicing element such as an intein. In another embodiment the linker or spacer facilitates independent folding of the fusion polypeptides.

The term “mixed population”, as used herein, refers to two or more populations of entities, each population of entities within the mixed population differing in some respect from another population of entities within the mixed population. For example, when used in reference to a mixed population of expression constructs, this refers to two or more populations of expression constructs where each population of expression construct differs in respect of the fusion polypeptide encoded by the members of that population, or in respect of some other aspect of the construct, such as for example the identity of the promoter present in the construct. Alternatively, when used in reference to a mixed population of fusion polypeptides, this refers to two or more populations of fusion polypeptides where each population of fusion polypeptides differs in respect of the polypepetides, such as polymer synthase, the fusion partner such as an antibody binding domain or an enzyme, the members that population contains. For example, in the context of use in the preparation of a purified antibody, a mixed population of fusion polypeptides refers to two or more populations of fusion polypeptides where each population of fusion polypeptides differs in respect of the polypepetides, such as polymer synthase, the antibody binding domain, the members that population contains. Similarly, in the context of use in the preparation of a target substance a mixed population of fusion polypeptides refers to two or more populations of fusion polypeptides where each population of fusion polypeptides differs in respect of the polypepetides, such as polymer synthase, the enzyme, the precursor binding domain, or the enzyme-substrate binding domain the members that population contains. Still further, when used in reference to a mixed population of polymer particles, this refers to two or more populations of polymer particles where each population of polymer particles differs in respect of the fusion polypeptide or fusion polypeptides the members of that population carry. Mixed populations of polymer particles comprising two or more subpopulation of polymer particles, where each subpopulation may comprise one or more of the fusion polypeptides described herein (such as those above) are specifically contemplated.

The term “nucleic acid” as used herein refers to a single- or double-stranded polymer of deoxyribonucleotide, ribonucleotide bases or known analogues of natural nucleotides, or mixtures thereof. The term includes reference to a specified sequence as well as to a sequence complimentary thereto, unless otherwise indicated. The terms “nucleic acid” and “polynucleotide” are used herein interchangeably.

“Operably-linked” means that the sequenced to be expressed is placed under the control of regulatory elements that include promoters, tissue-specific regulatory elements, temporal regulatory elements, enhancers, repressors and terminators.

The term “over-expression” generally refers to the production of a gene product in a host cell that exceeds levels of production in normal or non-transformed host cells. The term “overexpression” when used in relation to levels of messenger RNA preferably indicates a level of expression at least about 3-fold higher than that typically observed in a host cell in a control or non-transformed cell. More preferably the level of expression is at least about 5-fold higher, about 10-fold higher, about 15-fold higher, about 20-fold higher, about 25-fold higher, about 30-fold higher, about 35-fold higher, about 40-fold higher, about 45-fold higher, about 50-fold higher, about 55-fold higher, about 60-fold higher, about 65-fold higher, about 70-fold higher, about 75-fold higher, about 80-fold higher, about 85-fold higher, about 90-fold higher, about 95-fold higher, or about 100-fold higher or above, than typically observed in a control host cell or non-transformed cell.

Levels of mRNA are measured using any of a number of techniques known to those skilled in the art including, but not limited to, Northern blot analysis and RT-PCR, including quantitative RT-PCR.

The term “particle-binding protein”, as used herein refers to proteins and protein domains capable of binding to the particle. Such binding may be mediated directly through interaction with the polymer, or via interaction with a moiety bound to the polymer, such as via a polymer synthase covalently bound to the polymer. Particle-binding proteins suitable for use herein include one or more particle binding domains from proteins capable of binding to the polymer particle core, such as the C-terminal fragment of PHA synthase protein or the particle binding domain of polymer depolymerise.

The term “particle-forming protein”, as used herein, refers to proteins involved in the formation of the particle. It may, for example, be selected from the group of proteins which comprises a polymer depolymerase, a polymer regulator, a polymer synthase and a particle size-determining protein. Preferably the particle-forming protein is selected from the group comprising a thiolase, a reductase, a polymer synthase and a phasin. A particle-forming protein such as a synthase may catalyse the formation of a polymer particle by polymerising a substrate or a derivative of a substrate to form a polymer particle. Alternatively, a particle-forming protein such as a thiolase, a reductase or a phasin may facilitate the formation of a polymer particle by facilitating polymerisation. For example, a thiolase or reductase may catalyse production of suitable substrates for a polymerase. A phasin may control the size of the polymer particle formed. Preferably the particle-forming protein comprises a particle binding domain and a particle forming domain.

As used herein, the term “particle-forming reaction mixture” refers to at least a polymer synthase substrate if the host cell or expression construct comprises a synthase catalytic domain or a polymer synthase and its substrate if the host cell or expression construct comprises another particle-forming protein or a particle binding domain that is not a polymer synthase catalytic domain

A “particle size-determining protein” refers to a protein that controls the size of the polymer particles. It may for example be derived from the family of phasin-like proteins, preferably selected from the those from the genera Ralstonia, Alcaligenes and Pseudomonas, more preferably the phasin gene phaP from Ralstonia eutropha and the phasin gene phaF from Pseudomonas oleovorans. Phasins are amphiphilic proteins with a molecular weight of 14 to 28 kDa which bind tightly to the hydrophobic surface of the polymer particles. It may also comprise other host cell proteins that bind particles and influence particle size.

A polymer synthase comprises at least the synthase catalytic domain at the C-terminus of the synthase protein that mediates polymerisation of the polymer and attachment of the synthase protein to the particle core. Polymer synthases for use in the present invention are described in detail in Rehm, 2003, which is herein incorporated by reference in its entirety. For example, the polymer synthase is a PHA synthase from the class 1 genera Acinetobacter, Vibrio, Aeromonas, Chromobacterium, Pseudomonas, Zoogloea, Alcaligenes, Delftia, Burkholderia, Ralstonia, Rhodococcus, Gordonia, Rhodobacter, Paracoccus, Rickettsia, Caulobacter, Methylobacterium, Azorhizobium, Agrobacterium, Rhizobium, Sinorhizobium, Rickettsia, Crenarchaeota, Synechogstis, Ectothiorhodospira, Thiocapsa, Thyogstis and Allochromatium, the class 2 genera Burkholderia and Pseudomonas, or the class 4 genera Bacillus, more preferably from the group comprising class 1 Acinetobacter sp. RA3849, Vibrio cholerae, Vibrio parahaemolyticus, Aeromonas punctata FA440, Aeromonas hydrophila, Chromobacterium violaceum, Pseudomonas sp. 61-3, Zoogloea ramigera, Alcaligenes latus, Alcaligenes sp. SH-69, Delftia acidovorans, Burkholderia sp. DSMZ9242, Ralstonia eutrophia H16, Burkholderia cepacia, Rhodococcus rubber PP2, Gordonia rubripertinctus, Rickettsia prowazekii, Synechocystis sp. PCC6803, Ectothiorhodospira shaposhnikovii N1, Thiocapsa pfennigii 9111, Allochromatium vinosum D, Thyogstis violacea 2311, Rhodobacter sphaeroides, Paracoccus denitrificans, Rhodobacter capsulatus, Caulobacter crescentus, Methylobacterium extorquens, Azorhizobium caulinodans, Agrobacterium tumefaciens, Sinorhizobium meliloti 41, Rhodospirillum rubrum HA, and Rhodospirillum rubrum ATCC25903, class 2 Burkholderia caryophylli, Pseudomonas chloraphis, Pseudomonas sp. 61-3, Pseudomonas putida U, Pseudomonas oleovorans, Pseudomonas aeruginosa, Pseudomonas resinovorans, Pseudomonas stutzeri, Pseudomonas mendocina, Pseudomonas pseudolcaligenes, Pseudomonas putida BM01, Pseudomonas nitroreducins, Pseudomonas chloraphis, and class 4 Bacillus megaterium and Bacillus sp. INT005.

Other polymer synthases amenable to use in the present invention include polymer synthases, each identified by it accession number, from the following organisms: C. necator (AY836680), P. aeruginosa (AE004091), A. vinosum (AB205104), B. megaterium (AF109909), H. marismortui (YP137339), P. aureofaciens (AB049413), P. putida (AF150670), R. eutropha (A34341), T. pfennigii (X93599), A. punctata (32472), Pseudomonas 61-3 (AB014757 and AB014758), R. sphaeroides (AAA72004, C. violaceum (AAC69615), A. borkumensis SK2 (CAL17662), A. borkumensis SK2 (CAL16866), R. sphaeroides KD131 (ACM01571 AND YP002526072), R. opacus B4 (BAH51880 and YP002780825), B. multivorans ATCC 17616 (YP001946215 and BAG43679), A. borkumensis SK2(YP693934 and YP693138), R. rubrum (AAD53179), gamma proteobacterium HTCC5015 (ZP05061661 and EDY86606), Azoarcus sp. BH72 (YP932525), C. violaceum ATCC 12472 (NP902459), Limnobacter sp. MED105 (ZP01915838 and EDM82867), M. algicola DG893 (ZP01895922 and EDM46004), R. sphaeroides (CAA65833), C. violaceum ATCC 12472 (AAQ60457), A. latus (AAD10274, AAD01209 and AAC83658), S. maltophilia K279a (CAQ46418 and YP001972712), R. solanacearum IPO1609 (CAQ59975 and YP002258080), B. multivorans ATCC 17616 (YP001941448 and BAG47458), Pseudomonas sp. gl13 (ACJ02400), Pseudomonas sp. gl06 (ACJ02399), Pseudomonas sp. gl01 (ACJ02398), R. sp. gl32 (ACJ02397), R. leguminosarum bv. viciae 3841 (CAK10329 and YP770390), Aoarcus sp. BH72 (CAL93638), Pseudomonas sp. LDC-5 (AAV36510), L. nitroferrum 2002 (ZP03698179), Thauera sp. MZ1T (YP002890098 and ACR01721), M. radiotolerans JCM 2831 (YP001755078 and ACB24395), Methylobacterium sp. 4-46 (YP001767769 and ACA15335), L. nitroferrum 2002 (EEG08921), P. denitrificans (BAA77257), M. gryphiswaldense (ABG23018), Pseudomonas sp. USM4-55 (ABX64435 and ABX64434), A. hydrophila (AAT77261 and AAT77258), Bacillus sp. INT005 (BAC45232 and BAC45230), P. putida (AAM63409 and AAM63407), G. rubripertinctus (AAB94058), B. megaterium (AAD05260), D. acidovorans (BAA33155), P. seriniphilus (ACM68662), Pseudomonas sp. 14-3 (CAK18904), Pseudomonas sp. LDC-5 (AAX18690), Pseudomonas sp. PC17 (ABV25706), Pseudomonas sp. 3Y2 (AAV35431, AAV35429 and AAV35426), P. mendocina (AAM10546 and AAM10544), P. nitroreducens (AAK19608), P. pseudoalcaligenes (AAK19605), P. resinovorans (AAD26367 and AAD26365), Pseudomonas sp. USM7-7 (ACM90523 and ACM90522), P. fluorescens (AAP58480) and other uncultured bacterium (BAE02881, BAE02880, BAE02879, BAE02878, BAE02877, BAE02876, BAE02875, BAE02874, BAE02873, BAE02872, BAE02871, BAE02870, BAE02869, BAE02868, BAE02867, BAE0286, BAE02865, BAE02864, BAE02863, BAE02862, BAE02861, BAE02860, BAE02859, BAE02858, BAE02857, BAE07146, BAE07145, BAE07144, BAE07143, BAE07142, BAE07141, BAE07140, BAE07139, BAE07138, BAE07137, BAE07136, BAE07135, BAE07134, BAE07133, BAE07132, BAE07131, BAE07130, BAE07129, BAE07128, BAE07127, BAE07126, BAE07125, BAE07124, BAE07123, BAE07122, BAE07121, BAE07120, BAE07119, BAE07118, BAE07117, BAE07116, BAE07115, BAE07114, BAE07113, BAE07112, BAE07111, BAE07110, BAE07109, BAE07108, BAE07107, BAE07106, BAE07105, BAE07104, BAE07103, BAE07102, BAE07101, BAE07100, BAE07099, BAE07098, BAE07097, BAE07096, BAE07095, BAE07094, BAE07093, BAE07092, BAE07091, BAE07090, BAE07089, BAE07088, BAE07053, BAE07052, BAE07051, BAE07050, BAE07049, BAE07048, BAE07047, BAE07046, BAE07045, BAE07044, BAE07043, BAE07042, BAE07041, BAE07040, BAE07039, BAE07038, BAE07037, BAE07036, BAE07035, BAE07034, BAE07033, BAE07032, BAE07031, BAE07030, BAE07029, BAE07028, BAE07027, BAE07026, BAE07025, BAE07024, BAE07023, BAE07022, BAE07021, BAE07020, BAE07019, BAE07018, BAE07017, BAE07016, BAE07015, BAE07014 BAE07013, BAE07012, BAE07011, BAE07010 BAE07009, BAE07008, BAE07007 BAE07006 BAE07005, BAE07004, BAE07003, BAE07002 BAE07001, BAE07000, BAE06999, BAE06998, BAE06997, BAE06996, BAE06995, BAE06994, BAE06993, BAE06992, BAE06991, BAE06990, BAE06989, BAE06988, BAE06987, BAE06986, BAE06985, BAE06984, BAE06983, BAE06982, BAE06981, BAE06980, BAE06979, BAE06978, BAE06977, BAE06976, BAE06975, BAE06974, BAE06973, BAE06972, BAE06971, BAE06970, BAE06969, BAE06968, BAE06967, BAE06966, BAE06965, BAE06964, BAE06963, BAE06962, BAE06961, BAE06960, BAE06959, BAE06958, BAE06957, BAE06956, BAE06955, BAE06954, BAE06953, BAE06952, BAE06951, BAE06950, BAE06949, BAE06948, BAE06947, BAE06946, BAE06945, BAE06944, BAE06943, BAE06942, BAE06941, BAE06940, BAE06939, BAE06938, BAE06937, BAE06936, BAE06935, BAE06934, BAE06933, BAE06932, BAE06931, BAE06930, BAE06929, BAE06928, BAE06927, BAE06926, BAE06925, BAE06924, BAE06923, BAE06922, BAE06921, BAE06920, BAE06919, BAE06918, BAE06917, BAE06916, BAE06915, BAE06914, BAE06913, BAE06912, BAE06911, BAE06910, BAE06909, BAE06908, BAE06907, BAE06906, BAE06905, BAE06904, BAE06903, BAE06902, BAE06901, BAE06900, BAE06899, BAE06898, BAE06897, BAE06896, BAE06895, BAE06894, BAE06893, BAE06892, BAE06891, BAE06890, BAE06889, BAE06888, BAE06887, BAE06886, BAE06885, BAE06884, BAE06883, BAE06882, BAE06881, BAE06880, BAE06879, BAE06878, BAE06877, BAE06876, BAE06875, BAE06874, BAE06873, BAE06872, BAE06871, BAE06870, BAE06869, BAE06868, BAE06867, BAE06866, BAE06865, BAE06864, BAE06863, BAE06862, BAE06861, BAE06860, BAE06859, BAE06858, BAE06857, BAE06856, BAE06855, BAE06854, BAE06853 and BAE06852).

The N-terminal fragment of PHA synthase protein (about amino acids 1 to 200, or 1 to 150, or 1 to 100) is highly variable and in some examples is deleted or replaced by an enzyme, an antibody binding domain, or another fusion partner without inactivating the synthase or preventing covalent attachment of the synthase via the polymer particle binding domain (i.e. the C-terminal fragment) to the polymer core. The polymer particle binding domain of the synthase comprises at least the catalytic domain of the synthase protein that mediates polymerisation of the polymer core and formation of the polymer particles.

In some embodiments the C-terminal fragment of PHA synthase protein is modified, partially deleted or partially replaced by an enzyme, an antibody binding domain, or another fusion partner without inactivating the synthase or preventing covalent attachment of the synthase to the polymer particle.

In certain cases, the enzyme, the antibody binding domain, or another fusion partner are fused to the N-terminus and/or to the C-terminus of PHA synthase protein without inactivating the synthase or preventing covalent attachment of the synthase to the polymer particle. Similarly, in other cases the enzyme, the antibody binding domain, or another fusion partner, are inserted within the PHA synthase protein, or indeed within the particle-forming protein. Examples of PhaC fusions are known in the art and presented herein.

In one specific example, the N-terminal fragment of PHA synthase protein (about amino acids 1 to 200, or 1 to 150, or 1 to 100) is deleted or replaced by an antibody binding domain such as the Z domain of protein A or a tandem repeat of same without inactivating the synthase or preventing covalent attachment of the synthase to the polymer particle.

A “polymer depolymerase” as used herein refers to a protein which is capable of hydrolysing existing polymer, such as that found in a polymer particle, into water soluble monomers and oligomers. Examples of polymer depolymerases occur in a wide variety of PHA-degrading bacteria and fungi, and include the PhaZ1-PhaZ7 extracellular depolymerases from Paucimonas lemoignei, the PhaZ depolymerases from Acidovorax sp., A. faecalis (strains AE122 and T1), Delia (Comamonas) acidovorans strain YM1069, Comamonas testosteroni, Comamonas sp., Leptothrix sp. strain HS, Pseudomonas sp. strain GM101 (acession no. AF293347), P. fluorescens strain GK13, P. stutzeri, R. pickettii (strains A1 and K1, acession no. JO4223, D25315), S. exfoliatus K10 and Streptomyces hygroscopicus (see Jendrossek D., and Handrick, R., Microbial Degredation of Polyhydroxyalkanoates, Annual Review of Microbiology, 2002, 56:403-32).

The term “polypeptide”, as used herein, encompasses amino acid chains of any length but preferably at least 5 amino acids, including full-length proteins, in which amino acid residues are linked by covalent peptide bonds. Polypeptides of the present invention are purified natural products, or are produced partially or wholly using recombinant or synthetic techniques. The term may refer to a polypeptide, an aggregate of a polypeptide such as a dimer or other multimer, a fusion polypeptide, a polypeptide variant, or derivative thereof.

The term “promoter” refers to non transcribed cis-regulatory elements upstream of the coding region that regulate gene transcription. Promoters comprise cis-initiator elements which specify the transcription initiation site and conserved boxes such as the TATA box, and motifs that are bound by transcription factors.

The term “terminator” refers to sequences that terminate transcription, which are found in the 3′ untranslated ends of genes downstream of the translated sequence. Terminators are important determinants of mRNA stability and in some cases have been found to have spatial regulatory functions.

The term “substance” when referred to in relation to being bound to or absorbed into or incorporated within a polymer particle is intended to mean a substance that is bound by a fusion partner or a substance that is able to be absorbed into or incorporated within a polymer particle.

The term “variant” as used herein refers to polynucleotide or polypeptide sequences different from the specifically identified sequences, wherein one or more nucleotides or amino acid residues is deleted, substituted, or added. Variants are naturally-occurring allelic variants, or non-naturally occurring variants. Variants are from the same or from other species and may encompass homologues, paralogues and orthologues. In certain embodiments, variants of the polynucleotides and polypeptides possess biological activities that are the same or similar to those of the wild type polynucleotides or polypeptides. The term “variant” with reference to polynucleotides and polypeptides encompasses all forms of polynucleotides and polypeptides as defined herein.

### Polynucleotide and Polypeptide Variants

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments. A number of nucleic acid analogues are well known in the art and are also contemplated.

A “fragment” of a polynucleotide sequence provided herein is a subsequence of contiguous nucleotides that is preferably at least 15 nucleotides in length. The fragments of the invention preferably comprises at least 20 nucleotides, more preferably at least 30 nucleotides, more preferably at least 40 nucleotides, more preferably at least 50 nucleotides and most preferably at least 60 contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods.

The term “fragment” in relation to promoter polynucleotide sequences is intended to include sequences comprising cis-elements and regions of the promoter polynucleotide sequence capable of regulating expression of a polynucleotide sequence to which the fragment is operably linked

Preferably fragments of promoter polynucleotide sequences of the invention comprise at least 20, more preferably at least 30, more preferably at least 40, more preferably at least 50, more preferably at least 100, more preferably at least 200, more preferably at least 300, more preferably at least 400, more preferably at least 500, more preferably at least 600, more preferably at least 700, more preferably at least 800, more preferably at least 900 and most preferably at least 1000 contiguous nucleotides of a promoter polynucleotide of the invention.

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

The term “probe” refers to a short polynucleotide that is used to detect a polynucleotide sequence that is complementary to the probe, in a hybridization-based assay. The probe may consist of a “fragment” of a polynucleotide as defined herein. Preferably such a probe is at least 5, more preferably at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

The term “variant” as used herein refers to polynucleotide or polypeptide sequences different from the specifically identified sequences, wherein one or more nucleotides or amino acid residues is deleted, substituted, or added. Variants are naturally-occurring allelic variants, or non-naturally occurring variants. Variants are from the same or from other species and may encompass homologues, paralogues and orthologues. In certain embodiments, variants of the polynucleotides and polypeptides possess biological activities that are the same or similar to those of the wild type polynucleotides or polypeptides. The term “variant” with reference to polynucleotides and polypeptides encompasses all forms of polynucleotides and polypeptides as defined herein.

### Polynucleotide Variants

Variant polynucleotide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a specified polynucleotide sequence. Identity is found over a comparison window of at least 20 nucleotide positions, preferably at least 50 nucleotide positions, at least 100 nucleotide positions, or over the entire length of the specified polynucleotide sequence.

Polynucleotide sequence identity can be determined in the following manner. The subject polynucleotide sequence is compared to a candidate polynucleotide sequence using BLASTN (from the BLAST suite of programs, version 2.2.10 [October 2004]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences can be examined using the following unix command line parameters:

bl2seq-i nucleotideseq1-j nucleotideseq2-F F-p blastn

The parameter-F F turns off filtering of low complexity sections. The parameter-p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program can be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides determined using the publicly available bl2seq program from the BLAST suite of programs (version 2.2.10 [October 2004]) from NCBI (ftp://ftp.ncbi.nih gov/blast/).

The similarity of polynucleotide sequences can be examined using the following unix command line parameters:

bl2seq-i nucleotideseq1-j nucleotideseq2-F F-p tblastx

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10¹¹⁰, less than 1×10¹²⁰ or less than 1×10⁻¹²³ when compared with any one of the specifically identified sequences.

Alternatively, variant polynucleotides of the present invention hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C-log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), in some examples other codons for the same amino acid are changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence can be determined using the publicly available bl2seq program from the BLAST suite of programs (version 2.2.10 [October 2004]) from NCBI (ftp://ftp.ncbi.nih.gov/blast/) via the tblastx algorithm as previously described.

### Polypeptide Variants

The term “variant” with reference to polypeptides encompasses naturally occurring, recombinantly and synthetically produced polypeptides. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least %, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequences of the present invention. Identity is found over a comparison window of at least 20 amino acid positions, preferably at least 50 amino acid positions, at least 100 amino acid positions, or over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Polypeptide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides can be determined using the publicly available bl2seq program from the BLAST suite of programs (version 2.2.10 [October 2004]) from NCBI (ftp://ftp.ncbi.nih gov/blast/). The similarity of polypeptide sequences can be examined using the following unix command line parameters:

bl2seq-i peptideseq1-j peptideseq2-F F-p blastp

Variant polypeptide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10¹⁰⁰, less than 1×10⁻¹¹⁰, less than 1×10¹²⁰ or less than 1×10⁻¹²³ when compared with any one of the specifically identified sequences.

The parameter-F F turns off filtering of low complexity sections. The parameter-p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

A polypeptide variant of the present invention also encompasses that which is produced from the nucleic acid encoding a polypeptide, but differs from the wild type polypeptide in that it is processed differently such that it has an altered amino acid sequence. For example, a variant is produced by an alternative splicing pattern of the primary RNA transcript to that which produces a wild type polypeptide.

The term “vector” refers to a polynucleotide molecule, usually double stranded DNA, which is used to transport the genetic construct into a host cell. In certain examples the vector is capable of replication in at least one additional host system, such as E. coli.

### Tangential Flow Filtration

Generally, the invention finds application in tangential-flow filtration technologies. For example, in one embodiment the invention relates to a process for preparing one or more target substances from a source liquid, the process comprising: contacting the source liquid with a population of biopolymer particles in or prior to addition to a tangential-flow filtration system, wherein one or more of the following steps are performed:

concentrating the population of polymer particles,

separating one or more contaminants from the one or more polymer particle-bound target substances or a polymer particle-bound precursor thereof, such as by diafiltration,

eluting the target substance from the polymer particles;

and recovering the target substance.

In embodiments relating to a particle-bound precursor of the target substance, one or more of the polymer particles comprises one or more enzymes capable of catalysing the conversion of the precursor to the target substance, or to a further precursor to the target substance. For example, in one embodiment the precursor of the target substance is a substrate of an enzyme capable of catalysing the conversion of the substrate to the target substance, and one or more of the polymer particle comprises the enzyme. In another example, the precursor of the target substance is a substrate of an enzyme capable of catalysing the conversion of the substrate to a further precursor to the target substance, which itself is the substrate of a second enzyme capable of catalysing the conversion of the further precursor to the target substance, and one or more of the polymer particles comprises the first enzyme, the second enzyme, or both the first and the second enzyme. It will be appreciated that by providing one or more polymer particles comprising appropriately chosen enzymes, a series of catalytic steps in the conversion of a precursor to the target substance can be employed.

In another embodiment the invention relates to a process for preparing one or more target substances from a source liquid, the process comprising: contacting the source liquid with a population of biopolymer particles in or prior to addition to a tangential-flow filtration system, wherein one or more of the following steps are performed:

concentrating the population of polymer particles,

separating one or more target substances or a precursor thereof from one or more polymer particle-bound contaminants, such as by diafiltration,

and recovering the target substance.

In one embodiment, the contacting the source liquid with a population of biopolymer particles is by circulating the biopolymer particles and the source liquid in a tangential-flow filtration system.

In another embodiment, the invention relates to a process for preparing one or more target substances from a source liquid, the process comprising adding to a tangential-flow filtration system a source liquid comprising a population of biopolymer particles and optionally one or more target substances or precursors thereof and/or one or more contaminants, and concentrating the population of polymer particles, and/or separating one or more of the target substances or precursors thereof and/or one or more of the contaminants from the polymer particles, and recovering the polymer particles, the target substance, or the contaminants.

The compositions, methods, and polymer particles of the invention have application in conjunction with existing tangential-flow systems and technologies. A great variety of such systems exist. In tangential-flow filtration systems, the shear force exerted on the filter element by the flow of the liquid medium tends to oppose the accumulation of solids on the surface of the filter element.

Tangential flow filters include microfiltration, ultrafiltration, nanofiltration and reverse osmosis filter systems. In one exemplary embodiment, the tangential-flow filter comprises a multiplicity of filter sheets (filtration membranes) in an operative stacked arrangement, e.g., wherein filter sheets alternate with permeate and retentate sheets, and as a liquid to be filtered flows across the filter sheets, impermeate species, e.g. solids or high-molecular-weight species of diameter larger than the filter sheet's pore size, are retained and enter the retentate flow, and the liquid along with any permeate species diffuse through the filter sheet and enter the permeate flow.

As will be appreciated, many tangential-flow technologies (also referred to as cross flow technologies) are currently available and are suitable for use in conjunction with the present invention. Commercially available tangential-flow technologies, including tangential-flow membrane filters, include, for example, the Vivaflow filters from Vivascience (including for example, the Vivaflow 200, 100,000 MWCO, PES, VivaScience), the Hydrosart® and polyethersulfone microfiltration and ultrafiltration membranes from Sartorius, while suitable tangential-flow filter modules and cassettes of such types are variously described in U.S. Pat. No. 4,867,876; U.S. Pat. No. 4,882,050; U.S. Pat. No. 5,034,124; U.S. Pat. No. 5,034,124; U.S. Pat. No. 5,049,268; U.S. Pat. No. 5,232,589; U.S. Pat. No. 5,342,517; U.S. Pat. No. 5,593,580; U.S. Pat. No. 5,868,930; and PCT International application PCT/US10/027,266, published as WO/2010/107677; the disclosures of all of which are hereby incorporated herein by reference in their respective entireties.

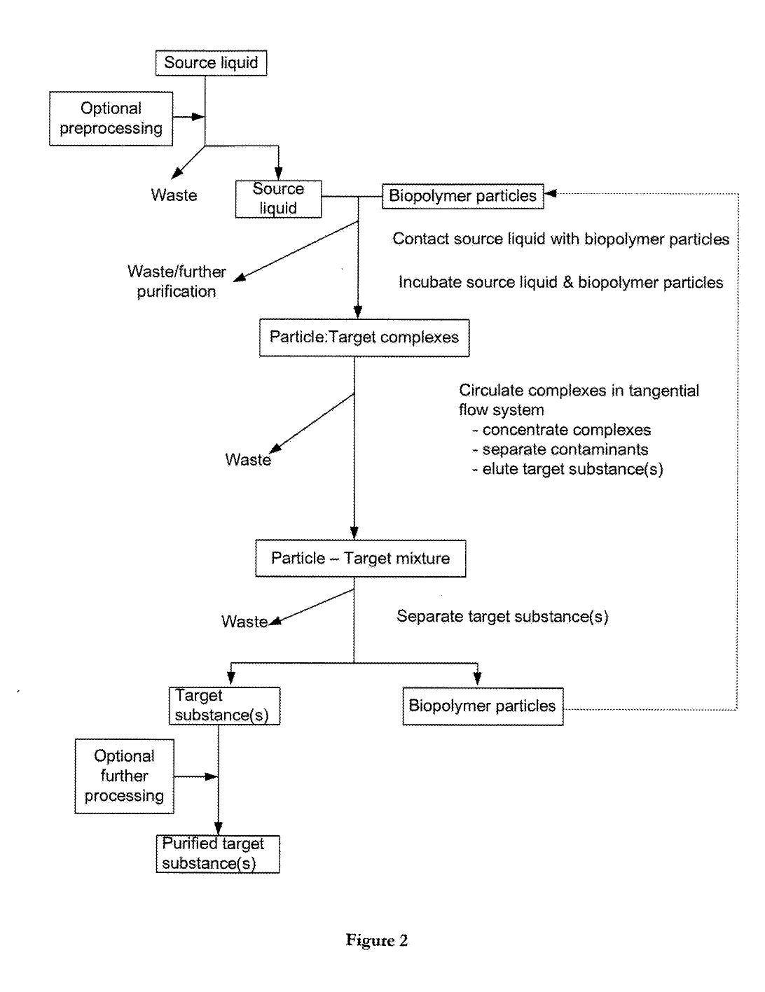

A general outline of exemplary tangential-flow processes applicable to the present invention are shown in FIGS. 1-4. Tangential Flow Filtration (TFF) or crossflow filtration (used interchangeably herein) is a process where filtration is achieved by running a solution or suspension flow path parallel to the surface of the filtration media (FIG. 1A). An exemplary, simple TFF system utilizes a feed pump to allow recirulation of the permeate in the system from a feed reservoir through the TFF membrane cartridge (FIG. 1B). Small compounds and solution pass through the filter as a permeate. In this way the large compounds in the retentate can be concentrated. By adding an additional solution reservoir the retentate can be dialyzed by filtration (diafiltered). Diafiltration in certain embodiments is therefore used as a process to separate large compounds (molecules, cells, solids in suspension) from smaller compounds. The use of TF filters in the submicron range e.g. 0.1-0.6 μm is commonly known as microfiltration. With reference to the general scheme shown in FIG. 2, the source liquid may optionally be preprocessed, for example, to remove particulate or solid matter (for example by centrifugation or filtration techniques well known in the art), concentrated, or diluted, as required for subsequent purification. The source liquid is then contacted with a population of biopolymer particles for a time sufficient to allow the formation of particle:target complexes.

Generally, the source liquid is contacted with the biopolymer particles for a time sufficient to lead to binding of a desirable proportion of the target(s) to the biopolymer particles. Mixing of the source liquid and biopolymer particles, for example, by stirring or processing through the tangential-flow filtration system will typically be advantageous to ensure optimal contact and binding of the target(s).

The particle:target complexes are circulated through a tangential-flow filtration system and thus through a tangential-flow filter where (a) the complexes are concentrated, (b) the complexes are diafiltered against a diafiltration liquid (typically selected to dissociate non-specifically bound contaminants from the particle:target complexes), (c) the target substance is eluted from the particles (typically by diafiltration with a second diafiltration liquid to dissociate the particle:target substance complexes, (d) the target substance is then separated (for example, the target substance is diafiltered away) from the biopolymer particles, thereby to recover purified target substance. The target substance may optionally be (e) further processed, for example by concentration.

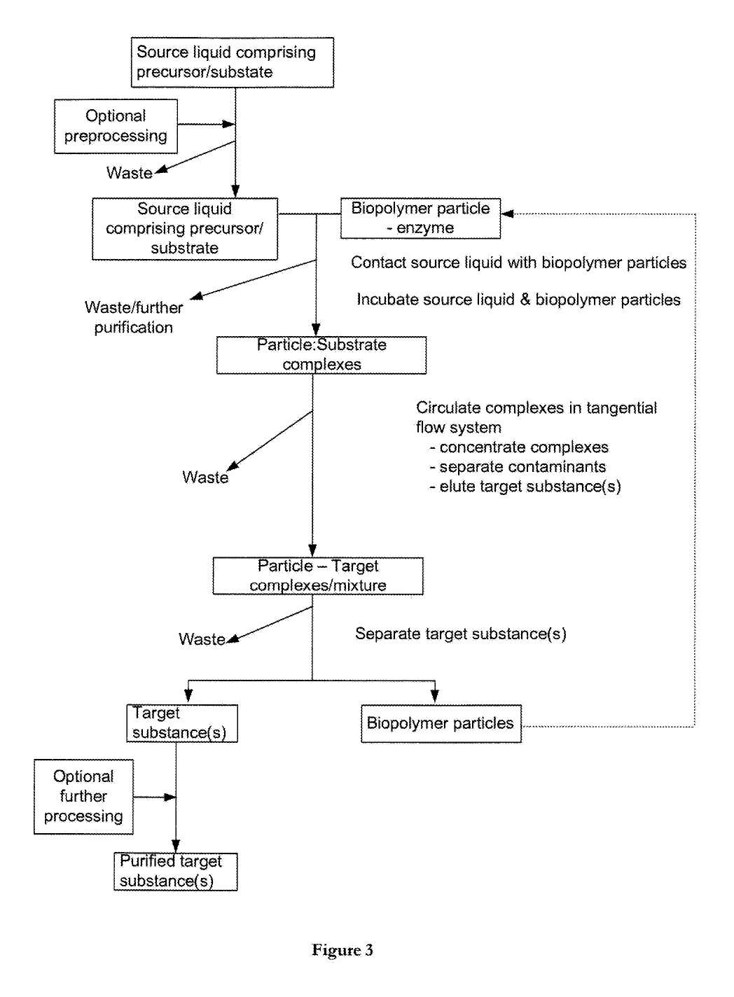

In a further exemplary embodiment depicted in FIG. 3, the source liquid comprises a precursor of the target substance, for example, a substrate of one more enzymes, the product of which is a desired target substance. In this embodiment, the source liquid comprising the precursor substance is contacted with one or more biopolymer particles comprising one or more enzymes capable of catalysing the conversion of the precursor to the target substance. As described above, the source liquid and biopolymer particles are contacted for a time sufficient to form complexes, albeit in this case a particle:enzyme-substrate complex. The source liquid and biopolymer particle mixture is maintained for a time sufficient to enable both a desirable proportion of precursor to be bound by the biopolymer particle, and to enable the conversion of the precursor molecule to the target substance.

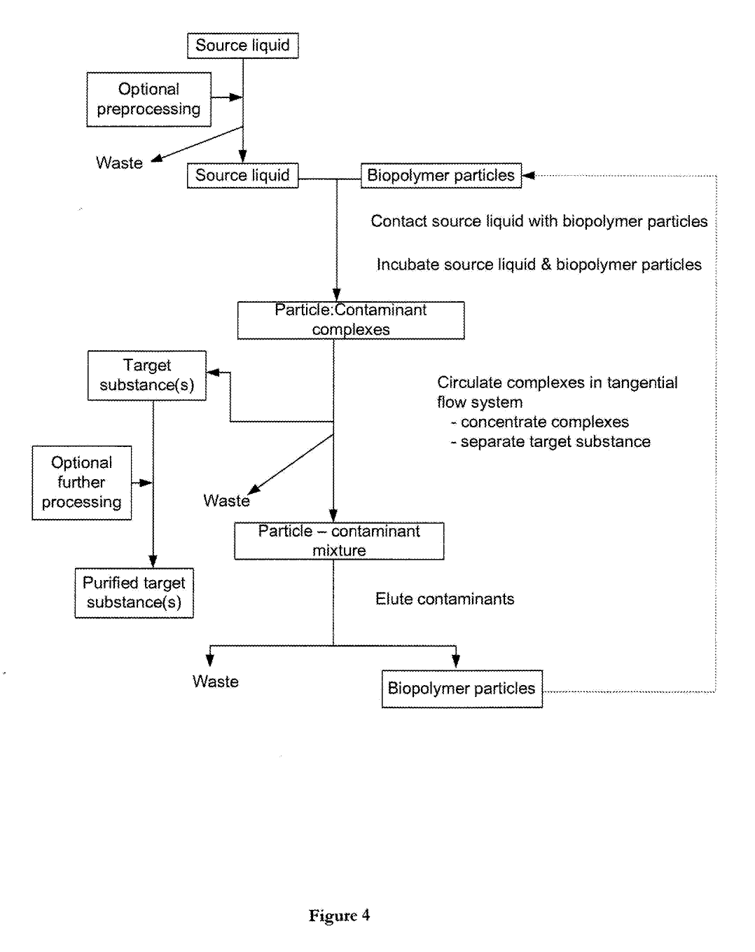

FIG. 4 shows a general scheme for the purification of a target substance using the methods of the invention wherein the biopolymer particles are used to enrich the target substance by removal of one or more contaminants. In this case, the source liquid is contacted with one or more populations of polymer particles capable of binding one or more contaminants present in the source liquid. Here, the formation of particle:contaminant complexes allows the diafiltration of target substance(s) which may then be further processed (including for example, via one or more tangential-flow methods of the present invention as described herein). Diafiltration (typically with a second diafiltration liquid) allows dissociation with the particle:contaminant complexes, wherein the biopolymer particles may be recirculated for further use.

Accordingly, those skilled in the art will recognise that the various embodiments of the invention (including those representative examples outlined above) contemplate the elution of desired target substances from the tangential-flow filtration system in either the retentate or in the permeate, depending on the particular configuration or the particular population of polymer particles used.

### Source Materials

The present invention relates to the preparation of a target substance from a source material. A “source material” as used herein refers to a material, typically a liquid, containing at least one and frequently more than one substance, usually a biological substance, or product of value which are sought to be extracted or purified from other substances present in the source material. Generally, source materials may for example be aqueous solutions, organic solvent systems, or aqueous/organic solvent mixtures or solutions. The source materials are often complex mixtures or solutions containing many biological molecules such as proteins, antibodies, hormones, and viruses as well as small molecules such as salts, sugars, lipids, and the like. While a typical source material of biological origin may begin as an aqueous solution or suspension, it may also contain organic solvents used in earlier separation steps such as solvent precipitations, extractions, and the like. Examples of source liquids that may contain valuable biological substances amenable to the purification method of the invention include, but are not limited to, a culture supernatant from a bioreactor, a homogenized cell suspension, plasma, plasma fractions, and dairy processing streams such as milk, colostrum and whey such as cheese whey.

In various embodiments, the source material comprises one or more liquids selected from the group consisting of serum, plasma, plasma fractions, whole blood, milk, colostrum, whey, cell fluids, tissue culture fluids, plant cells fluids, plant cell homogenates, and tissue homogenates. For example, the source material is a plant extract, such as a fruit juice or a vegetable juice. Fermentates are particularly contemplated, as are cultures or culture supernatants, particularly those of cultures expressing one or more recombinant proteins, such as one or more monoclonal antibodies.

In other embodiments, the source material comprises culture supernatants, for example from a bioreactor, comprising polymer particles of the invention. Those skilled in the art will recognise that such source material comprises a significant amount of other biological materials which in certain circumstances may be considered contaminants. For example, in one exemplary embodiment, the source material is a culture supernatant or cell preparation comprising bacteria used to produce the polymer particles of the present invention. In another expressly contemplated embodiment, the source material is a culture supernatant or cell preparation from cells producing the polymer particles of the present invention and cells producing one or more target substances or precursors thereof. In certain embodiments, the source material is a culture supernatant or cell preparation comprising a population of cells producing both polymer particles of the present invention and one or more target substances or precursors thereof.

### Target Substances

As above, the present invention relates to the preparation of a target substances from source materials, including source materials comprising a precursor of the target substance. The term “target substance” as used herein refers to the one or more desired product or products to be prepared or purified from the source liquid. Target substances are typically biological products of value, for example, immunoglobulins, clotting factors, vaccines, antigens, antibodies, selected proteins or glycoproteins, peptides, enzymes, metabolites, and the like.

In various embodiments, the target substance is selected from the group consisting of vaccines, clotting factors, immunoglobulins, antigens, antibodies, proteins, glycoproteins, peptides, sugars, carbohydrates, and enzymes.

In various embodiments, the one or more affinity ligands bind at least one of the target species selected from the group consisting of proteins, nucleic acids, viruses, sugars, carbohydrates, immunoglobulins, clotting factors, glycoproteins, peptides, antibodies, antigens, hormones, or polynucleotides.

However, the invention finds application in the preparation of a wide variety of target substances other than those typically considered to be ‘biological’, as will be appreciated on recognition of the multiplicity of functional moieties which may be associated with the polymer particles described herein. For example, the polymer particles of the invention may be conveniently functionalised with metal or metal-ion binding moieties, such as metal or metal-ion co-ordinating polypeptides, for example by expression of a polymer synthase:metal-binding polypeptide fusion polypeptide. Indeed, the ability to fuse one or more protein functionalities to the polymer-forming protein or polymer-binding protein comprising the polymer particles allows for application in the preparation of an extremely varied range of target substances.

The fusion polypeptides comprising the biopolymer particles of the invention may conveniently be produced using biotechnological techniques well known in the art, including the use of one or more expression constructs. It will be appreciated that in certain embodiments the fusion polypeptides comprising the biopolymer particles of the invention and the biopolymer particles are themselves a target substance as contemplated herein.

### Expression Constructs

Processes for producing and using expression constructs for expression of fusion polypeptides in microorganisms, plant cells or animal cells (cellular expression systems) or in cell free expression systems, and host cells comprising expression constructs useful for forming polymer particles for use in the invention are well known in the art (e.g. Sambrook et al., 1987; Ausubel et al., 1987).

Expression constructs for use in methods of the invention are in one embodiment inserted into a replicable vector for cloning or for expression, or in another embodiment are incorporated into the host genome. Various vectors are publicly available. The vector is, for example, in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more selectable marker genes, an enhancer element, a promoter, and a transcription termination sequence. Construction of suitable vectors containing one or more of these components employs standard ligation techniques known in the art.

Both expression and cloning vectors contain a nucleic acid sequence that enables the vector to replicate in one or more selected host cells. Such sequences are well known for a variety of bacteria, yeast, and viruses.

In one embodiment the expression construct is present on a high copy number vector.

In one embodiment the high copy number vector is selected from those that are present at 20 to 3000 copies per host cell.

In one embodiment the high copy number vector contain a high copy number origin of replication (ori), such as ColE1 or a ColE1-derived origin of replication. For example, the ColE-1 derived origin of replication may comprise the pUC19 origin of replication.

Numerous high copy number origins of replication suitable for use in the vectors of the present invention are known to those skilled in the art. These include the ColE1-derived origin of replication from pBR322 and its derivatives as well as other high copy number origins of replication, such as M13 FR ori or p15A ori. The 2μ plasmid origin is suitable for yeast, and various viral origins (SV40, polyoma, adenovirus, VSV or BPV) are useful for cloning vectors in mammalian cells.

Preferably, the high copy number origin of replication comprises the ColE1-derived pUC19 origin of replication.

The restriction site is positioned in the origin of replication such that cloning of an insert into the restriction site will inactivate the origin, rendering it incapable of directing replication of the vector. Alternatively, the at least one restriction site is positioned within the origin such that cloning of an insert into the restriction site will render it capable of supporting only low or single copy number replication of the vector.

Expression and cloning vectors will typically contain a selection gene, also termed a selectable marker to detect the presence of the vector in the transformed host cell. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Examples of suitable selectable markers for mammalian cells are those that enable the identification of cells competent to take up expression constructs, such as DHFR or thymidine kinase. An appropriate host cell when wild-type DHFR is employed is the CHO cell line deficient in DHFR activity, prepared and propagated as described by Urlaub et al., 1980. A suitable selection gene for use in yeast is the trp1 gene present in the yeast plasmid YRp7 (Stinchcomb et al., 1979; Kingsman et al., 1979; Tschemper et al., 1980). The trp1 gene provides a selection marker for a mutant strain of yeast lacking the ability to grow in tryptophan, for example, ATCC No. 44076 or PEP4-1 [Jones, Genetics, 85:12 (1977)].

An expression construct useful for forming polymer particles preferably includes a promoter which controls expression of at least one nucleic acid encoding a polymer synthase, particle-forming protein or fusion polypeptide.

Promoters recognized by a variety of potential host cells are well known. Promoters suitable for use with prokaryotic hosts include the β-lactamase and lactose promoter systems (Chang et al., 1978; Goeddel et al., 1979), alkaline phosphatase, a tryptophan (trp) promoter system (Goeddel, Nucleic Acids Res., 8:4057 (1980); EP 36,776), and hybrid promoters such as the tac promoter (deBoer et al., 1983). Promoters for use in bacterial systems also will contain a Shine-Dalgarno (S.D.) sequence operably linked to the nucleic acid encoding a polymer synthase, particle-forming protein or fusion polypeptide.

Examples of suitable promoting sequences for use with yeast hosts include the promoters for 3-phosphoglycerate kinase (Hitzeman et al., 1980) or other glycolytic enzymes (Hess et al., 1968; Holland, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Examples of suitable promoters for use in plant host cells, including tissue or organ of a monocot or dicot plant include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters are those from the host cell, or promoters which are derived from genes of other plants, viruses, and plant pathogenic bacteria and fungi. Those skilled in the art will, without undue experimentation, be able to select promoters that are suitable for use in modifying and modulating expression constructs using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Examples of suitable promoters for use in mammalian host cells comprise those obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of an expression construct by higher eukaryotes is in some examples increased by inserting an enhancer sequence into the vector. Enhancers are cis-acting elements of DNA, usually about from 10 to 300 bp that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. Typically, the enhancer is spliced into the vector at a position 5′ or 3′ to the polymer synthase, particle-forming protein or fusion polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polymer synthase, particle-forming protein or fusion polypeptide.

In one embodiment the expression construct comprises an upstream inducible promoter, such as a BAD promoter, which is induced by arabinose.

In one embodiment the expression construct comprises a constitutive or regulatable promoter system.

In one embodiment the regulatable promoter system is an inducible or repressible promoter system.

While it is desirable to use strong promoters in the production of recombinant proteins, regulation of these promoters is essential since constitutive overproduction of heterologous proteins leads to decreases in growth rate, plasmid stability and culture viability.

A number of promoters are regulated by the interaction of a repressor protein with the operator (a region downstream from the promoter). The most well known operators are those from the lac operon and from bacteriophage A. An overview of regulated promoters in E. coli is provided in Table 1 of Friehs & Reardon, 1991.

A major difference between standard bacterial cultivations and those involving recombinant E. coli is the separation of the growth and production or induction phases. Recombinant protein production often takes advantage of regulated promoters to achieve high cell densities in the growth phase (when the promoter is “off” and the metabolic burden on the host cell is slight) and then high rates of heterologous protein production in the induction phase (following induction to turn the promoter “on”).

In one embodiment the regulatable promoter system is selected from LacI, Trp, phage γ and phage RNA polymerase.

In one embodiment the promoter system is selected from the lac or Ptac promoter and the lad repressor, or the trp promoter and the TrpR repressor.

In one embodiment the Lad repressor is inactivated by addition of isopropyl-β-D-thiogalactopyranoside (IPTG) which binds to the active repressor causes dissociation from the operator, allowing expression.

In one embodiment the trp promoter system uses a synthetic media with a defined tryptophan concentration, such that when the concentration falls below a threshold level the system becomes self-inducible. In one embodiment 3-β-indole-acrylic acid is added to inactivate the TrpR repressor.

In one embodiment the promoter system may make use of the bacteriophage γ repressor cI. This repressor makes use of the γ prophage and prevent expression of all the lytic genes by interacting with two operators termed OL and OR. These operators overlap with two strong promoters PL and PR respectively. In the presence of the cI repressor, binding of RNA polymerase is prevented. The cI repressor can be inactivated by UV-irradiation or treatment of the cells with mitomycin C. A more convenient way to allow expression of the recombinant polypeptide is the application of a temperature-sensitive version of the cI repressor cI857. Host cells carrying a γ-based expression system can be grown to mid-exponential phase at low temperature and then transferred to high temperature to induce expression of the recombinant polypeptide.

A widely used expression system makes use of the phage T7 RNA polymerase which recognises only promoters found on the T7 DNA, and not promoters present on the host cell chromosome. Therefore, the expression construct may contain one of the T7 promoters (normally the promoter present in front of gene 10) to which the recombinant gene will be fused. The gene coding for the T7 RNA polymerase is either present on the expression construct, on a second compatible expression construct or integrated into the host cell chromosome. In all three cases, the gene is fused to an inducible promoter allowing its transcription and translation during the expression phase.

The E. coli strains BL21 (DE3) and BL21 (DE3) pLysS (Invitrogen, CA) are examples of host cells carrying the T7 RNA polymerase gene (there are a few more very suitable and commercially available E. coli strains harbouring the T7RNA polymerase gene such as e.g. KRX and XJ (autolysing)). Other cell strains carrying the T7 RNA polymerase gene are known in the art, such as Pseudomonas aeruginosa ADD1976 harboring the T7 RNA polymerase gene integrated into the genome (Brunschwig & Darzins, 1992) and Cupriavidus necator (formerly Ralstonia eutropha) harboring the T7 RNA polymerase gene integrated into the genome under phaP promoter control (Barnard et al., 2004).

The T7 RNA polymerase offers three advantages over the host cell enzymes: First, it consists of only one subunit, second it exerts a higher processivity, and third it is insensitive towards rifampicin. The latter characteristic can be used especially to enhance the amount of fusion polypeptide by adding this antibiotic about 10 min after induction of the gene coding for the T7 RNA polymerase. During that time, enough polymerase has been synthesised to allow high-level expression of the fusion polypeptide, and inhibition of the host cell enzymes prevents further expression of all the other genes present on both the plasmid and the chromosome. Other antibiotics which inhibit the bacterial RNA polymerase but not the T7 RNA polymerase are known in the art, such as streptolydigin and streptovaricin.

Since all promoter systems are leaky, low-level expression of the gene coding for T7 RNA polymerase may be deleterious to the cell in those cases where the recombinant polypeptide encodes a toxic protein. These polymerase molecules present during the growth phase can be inhibited by expressing the T7-encoded gene for lysozyme. This enzyme is a bifunctional protein that cuts a bond in the cell wall of the host cell and selectively inhibits the T7 RNA polymerase by binding to it, a feed-back mechanism that ensures a controlled burst of transcription during T7 infection. The E. coli strain BL21 (DE3) pLysS is an example of a host cell that carries the plasmid pLysS that constitutively expresses T7 lysozyme.

In one embodiment the promoter system makes use of promoters such as API or APR which are induced or “switched on” to initiate the induction cycle by a temperature shift, such as by elevating the temperature from about 30-37° C. to 42° C. to initiate the induction cycle.

A strong promoter may enhance fusion polypeptide density at the surface of the particle during in-vivo production.

Preferred fusion polypeptides for use in one embodiment of the present invention comprise a (i) a polymer synthase and (ii) a fusion partner comprising at least one antibody binding domain

A nucleic acid sequence encoding both (i) and (ii) for use herein comprises a nucleic acid encoding a polymer synthase and a nucleic acid encoding a fusion partner comprising at least one antibody binding domain Once expressed, the fusion polypeptide is able to form or facilitate formation of a polymer particle.

In one embodiment the nucleic acid sequence encoding at least polymer synthase is indirectly fused with the nucleic acid sequence encoding a particle-forming protein or the nucleic acid encoding a fusion partner through a polynucleotide linker or spacer sequence of a desired length.

In one embodiment the amino acid sequence of the fusion polypeptide encoding at least one fusion partner is contiguous with the C-terminus of the amino acid sequence comprising a polymer synthase.

In one embodiment the amino acid sequence of the fusion protein comprising at least one fusion partner is indirectly fused with the N-terminus of the amino acid sequence comprising a polymer synthase fragment through a peptide linker or spacer of a desired length that facilitates independent folding of the fusion polypeptides.

In one embodiment the amino acid sequence of the fusion polypeptide encoding at least one fusion partner is contiguous with the N-terminus of the amino acid sequence comprising a particle-forming protein or a C-terminal synthase fragment.

In one embodiment the amino acid sequence of the fusion protein encoding at least one fusion partner is indirectly fused with the C-terminus of the amino acid sequence comprising a particle-forming protein or a N-terminal polymer synthase fragment through a peptide linker or spacer of a desired length to facilitate independent folding of the fusion polypeptides.

In one embodiment the amino acid sequence of the fusion polypeptide encoding at least one fusion partner is contiguous with the N-terminus of the amino acid sequence encoding a depolymerase, or a C-terminal depolymerase fragment.

One advantage of the fusion polypeptides according to the present invention is that the modification of the proteins binding to the surface of the polymer particles does not affect the functionality of the proteins involved in the formation of the polymer particles. For example, the functionality of the polymer synthase is retained if a recombinant polypeptide is fused with the N-terminal end thereof, resulting in the production of recombinant polypeptide on the surface of the particle. Should the functionality of a protein nevertheless be impaired by the fusion, this shortcoming is offset by the presence of an additional particle-forming protein which performs the same function and is present in an active state.

In this manner, it is possible to ensure a stable bond of the recombinant polypeptide bound to the polymer particles via the particle-forming proteins.

It should be appreciated that the arrangement of the proteins in the fusion polypeptide is dependent on the order of gene sequences in the nucleic acid contained in the plasmid.

For example, it may be desired to produce a fusion polypeptide wherein the fusion partner is indirectly fused to the polymer synthase. The term “indirectly fused” refers to a fusion polypeptide comprising a particle-forming protein, preferably a polymer synthase, and at least one fusion partner that are separated by an additional protein which may be any protein that is desired to be expressed in the fusion polypeptide.

In one embodiment the additional protein is selected from a particle-forming protein or a fusion polypeptide, or a linker or spacer to facilitate independent folding of the fusion polypeptides, as discussed above. In this embodiment it would be necessary to order the sequence of genes in the plasmid to reflect the desired arrangement of the fusion polypeptide.

In one embodiment the fusion partner is directly fused to the polymer synthase. The term “directly fused” is used herein to indicate where two or more peptides are linked via peptide bonds.

It may also be possible to form a particle wherein the particle comprises at least two distinct fusion polypeptides that are bound to the polymer particle. For example, a first fusion polypeptide comprising a binding domain capable of binding at least one enzyme product fused to a polymer synthase could be bound to the polymer particle, and a second fusion polypeptide comprising the enzyme could be bound to the polymer particle.

In one embodiment the expression construct is expressed in vivo. Preferably the expression construct is a plasmid which is expressed in a microorganism, preferably Escherichia

In one embodiment the expression construct is expressed in vitro. Preferably the expression construct is expressed in vitro using a cell-free expression system.

In one embodiment one or more genes can be inserted into a single expression construct, or one or more genes can be integrated into the host cell genome. In all cases expression can be controlled through promoters as described above.

In one embodiment the expression construct further encodes at least one additional fusion polypeptide comprising an antigen capable of eliciting a cell-mediated immune response or a binding domain capable of binding at least one antigen capable of eliciting a cell-mediated immune response and a particle-forming protein, preferably a polymer synthase, as discussed above.

Plasmids useful herein are shown in the examples and are described in detail in PCT/DE2003/002799 published as WO 2004/020623 (Bernd Rehm) and PCT/NZ2006/000251 published as WO 2007/037706 (Bernd Rehm) which are each herein incorporated by reference in their entirety.

### Hosts for Particle Production

The particles of the present invention are conveniently produced in a host cell, using one or more expression constructs as herein described. Polymer particles of the invention can be produced by enabling the host cell to express the expression construct. This can be achieved by first introducing the expression construct into the host cell or a progenitor of the host cell, for example by transforming or transfecting a host cell or a progenitor of the host cell with the expression construct, or by otherwise ensuring the expression construct is present in the host cell.

Following transformation, the transformed host cell is maintained under conditions suitable for expression of the fusion polypeptides from the expression constructs and for formation of polymer particles. Such conditions comprise those suitable for expression of the chosen expression construct, such as a plasmid in a suitable organism, as are known in the art. For example, and particularly when high yield or overexpression is desired, provision of a suitable substrate in the culture media allows the particle-forming protein component of a fusion polypeptide to form a polymer particle.

Preferably the host cell is, for example, a bacterial cell, a fungi cell, yeast cell, a plant cell, an insect cell or an animal cell, preferably an isolated or non-human host cell. Host cells useful in methods well known in the art (e.g. Sambrook et al., 1987; Ausubel et al., 1987) for the production of recombinant polymer particles are frequently suitable for use in the methods of the present invention, bearing in mind the considerations discussed herein.

Suitable prokaryote host cells comprise, for example, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include other Enterobacteriaceae such as Escherichia spp., Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Actinomycetes such as Streptomyces, Rhodococcus, Corynebacterium and Mycobaterium.

In some embodiments, for example, E. coli strain W3110 may be used because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF-lac)169 degP ompT rbs7 ilvG kanr; E. coli W3110 strain 40B4, which is strain 37D6 with a non-kanamycin resistant degP deletion mutation.

In some preferred embodiments, for example, Lactococcus lactic strains that do not produce lipopolysaccharide endotoxins may be used. Examples of Lactococcus lactis strains include MG1363 and Lactococcus lactic subspecies cremoris NZ9000.

In addition to prokaryotes, eukaryotic microbes such as filamentous fungi or yeast are suitable cloning or expression hosts for use in the methods of the invention, for example. Examples include Saccharomyces cerevisiae, a commonly used lower eukaryotic host microorganism. Other examples include Schizosaccharomyces pombe (Beach and Nurse, 1981; EP 139,383), Kluyveromyces hosts (U.S. Pat. No. 4,943,529; Fleer et al., 1991) such as, e.g., K. lactis (MW98-8C, CBS683, CBS4574; Louvencourt et al., 1983), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al, 1990), K. thermotolerans, and K. marxianus; yarrowia (EP 402,226); Pichia pastoris (EP 183,070; Sreekrishna et al., 1988); Candida; Trichoderma reesia (EP 244,234); Neurospora crassa (Case et al., 1979); Schwanniomyces such as Schwanniomyces occidentalis (EP 394,538 published 31 Oct. 1990); and filamentous fungi such as, e.g., Neurospora, Penicillium Tolypocladium (WO 91/00357 published 10 Jan. 1991), and Aspergillus hosts such as A. nidulans (Ballance et al., 1983; Tilburn et al., 1983; Yelton et al., 1984) and A. niger (Kelly and Hynes, 1985). Methylotropic yeasts are suitable herein and comprise yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in Anthony, 1982.

Examples of invertebrate host cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa califormica NPV and the Bm-5 strain of Bombyx mori NPV, and such viruses may be used as the virus herein according to the present invention, particularly for transfection of Spodoptera frugiperda cells.

Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., 1980); mouse sertoli cells (TM4, Mather, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982); MRC 5 cells; FS4 cells; and a human hepatoma line (Hep G2).

Eukaryotic cell lines, for example mammalian cell lines, will be preferred when, for example, the fusion partner, such as an enzyme or an antibody binding domain requires one or more post-translational modifications, such as, for example, glycation. For example, one or more enzymes may require post-translational modification to be optimally active, and may thus be usefully expressed in an expression host capable of such post-translational modifications.

In one embodiment the host cell is a cell with an oxidising cytosol, for example the E. coli Origami strain (Novagen).

In another embodiment the host cell is a cell with a reducing cytosol, preferably E. coli.

The host cell, for example, may be selected from the genera comprising Ralstonia, Acaligenes, Pseudomonas and Halobiforma. Preferably the microorganism used is selected from the group comprising, for example, Ralstonia eutropha, Alcaligenes latus, Escherichia coli, Pseudomonas fragi, Pseudomonas putida, Pseudomonas oleovorans, Pseudomonas aeruginosa, Pseudomonas fluorescens, and Halobiforma haloterrestris. This group comprises both microorganisms which are naturally capable of producing biocompatible, biodegradable particles and microorganisms, such as for example E. coli, which, due to their genetic makeup, are not capable of so doing. The genes required to enable the latter-stated microorganisms to produce the particles are introduced as described above.

Extremely halophilic archaea produce polymer particles with lower levels of unspecific binding of protein, allowing easier isolation and purification of the particles from the cells.

In principle, any culturable host cell may be used for the production of polymer particles by means of the above-described process, even if the host cell cannot produce the substrates required to form the polymer particles due to a different metabolism. In such cases, the necessary substrates are added to the culture medium and are then converted into polymer particle by the proteins which have been expressed by the genes which have been introduced into the cell.

Genes utilized to enable the latter-stated host cells to produce the polymer particles include, for example, a thiolase, a reductase or a polymer synthase, such as phaA thiolase, phaB ketoacyl reductase or phaC synthase from Ralstonia eutropha. Which genes are used to augment what the host cell lacks for polymer particle formation will be dependent on the genetic makeup of the host cell and which substrates are provided in the culture medium.

The genes and proteins involved in the formation of polyhydroxyalkanoate (PHA) particles, and general considerations for particle formation are reported in Madison, et al, 1999; published PCT International Application WO 2004/020623 (Bernd Rehm); and Rehm, 2003; Brockelbank J A. et al., 2006; Peters and Rehm, 2006; Bäckström et al, (2006) and Rehm, (2006), all of which are herein incorporated by reference.

A polymer synthase alone can be used in any host cell with (R)-Hydroxyacyl-CoA or other CoA thioester or derivatives thereof as a substrate.

The polymer particle can also be formed in vitro. Preferably, for example, a cell free expression system is used. In such systems a polymer synthase is provided. Purified polymer synthase, such as that obtainable from recombinant production, or in cell free systems capable of protein translation, that obtainable in the cell free system itself by way of introduction of an expression construct encoding a polymer synthase, will be preferred. In order to produce an environment to allow particle formation in vitro the necessary substrates for polymer particle formation should be included in the media.

The polymer synthase can be used for the in vitro production of functionalised polymer particles using (R)-Hydroxyacyl-CoA or other CoA thioester as a substrate, for example.

The fusion polypeptides can be purified from lysed cells using a cell sorter, centrifugation, filtration or affinity chromatography prior to use in in vitro polymer particle production.

In vitro polymer particle formation enables optimum control of surface composition, including the level of fusion polypeptide coverage, phospholipid composition and so forth.

It will be appreciated that the characteristics of the polymer particle may be influenced or controlled by controlling the conditions in which the polymer particle is produced. This may include, for example, the genetic make-up of the host cell, eg cell division mutants that produce large granules, as discussed in Peters and Rehm, 2005. The conditions in which a host cell is maintained, for example temperature, the presence of substrate, the presence of one or more particle-forming proteins such as a particle size-determining protein, the presence of a polymer regulator, and the like.

In one embodiment, a desirable characteristic of the polymer particle is that it is persistent. The term “persistent” refers to the ability of the polymer particle to resist degradation in a selected environment. An additional desirable characteristic of the polymer particle is that it is formed from the polymer synthase or particle-forming protein and binds to the C- or N-terminal of the polymer synthase or particle-forming protein during particle assembly.

In some embodiments of the invention it is desirable to achieve overexpression of the expression constructs in the host cell. Mechanisms for overexpression a particular expression construct are well known in the art, and will depend on the construct itself, the host in which it is to be expressed, and other factors including the degree of overexpression desired or required. For example, overexpression can be achieved by i) use of a strong promoter system, for example the T7 RNA polymerase promoter system in prokaryotic hosts; ii) use of a high copy number plasmid, for example a plasmid containing the colE1 origin of replication or iii) stabilisation of the messenger RNA, for example through use of fusion sequences, or iv) optimization of translation through, for example, optimization of codon usage, of ribosomal binding sites, or termination sites, and the like. The benefits of overexpression may allow the production of smaller particles where desired and the production of a higher number of polymer particles.

The composition of the polymers forming the polymer particles may affect the mechanical or physiochemical properties of the polymer particles. For example, polymer particles differing in their polymer composition may differ in half-life or may release biologically active substances, in particular pharmaceutical active ingredients, at different rates. For example, polymer particles composed of C₆-C₁₄ 3-hydroxy fatty acids exhibit a higher rate of polymer degradation due to the low crystallinity of the polymer. An increase in the molar ratio of polymer constituents with relatively large side chains on the polymer backbone usually reduces crystallinity and results in more pronounced elastomeric properties. By controlling polymer composition in accordance with the process described herein and known in the art, it is accordingly possible to influence the biodegradability of the polymer particles and thus affect the duration the polymer particles (and when present the one or more fusion partners are maintained in, for example, a tangential-flow filtration system, or to affect the binding, catalysis, or release of one or more target substances or precursors thereof to, on, or from the polymer particles.

At least one fatty acid with functional side groups is preferably introduced into the culture medium as a substrate for the formation of the polymer particles, with at least one hydroxy fatty acid and/or at least one mercapto fatty acid and/or at least one β-amino fatty acid particularly preferably being introduced. “Fatty acids with functional side groups” should be taken to mean saturated or unsaturated fatty acids. These also include fatty acids containing functional side groups which are selected from the group comprising methyl groups, alkyl groups, hydroxyl groups, phenyl groups, sulfhydryl groups, primary, secondary and tertiary amino groups, aldehyde groups, keto groups, ether groups, carboxyl groups, O-ester groups, thioester groups, carboxylic acid amide groups, hemiacetal groups, acetal groups, phosphate monoester groups and phosphate diester groups. Use of the substrates is determined by the desired composition and the desired properties of the polymer particle.

The substrate or the substrate mixture may comprise at least one optionally substituted amino acid, lactate, ester or saturated or unsaturated fatty acid, preferably acetyl-CoA.

In one embodiment one or more substances is provided in the substrate mixture and is incorporated into the polymer particle during polymer particle formation, or is allowed to diffuse into the polymer particle.

The polymer particle may comprise a polymer selected from poly-beta-hydroxy acids, polylactates, polythioesters and polyesters, for example. Most preferably the polymer comprises polyhydroxyalkanoate (PHA), preferably poly(3-hydroxybutyrate) (PHB).

The polymer synthase or polymer particle preferably comprises a phospholipid monolayer that encapsulates the polymer particle. Preferably said particle-forming protein spans said lipid monolayer.

The polymer synthase or particle-forming protein is preferably bound to the polymer particle or to the phospholipid monolayer or is bound to both.

The particle-forming protein is preferably covalently or non-covalently bound to the polymer particle it forms.

Preferably at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or 100% of the surface area of the polymer particle is covered by fusion polypeptides.

In certain circumstances it may be desirable to control the size of the particles produced in the methods of the invention, for example to produce particles particularly suited to a given application. For example, it may be desirable to produce polymer particles comprising one or more fusion partners at a relatively large size, for example to support robust durability. For example, in the context of particles for use in the preparation of one or more antibodies, it may be desirable to produce polymer particles comprising one or more antibody binding domains of a relatively large size to ensure durability and functionality in tangential-flow filtration systems. In other examples, such as in the catalysis of an enzyme substrate to a target substance, it may be desirable to produce polymer particles comprising one or more enzymes of a relatively small size, for example to enable a high relative concentration of enzyme in the tangential-flow filtration system. Methods to control the size of polymer particles are described in PCT/DE2003/002799 published as WO 2004/020623, and PCT/NZ2006/000251 published as WO 2007/037706.

In some embodiments, particle size is controlled by controlling the expression of the particle-forming protein, or by controlling the expression of a particle size-determining protein if present, for example.

In other embodiments of the present invention, for example, particle size control may be achieved by controlling the availability of a substrate, for example the availability of a substrate in the culture medium. In certain examples, the substrate may be added to the culture medium in a quantity such that it is sufficient to ensure control of the size of the polymer particle.

It will be appreciated that a combination of such methods may be used, allowing the possibility for exerting still more effective control over particle size.

In various embodiments, for example, particle size may be controlled to produce particles having a diameter of from about 10 nm to 3 μm, preferably from about 10 nm to about 900 nm, from about 10 nm to about 800 nm, from about 10 nm to about 700 nm, from about 10 nm to about 600 nm, from about 10 nm to about 500 nm, from about 10 nm to about 400 nm, from about 10 nm to about 300 nm, from about 10 nm to about 200 nm, and particularly preferably of from about 10 nm to about 100 nm

In other embodiments, for example, particle size may be controlled to produce particles having a diameter of from about 10 nm to about 90 nm, from about 10 nm to about 80 nm, from about 10 nm to about 70 nm, from about 10 nm to about 60 nm, from about 10 nm to about 50 nm, from about 10 nm to about 40 nm, from about 10 nm to about 30 nm, or from about 10 nm to about 20 nm

Other ranges of average polymer size, for example, including ranges within the above recited ranges, are specifically contemplated, for example polymer particles having a diameter of from about 50 to about 500 nm, from about 150 to about 250 nm, or from about 100 to about 500 nm, etc.

In various embodiments, for example, 90% of the particles produced have a diameter of about 200 nm or below, 80% have a diameter about 150 nm or below, 60% have a diameter about 100 nm or below, 45% have a diameter about 80 nm or below, 40% have a diameter about 60 nm or below, 25% have a diameter about 50 nm or below, and 5% have a diameter about 35 nm or below

In various embodiments, for example, the method produces polymer particles with an average diameter less than about 200 nm, less than about 150 nm, or less than about 110 nm.

The invention consists in the foregoing and also envisages constructions of which the following gives examples only.

### EXAMPLES

### Example 1

### Purification of IgG by Tangential-Flow Filtration

This example describes the use of polymer particles presenting an antibody-binding polypeptide domain in conjunction with various commercially-available tangential-flow membranes to purify IgG immunoglobulins.

### Materials and Methods

All filtrations were conducted with the Sartorius Crossflow Slice 200 system. The following membranes were used: 0.1 μm Polyethersulfone, 0.2 μm Hydrosart, and 100 kDa Hydrosart, each from Sartorius.

### Pressure Monitoring

P1, P2 and P3 are connected to a pressure transducer that records and send signals to the central controller from each point. P1, P2 and P3 are initially controlled by clamping of the tubing, and were left untouched throughout a run (Water flux and Diafiltration). Hence, trans-membrane pressure (TMP) was maintained by automated variation of pump feed rate.

### Particle Preparation

ZZPhaC polymer particles were prepared in a bioreactor by culturing E. coli BL21 bacteria carrying pET14b-ZZ(−)phaC plasmid. (Brockelbank et al., 2006). All biomass was lysed with BugBuster protocol and washed 3 times in 50 mM Potassium phosphate buffer, pH 7.5.

2.69 g of polymer particles was resuspended in 50 mL of 50 mM Potassium phosphate buffer, pH 7.5, resulting in 53.8 g/L of polymer particle suspension.

15 mL of 53.8 g/L polymer particle suspension was made up to 100 mL with 50 mM Phosphate buffer, pH 7.5, resulting in 8.07 g/L polymer particle suspension.

This 100 mL of 8.07 g/L polymer particle suspension was used for diafiltration during the tangential-flow filtration.

### Clear Water Flux

Clear water flux was done before and after diafiltration of polymer particle suspension to monitor the filter membrane quality.

### 0.1 μm Filter:—

Before diafiltration, TMP=1 bar, Permeate flow=272 mL/min
After diafiltration, membrane was disinfected with 1M NaOH (40° C.) for 15 min, then again for 30 min, and rinsed with MQ water. 0.1M NaOH was circulated in the membrane for storage.
TMP=1 bar, Permeate flow=248 mL/min

### 0.2 μm Filter:—

Before diafiltration, TMP=1.1 bar, Permeate flow=284 mL/min.
After diafiltration, membrane was disinfected with 1M NaOH (40° C.) for 30 min, then 50 min, then 30 min again, and rinsed with MQ water. 0.1M NaOH was circulated in the membrane for storage.
TMP=1.1 bar, Permeate flow=228 mL/min.

### 100 kDa Filter:—

Before diafiltration, TMP=1.3 bar, Permeate flow=136 mL/min.
After diafiltration, membrane is disinfected with 1M NaOH (40° C.) for 15 min, and rinsed with MQ water. 0.1M NaOH was circulated in the membrane for storage.
TMP=1.3 bar, Permeate flow=120 mL/min

### Diafiltration

100 mL of 8.7 g/L polymer particle suspension was diafiltered with 2 L of 50 mM Potassium phosphate buffer, pH 7.5. The filtration was conducted until the last volume in 2 L was used up (approx 18-20 min) TMP was constantly maintained through variable pump feed flow rate.

### Results

### Retentate Analysis

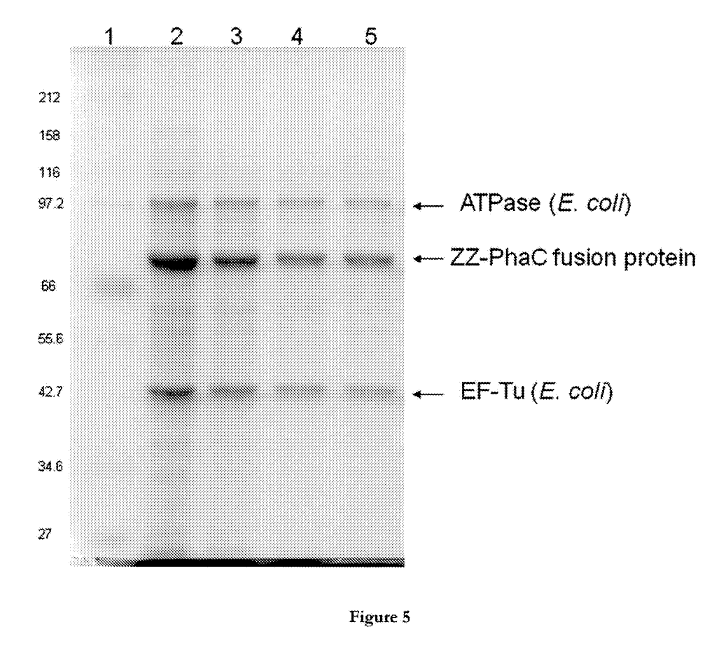

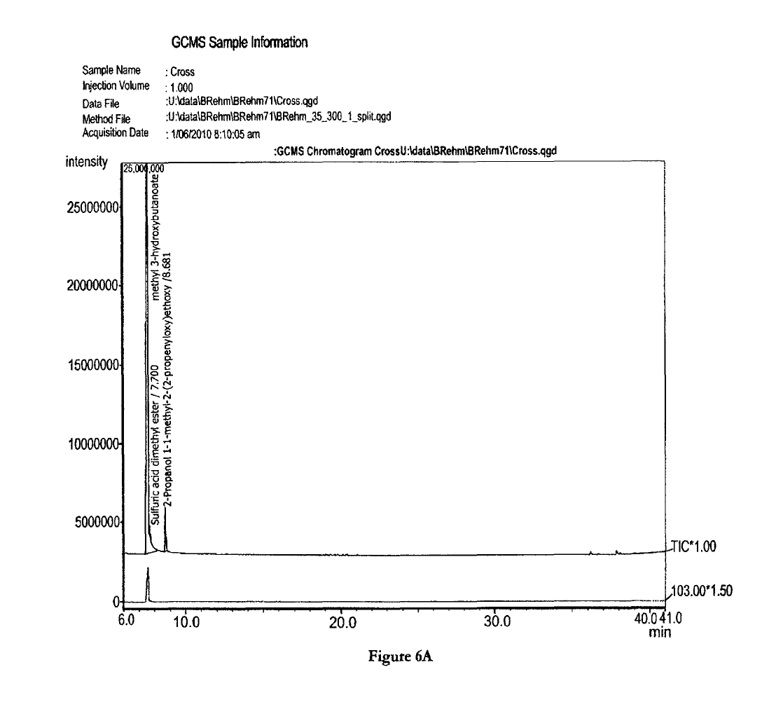

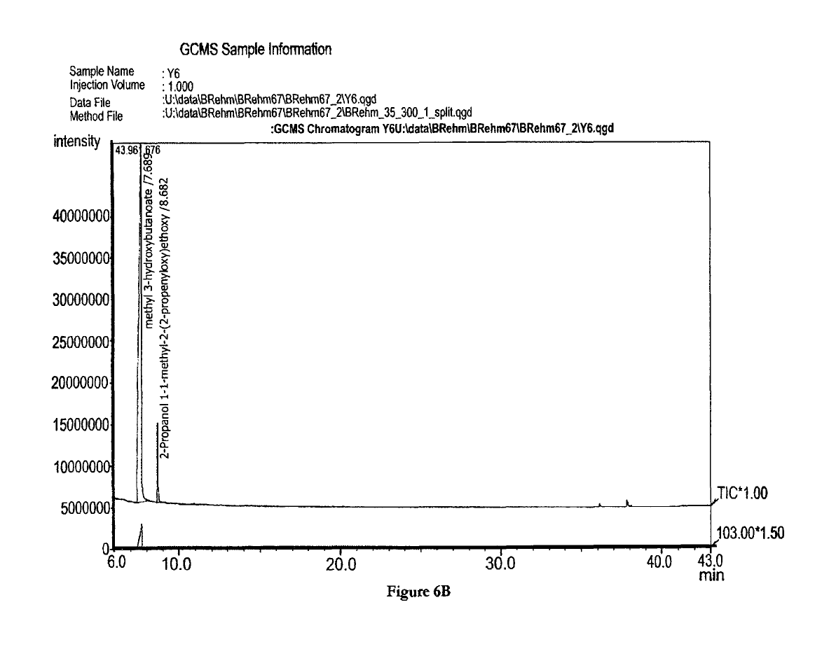

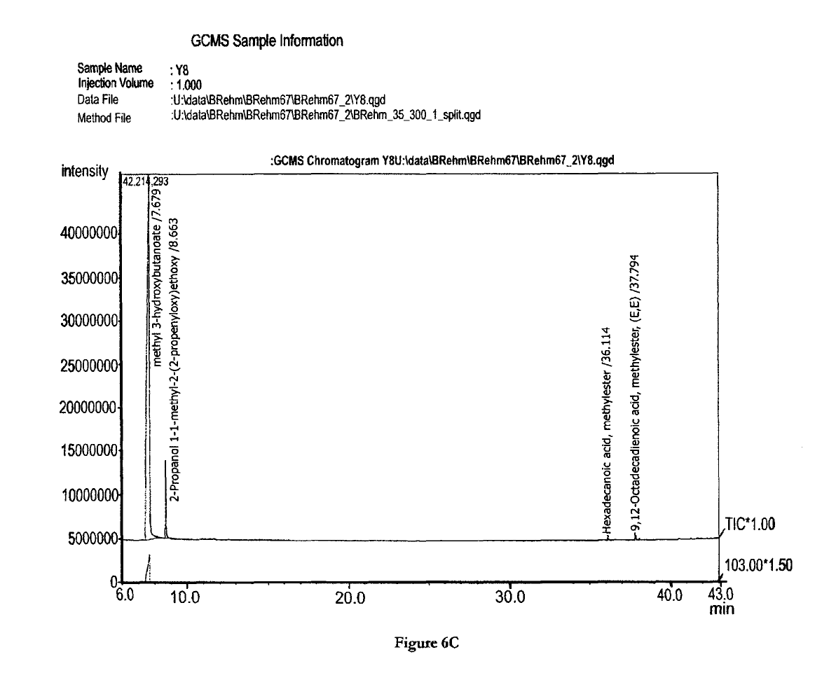

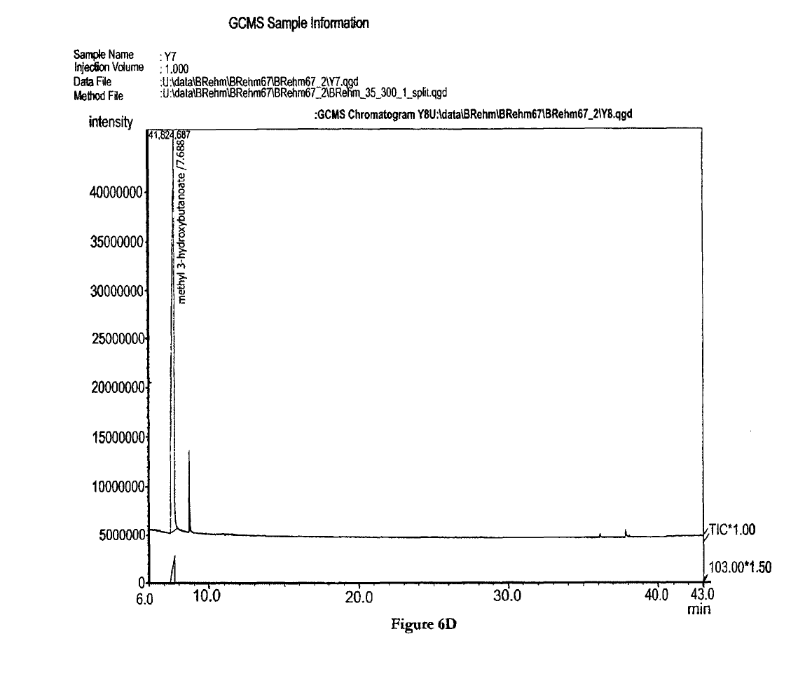

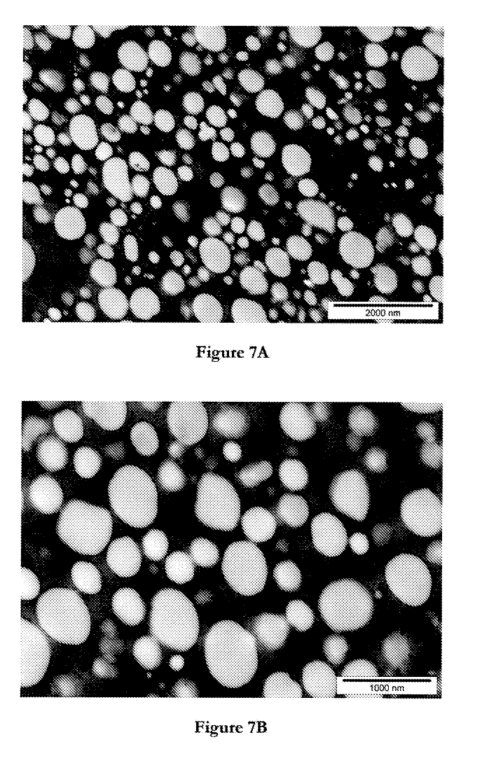

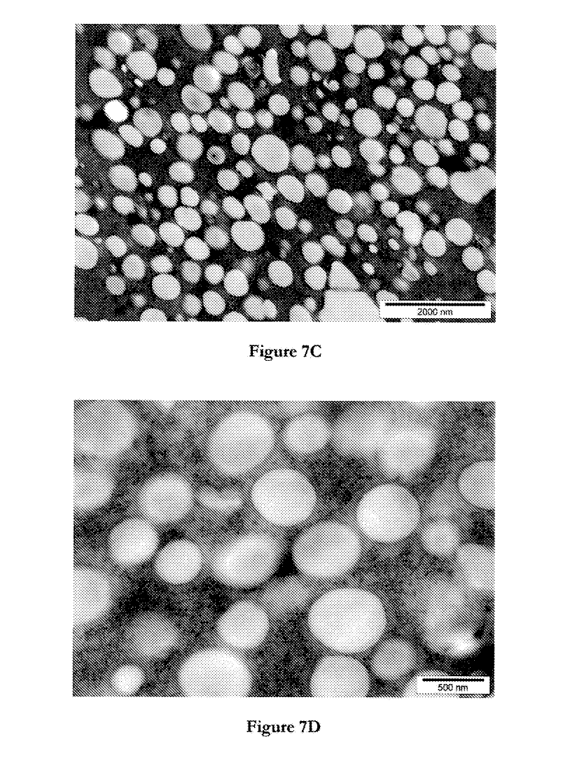

SDS-PAGE of the polymer particle protein profiles (Coomassie blue staining, see FIG. 5), IgG purification, GC/MS (see FIG. 6), and TEM (see FIG. 7) analyses were conducted on the original feedstocks (FIGS. 6A and 7A, respectively) and on the retentate of the tangential-flow filtration (FIGS. 6B and 7B, respectively).

As can be seen, excellent flow rate, permeate collection rate, and ultimately highly efficient purification of IgG immunoglobulins was achieved using the polymer particles of the present invention. GC/MS analysis showed removal of fatty acid contaminants.

These data also show that the polymer particles of the invention do not suffer deformation (see FIG. 7B) or damage (FIG. 6B and FIG. 7B) under the conditions typically used for tangential-flow filtration. GC/MS analysis showed no evidence for polymer degradation products. These data indicate that tangential-flow technologies (including membranes, filters and filter apparatuses) using polymer particles of the present invention are resistant to damage and fouling and so will not contaminate target materials, and can readily be regenerated for re-use.

### Discussion

This example clearly demonstrates that the methods of the present invention employing polymer particles as described herein in tangential-flow filtration techniques are well-suited to the separation and preparation of valuable target molecules from complex mixtures.

### Example 2

### Purification of Human IgG from a Mixed Solution

### Introduction

This example describes the use of Z-domain-comprising polymer particles and TFF in the purification of human IgG from a mixed solution.

### Materials and Methods

5 g polymer particles of the present invention comprising the Z-domain (as described in Example 1 above) were added to a solution of BSA and IgG (50 ml suspension in PBS pH 7.4). The IgG was allowed to bind to the polymer particles for a set period (30 min). After pre-incubation the polymer particle-IgG-BSA suspension was diafiltered on a TFF (0.1 um membrane) for several diafiltration volumes. The protein present in the collected permeate fractions was measured by absorbance at 280 nm (A280, 50 ml fractions), and the elution profile was plotted (to observe removal of BSA). IgG was eluted by adding citrate pH 3.0 when A280 reached zero, and IgG elution was followed in the permeate by A280 and analysis of the permeate fractions by SDS-PAGE (with silver stain).

### Results

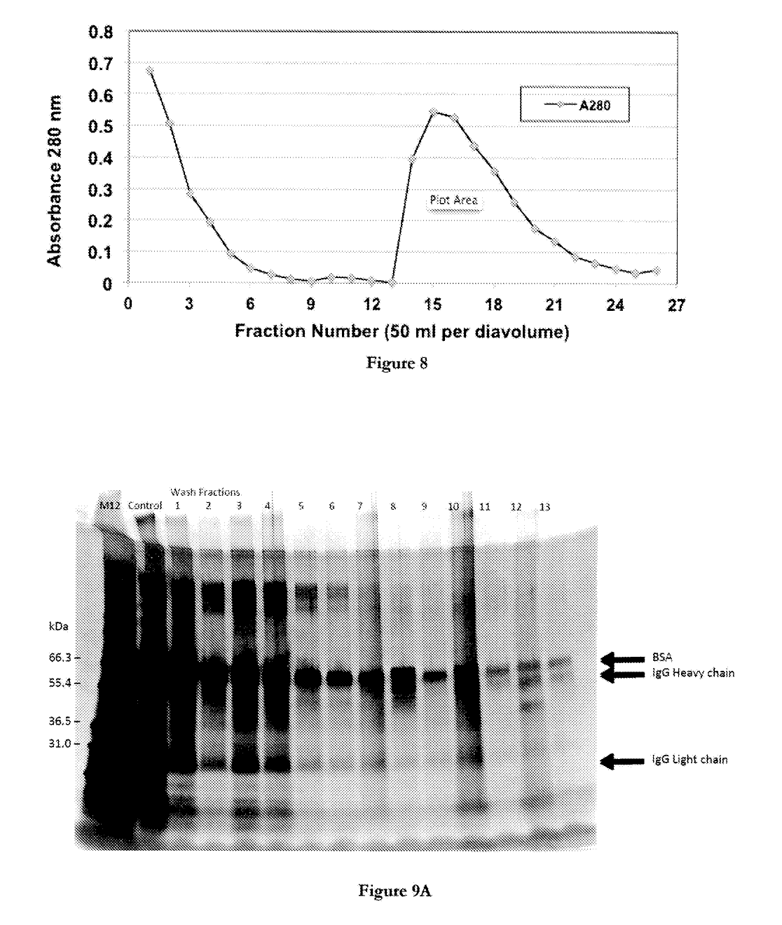

As shown in FIG. 8, BSA accounted for the initial A280 absorbance. At Fraction 13, when the absorbance in the permeate fractions was zero, citrate was added and used as the diafiltration buffer while IgG was eluted. The relative amounts of IgG and BSA were calculated using the molar extinction coefficients of each. This calculation revealed that there was quantitative recovery of IgG and BSA in the permeate fractions in the proportions they were prepared in the original solution.

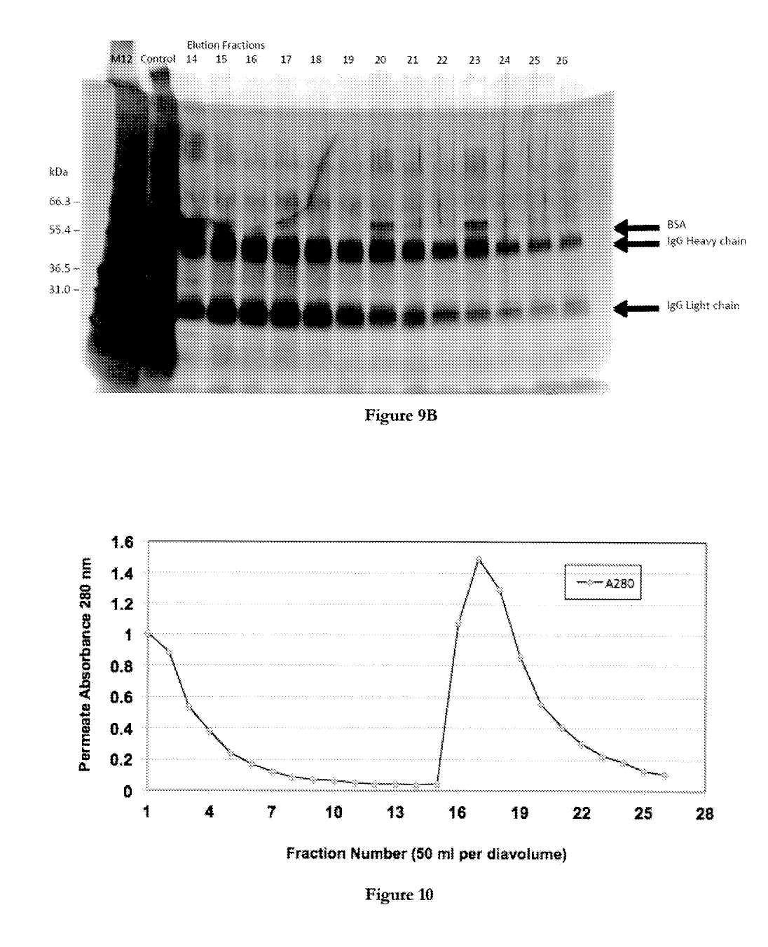

To confirm that the first peak and second peak of A280 were BSA, and IgG, respectively, SDS-PAGE was run to examine the protein in each of the permeate fractions. The gel in FIG. 9A shows the original solution and each of the permeate fractions 1-13 from the TFF analysis. The gel confirms the presence of primarily BSA in the pre-elution permeate fractions (with trace amounts of unbound IgG present). The second gel, shown in FIG. 9B, was run to observe the protein in permeate fractions 14-26. These fractions were collected immediately after the addition of citrate (pH 3.0) and the consequent elution of IgG.

These results showed that IgG was primarily eluted from the polymer particles into the permeate.

### Discussion

This result is a clear demonstration that the methods of the present invention utilising polymer particles comprising the Z-domain in TFF were effective to purify IgG from a solution comprising contaminating protein (BSA). Similarly, the methods of the invention were effective to prepare a solution from which IgG had been removed, demonstrating the utility of the methods of the present invention in the preparation of, for example, immunoglobulin-free serum.

### Example 3

### Use of GB1-Domain Polymer Particles and TFF to Purify Goat IgG from Goat Serum

### Introduction

This example describes the preparation and use in TFF of polymer particles comprising the GB1 domain of protein G to purify goat IgG from a complex mixture.

### Materials and Methods

### Construction of Expression Plasmid

The plasmid pET-14b PhaC-(GB1)3 was constructed as follows. A DNA sequence (SEQ ID NO. 1 in the attached Sequence ID Listing) encoding an N-terminal linker (LEVLAVIDKRGGGGGSGGGSGGGSGGGG, [SEQ ID NO. 2]) and three GB1 binding domains from protein G (Streptococcus sp.), each separated by a linker region (SGGGSGGGSGGGGS, [SEQ ID NO. 3]) was synthesized by Genscript Inc. The introduced XhoI/BamHI sites were used to replace the MalE encoding DNA region in plasmid pET14b PhaC-MalE (Jahns and Rehm, 2009). This resulted in plasmid pET-14b PhaC-(GB1)₃ with the DNA sequence depicted as SEQ ID NO. 4 in the attached Sequence ID Listing.

Introduction of plasmid pET-14b PhaC-(GB1)3 into E. coli strains harbouring plasmid pMCS69 (Amara and Rehm, 2003) enabled production of PHB polymer particles displaying (GB1)3.

### Purification of IgG

A TFF based IgG binding and purification protocol was used to bind and purify IgG from goat serum. A 5 mL sample of goat serum was added to 35.5 ml (5 g) polymer particles of the present invention comprising the GB1-domain suspension. The polymer particles were added at a level calculated to allow total binding of IgG (e.g. 5 g wet weight polymer particle suspension>220 mg IgG binding capacity) and adjusted to a final volume of 50 ml to create a final serum dilution of 1:10 in PBS. The mixture was diafiltered against PBS until serum proteins were fully removed as measured by A280 nm (FIG. 10) and SDS-PAGE (FIG. 11). Diafiltration was performed with a 50 cm² hollow fibre cartridge with a 0.1 nm pore size. Once the serum proteins were removed the retentate was concentrated to 20 ml and then goat IgG was eluted using NaCitrate-saline at pH 3.0 in a 50 ml retentate.

### Results

The data shown in FIGS. 10 and 11 demonstrate that IgG was successfully removed from goat serum, thus producing serum proteins free of IgG, and that purified IgG was then able to be eluted from the polymer particles in the retentate and released through the permeate with diafiltration in low pH buffer.

### Discussion

This result demonstrates that the methods of the present invention utilising polymer particles comprising the GB1-domain in TFF were effective to purify first an IgG-free serum protein preparation, and then IgG, from a complex mixture. Thus, by choosing appropriate diafiltration conditions, the methods of the present invention are able to sequentially provide desired component fractions from complex mixtures.

### Example 4

### The Use of Gold-Binding Peptide Polymer Particles and TFF to Purify Inorganic Colloidal Gold from Water

### Introduction

This example describes the use of TFF and polymer particles of the present invention comprising a gold-binding domain in the removal of an inorganic material (colloidial gold) from a solution.

### Methods

Polymer particles comprising a gold-binding peptide (see Jahns, A. C et al., Bioconjugate Chem. 2008, 19, 2072-2080) were prepared as described. A 0.005% solution of 10 nm colloidial gold particles was prepared in 30 ml of deionized water and equilibrated on a 20 cm², 0.2 um hollow fibre microfiltration cartridge. The system was run under full recirculation, feeding the permeate back into the retentate vessel. The amount of the gold particles was measured by absorbance at 520 nm. After measuring the absorbance of the colloidial gold solution in fractions of the permeate, 30 mg (final 1 mg/ml) of the polymer particles were added to the retentate reservoir. Fractions of the permeate were sampled from the feed stream at 4 minute intervals. At 8 and 29 minutes an additional 300 mg of polymer particles were added to the retentate to further bind the residual gold in solution.

### Results

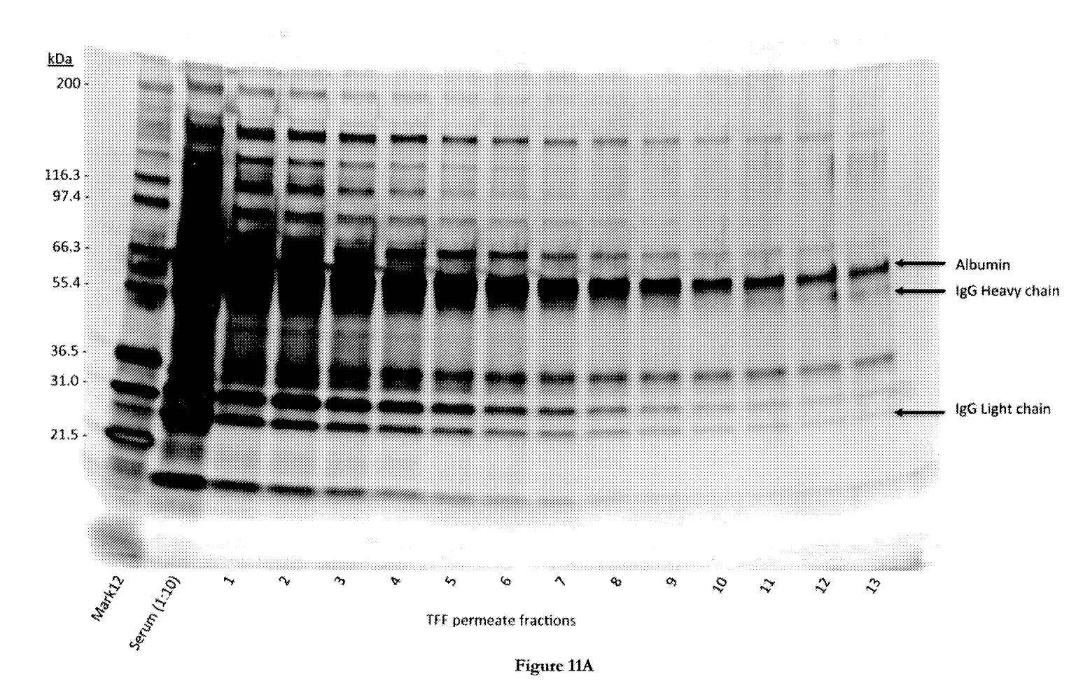

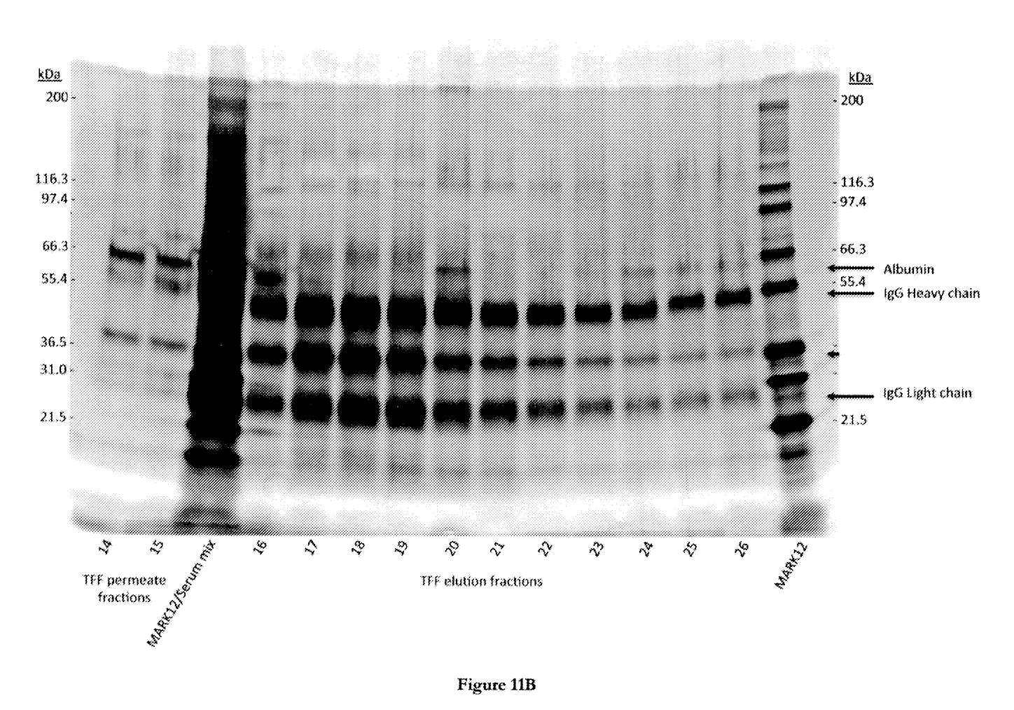

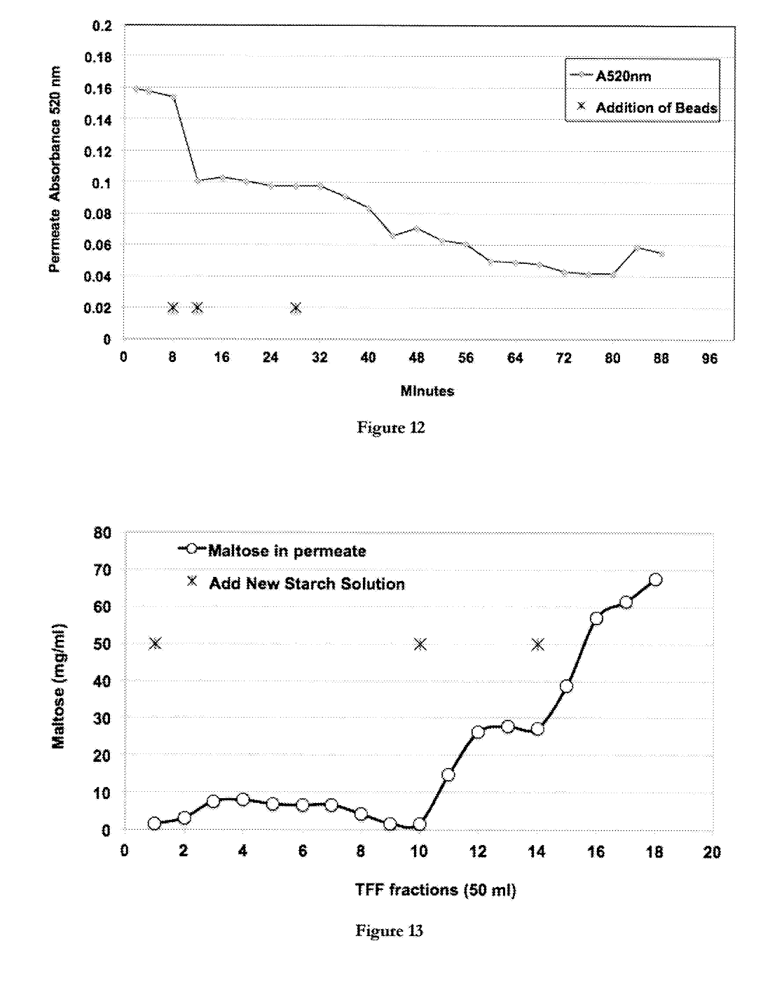

The reduction in levels of colloidial gold in the permeate stream is readily seen in FIG. 12, clearly demonstrating the efficacy of TFF and the polymer particles comprising a gold binding domain in sequestering gold particles from a solution.

### Discussion

This example clearly demonstrating the efficacy of the methods of the present invention in the recovery of inorganic compounds and their removal from a solution. Such methods have utility in both circumstances where the inorganic compound is valuable, and its recovery is desirable, and where the inorganic compound is a contaminating compound and it is desirable to remove it from the solution or other components present in the solution.

### Example 5

### The Use of Amylase-Linked Polymer Particles and TFF for the Production and Recovery of Maltose from Soluble Starch

### Introduction

This example describes the use of TFF and polymer particles comprising an enzyme in bioprocessing of biomolecules. Here, polymer particles comprising amylase were used to convert soluble starch suspensions to maltose.

### Methods

Polymer particles comprising amylase (see Rasiah, I., Rehm, B. H. A., (2009) One-step production of immobilised a-amylase in recombinant Escherichia coli. Appl Environ. Microbiol. 75:2012-2016) were prepared as described. PBS suspensions (300 ml) of soluble starch at 1% w/v, 4% w/v and 8% w/v were incubated (batch processed) with 2 g of polymer particles with shaking at 50° C. After 2 hours of incubation the 1% starch-polymer particle suspension was diafiltered into a TFF system fitted with a 110 cm², 0.1 um microfiltration cartridge. Permeate fractions were collected (50 ml) and later measured for maltose concentration. Additionally the maltose was washed from the polymer particles by diafiltering through 200 ml PBS buffer.

After the 1% solution was completely harvested, the 4% starch-polymer particle suspension was added to the same retentate and diafiltered through the system, and in the same manner maltose was recovered from the 8% suspension. Once the TFF had been completed the amount of maltose recovered in the permeate was determined (FIG. 13).

### Results

As can clearly be seen in FIG. 13, the amylase enzyme bound to the polymer particles was able to catalyse the production of maltose within the TFF system, demonstrating that the particle-linked catalyst can be used in a high temperature application and the product (maltose) can be readily recovered from the reaction suspension.

### Discussion

In this example, polymer particles and high molecular weight starch remain in the retentate fraction allowing the hydrolysis to occur while the low molecular weight product maltose can be continuously separated into permeate fraction.

This example clearly demonstrates the efficacy of the methods of the present invention in the conversion of a starting material to a desired product mediated by polymer particle-bound enzymatic activity, and the recovery of that product from a solution.

### Example 6

### Production of Catalytic Polymer Particles for Use in TFF for Decontamination and Detoxification of Water

### Introduction

This example describes the preparation of a vector for the production of polymer particles comprising the organophosphohydrolase (OpdA) from A. radiobacter and expression of the particles in Escherichia coli. The OpdA was N-terminally fused via a designed linker region to the C-terminus of polymer particle-forming enzyme PhaC of Ralstonia eutropha (see Blatchford et al., in press Biotech. Bioeng.).

### Materials and Methods

### Bacterial Strains and Growth Conditions

Bacterial strains used in this study are listed in Table 1 below. All E. coli strains were grown at 37° C. unless otherwise stated. When required, antibiotics were added at the following concentrations: ampicillin (75 μg/ml), chloramphenicol (50 μg/ml), and tetracycline (12.5 μg/ml). For polymer particle production, cells were grown at 37° C. to an OD₆₀₀ of 0.45 then induced with the addition of 1 mM IPTG. After induction, cultures were cultivated at 30° C. in shaking flasks for 44 hours.

TABLE 1
Bacterial strains, plasmids and oligonucleotides

| | | |
|---|---|---|
| Strain, | | |
| plasmid or | | |
| oligonucleotide | Genotype, description or sequence | Reference or source |

Strains

| | | |
|---|---|---|
| E. coli | recA1, endA1, gyrA96, thi-1, hsdR17 | Stratagene |
| XL1-Blue | (r−ₖ, m+ₖ), supE44, relA1, lac [F′, | |
| | proAB, lacIq, lacZΔM15, Tn10(Tc^{r})] | |
| E. coli | F−; ompT; hsdS_{B}(r_{B}− m_{B}−); gal; dcm | Novagen |
| BL21 λ(DE3) | λ(DE3) | |

Plasmids

| | | |
|---|---|---|
| pET14b | Ap^{r}; T7 Promoter | Novagen |
| pETC | pET14b derivative coding for the phaC | Peters, V., and B. H. |
| | wild type under T7 promoter control | Rehm. (2008). J. |
| | | Biotechnol. 134:266-74. |
| pMCS69 | pBBR1MCS derivative containing | Amara, A., Rehm, B. H.A |
| | genes phaA and phaB of R. eutropha | (2003). Biochem. J. |
| | colinear to lac promoter; Cm^{r} | 374:413-421. |
| pETMCSI | T7 expression vector based on pET3c. | Jackson, C. J., et al., |
| | Ap^{r} | (2006). Biochem. J. |
| | | 397:501-508. |
| pET14b PhaC- | pET14b derivative containing malE | Jahns A.C., Rehm B.H.A. |
| linker-MalE | fused to the 3′ end of phaC via a | (2009). Appl Environ |
| | linker sequence | Microbiol. 75:5461-6. |
| pET14b PhaC- | pET14b derivative containing opdA | This example |
| linker-OpdA | fused to the 3′ end of phaC via a | |
| | linker sequence | |
| pGEM-T Easy | Ap^{r}; P_{lac} | Invitrogen |

Oligonucleotides

| | | |
|---|---|---|
| 5′ - XhoI | 5′-GGA CTC TCG AGA GCA TGG | Sigma Aldrich |
| | CCC GAC CAA TCG GTA CAG-3′ | |
| | [SEQ ID NO. 5] | |
| 3′ - BamHI | 5′-GTA CAG GAT CCT CAC GAC | Sigma Aldrich |
| | GCC CGC ACG GTC GGT GAC | |
| | AAG-3′ [SEQ ID NO. 6] | |

Tc^{r} - tetracycline resistance,
Ap^{r} - ampicillin resistance,
Cm^{r} - chloramphenicol resistance

### Plasmids and Oligonucleotides

Plasmids used in this study are listed in Table 1 above. General cloning procedures and DNA isolation were performed using methods generally known in the art. DNA primers, deoxynucleoside triphosphate, T4 DNA ligase and Tag polymerase were purchased from Integrated DNA Technologies, (CA, USA). Chemical reagents were purchased from Sigma Aldrich (St. Louis, Mo.).

### Construction of Plasmas for Production of Functional OpdA Polyester Granules

The opdA DNA sequence encoding the organophosphate degradation protein was obtained from the CSIRO, Can berra, Australia as an insert within the plasmid pETMCSI Primers were designed with engineered 5′ and 3′ restriction sites. The 5′ primer (5′-XhoI, [SEQ ID NO. 5] harbors an XhoI restriction site and the 3′ primer (3′-BamHI, [SEQ ID NO. 6] harbors a BamHI restriction site. A Pfx PCR was performed with the Sigma manufactured primers to amplify the OpdA encoding region. The fragment was poly A-tailed and ligated into the plasmid pGEM-T Easy. Transformants were screened using blue/white selection on indicator plates. Recombinant white colonies were screened for the opdA insert by sequencing. The opdA sequence was cleaved from the plasmid pGEM-T Easy by hydrolysis with the XhoI and BamHI restriction enzymes. The plasmid pET14b PhaC-linker-MalE was used as a suitable vector as the inclusion of a linker region was deemed necessary after hydrophobicity analysis of the N-terminus of the OpdA protein. The plasmid pET14b PhaC-linker-MalE was hydrolysed with XhoI and BamHI to cleave the MalE region from the plasmid. The opdA sequence was cloned into the XhoI and BamHI sites of the plasmid backbone pET14b PhaC-linker resulting in the plasmid pET14b PhaC-linker-OpdA. This was used to transform E. coli BL21 λ(DE3) competent cells harboring the plasmid pMCS69, which mediates the synthesis of the precursor R-3-hydroxybutyryl-coenzyme A (CoA) required for polymer particle formation. The DNA sequence of the new plasmid construct was confirmed by DNA sequencing. To produce control polymer particles that do not display OpdA activity, the plasmid pETC, which encodes only the wild-type PhaC of R. eutropha, was used in E. coli BL21 λ(DE3) in the presence of plasmid pMCS69.

### Protein Analysis

Polyester protein profiles were analyzed by sodium dodecyl sulfate (SDS)-polyacrylamide gel electrophoresis (PAGE) as described in Laemmli, U. K. 1970. Cleavage of structural proteins during the assembly of the head of bacteriophage T4. Nature 227:680-5. Gels were stained with Coomassie brilliant blue G250. Protein concentrations were determined using the Bradford protein quantification method.

### Tryptic Peptide Fingerprinting Analysis Using MALDI-TOF Mass Spectrometry

In order to identify the PhaC-OpdA fusion protein, the protein band of interest was cut out of the gel and subjected trypsin digest followed by MALDI-TOF mass spectrometry of the resulting tryptic peptides as previously described (15).

### Polyester Analysis

Production of the polyester, polyhydroxybutyrate, which indicated in vivo activity of the polyester synthase, was determined qualitatively and quantitatively by gas chromatography/mass spectrometry (GC/MS) as described in Brandl, H., R. A. Gross, R. W. Lenz, and R. C. Fuller. 1988. Pseudomonas oleovorans as a Source of Poly(beta-Hydroxyalkanoates) for Potential Applications as Biodegradable Polyesters. Appl. Env. Microbiol. 54:1977-1982.

### Polymer Particles

Polymer particles were isolated from recombinant E. coli cells using mechanical cell disruption and ultracentrifugation on a glycerol gradient as described in Jahns, A. C., R. G. Haverkamp, and B. H. Rehm. 2008. Multifunctional inorganic-binding polymer particles self-assembled inside engineered bacteria. Bioconj. Chem. 19:2072-80. Control polymer particles were produced from E. coli BL21 λ(DE3) cells harboring pMCS69 and pETC.

### Microscopy

Polyester granules produced inside bacterial cells were visualized with fluorescence microscopy following Nile red staining as described in Peters, V., and B. H. Rehm. 2005. In vivo monitoring of PHA granule formation using GFP-labeled PHA synthases. FEMS Microbiol. Lett. 248:93-100.

### Enzyme Assays

The phosphotriesterase activity of polymer particle bound PhaC-OpdA and polyester bound PhaC were determined using methods described by Dumas and coworkers (Dumas, D. P., S. R. Caldwell, J. R. Wild, and F. M. Raushel. 1989 Purification and properties of the phosphotriesterase from Pseudomonas diminuta. J. Biol. Chem. 264:19659-19665) and Harcourt et al. (Harcourt, R. L., I. Home, T. D. Sutherland, B. D. Hammock, R. J. Russell, and J. G. Oakeshott. 2002. Development of a simple and sensitive fluorimetric method for isolation of coumaphos-hydrolysing bacteria. Lett. Appl. Microbiol. 34:263-268) using methyl parathion (O,O-diethyl O-(4-nitrophenyl) phosphorothioate; Sigma) as substrate.

The rate of hydrolysis of methyl parathion was monitored by the increase in absorbance at 405 nm, caused by the liberation of para-nitrophenol, using a Spectromax 190 spectrophotometer (Molecular Devices). Units of enzyme activity are defined as μmol methyl parathion turned over per minute.

### Results

The plasmid pET14b PhaC-linker-OpdA encoding the full length OpdA translationally fused to the C terminus of the polyester synthase PhaC mediated formation of polyhydroxybutyrate (PHB) polymer particles in recombinant E. coli B121 λ(DE3), resulting in an overall polyester content over biomass of about 48% which was slightly less when compared to a content of 59% produced by recombinant E. coli B121 λ(DE3) expressing only wild type polyester synthase. The formation of spherical particles inside the cells was additionally confirmed by fluorescence microscopy of Nile red stained cells (data not shown). Analysis of proteins attached to isolated polymer particles clearly showed overproduction of the PhaC-OpdA fusion protein, the identity of which was confirmed by tryptic peptide fingerprinting analysis (data not shown).

Polymer particles displaying PhaC and the PhaC-OpdA fusion were tested for phosphotriesterase activity using methyl parathion as a substrate. The PhaC polymer particles had no detectable phosphotriesterase activity, whilst the PhaC-OpdA fusion protein displaying polymer particles had approximately 1,840 U of activity per gram of wet polymer particle mass.

### Discussion

This example shows that catalytic polymer particles of the invention can be produced wherein the enzyme, in this case OpdA, is efficiently immobilized at high density and functionality, by fusion to the C terminus of the polyester synthase, PhaC. These polymer particles can be produced at industrial scale using standard bacterial fermentation techniques, and are suitable for use in TFF.

### Example 7

### The Use of Catalytic Polymer Particles for TFF-Based Conversion and Detoxification

In this example, an organic low molecular weight compound was catalyzed and separated from a solution via the use of polymer particles comprising enzyme moieties using TFF. Additionally it was demonstrated that the enzyme activity could be recycled in this process.

### Methods

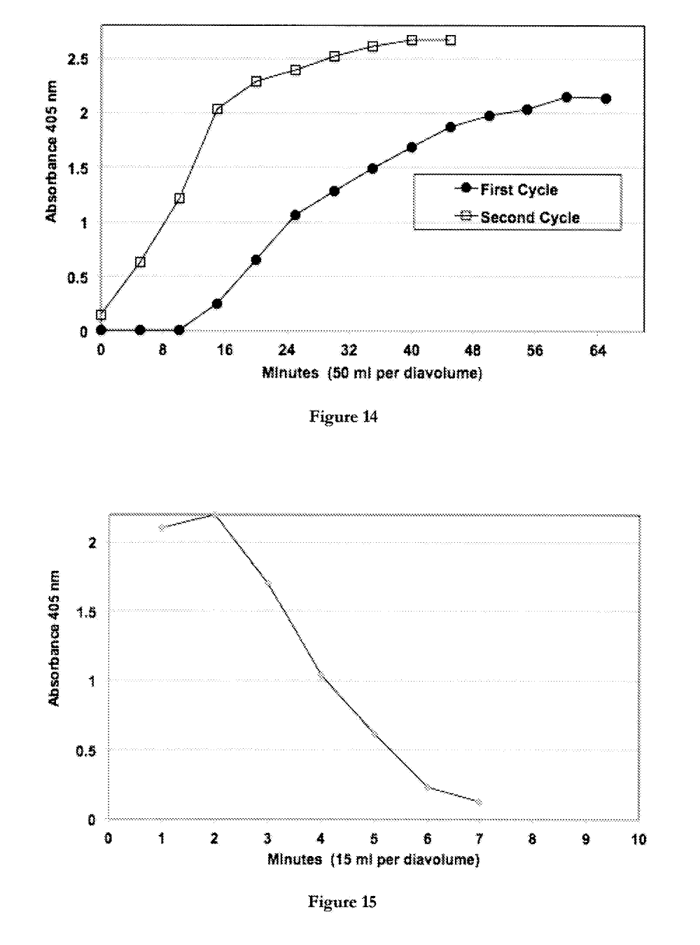

A 50 ml solution of 200 μM Methyl parathion in CHES buffer formed the retentate solution. The TFF system using a 0.2 μm 20 cm² PES microfiltration cartridge was employed and the 50 ml TFF retentate was run under full recirculation with 0.5 ml fractions collected every 5 minutes (˜30 ml or 1 DV), measured for absorbance and returned to the retentate. After running for 10 minutes, 500 mg of polymer particles were added to the suspension and the conversion of MP to PNP was observed at 405 nm (FIG. 14). After the reaction was completed the retentate was diafiltered to remove all of the PNP with 50 ml fractions collected (FIG. 15). After all of the PNP was removed the remaining polymer particles were resuspended to 45 ml and 5 ml MP concentrate was added to create the 200 μM Methyl parathion concentration and the batch conversion process was repeated under full re-circulation and the reaction progress was measured at 5 minute intervals at 520 nm (FIG. 14).

### Results

As can clearly be seen in FIGS. 14 and 15, the methods of the present invention can be utilised to remove, via multiple cycles of catalytic processing, contaminating low molecular weight organic compounds. FIG. 14 clearly shows the rapid conversion of methy parathion to para-nitrophenol on contact with the polymer particles during TFF. Following from FIG. 14, after completion of the catalytic conversion of methyl parathion to paranitrophenol, the 30 ml suspension of polymer particles was diafiltered with CHES buffer. The removal of paranitrophenol was monitored by measuring fractions of permeate for absorbance at 405 nm. Once the paranitrophenol was removed the polymer particles could be recycled for further rounds of detoxification.

### Example 8

### Use of Tangential Flow Filtration in the PHB Polymer Particle Purification

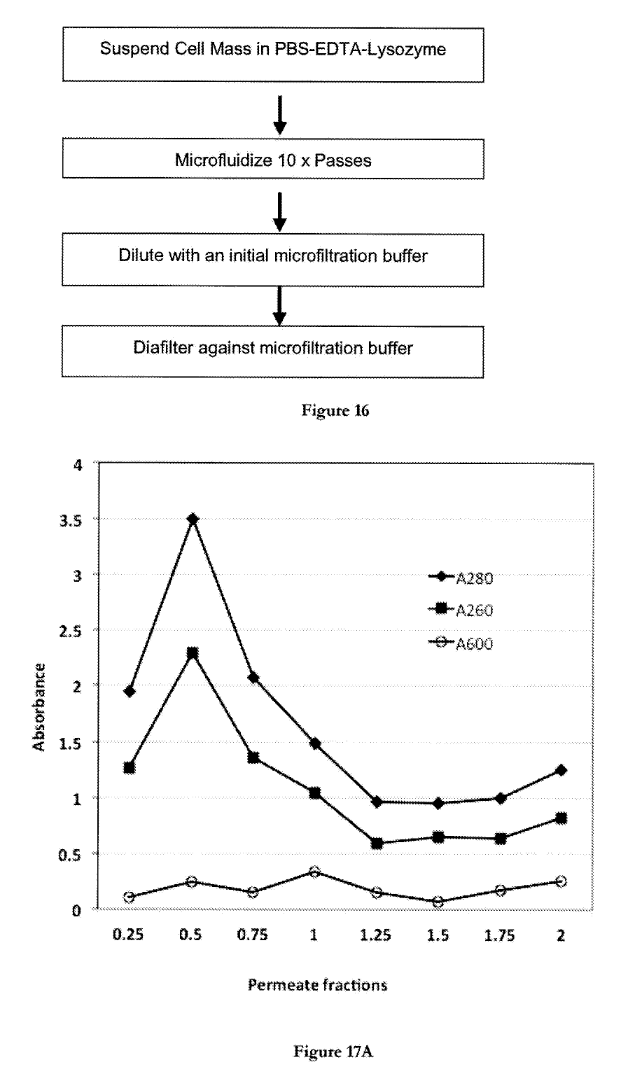

This example describes the generalised use of TFF for purification of PHB polymer particles in a range of scales and for a range of applications. In one exemplary embodiment, TFF is used to remove host cell contaminants from a cell homogenate. Bacterial biomass is suspended in a buffer solution including process excipients such as lysozyme and EDTA (to destabilize the bacterial cell wall) and is lysed by any of a range of methods well known in the art, including sonication, french pressure cell or microfluidization. Once lysed, small subcellular components are separated from the polymer particles using microfiltration at a desired pore size (e.g. 0.1 μm-0.45 μm). A representative process scheme is shown in FIG. 16.

### Example 9

### Removal of Host Cell Contaminants by TFF Using a Hollow Fiber Crossflow Filter—Permeate Analysis

The representative process scheme in Example 9 was employed in this preparative example, which describes the preparation of a polymer particle composition using TFF.

### Methods

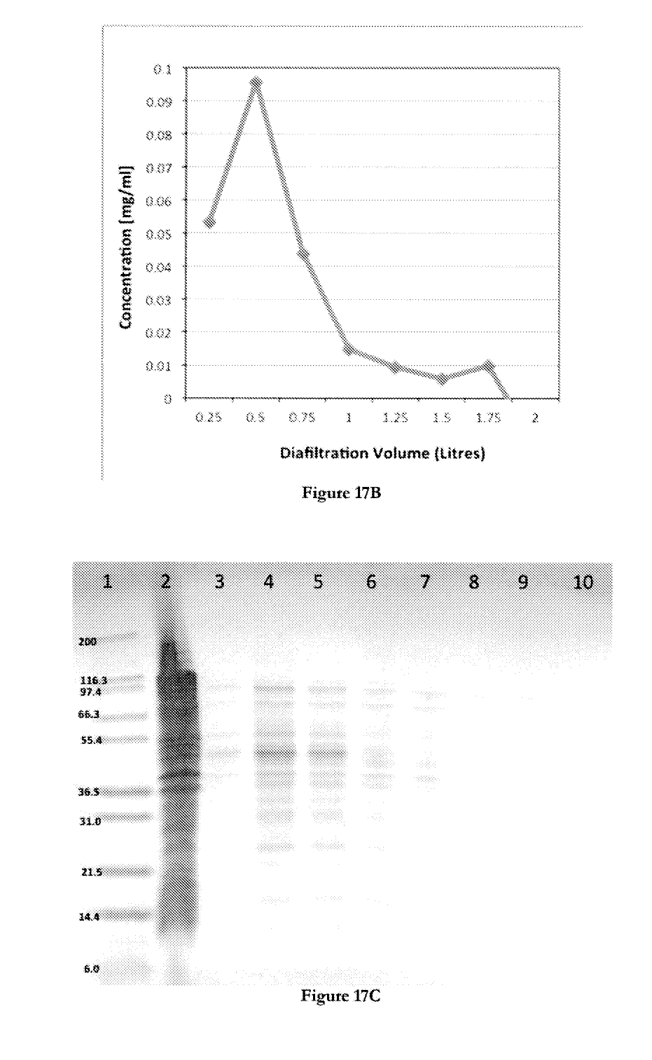

Approximately 20 g of E. coli biomass containing PHB polymer particles was microfluidized in 250 ml of Phosphate buffered saline (PBS) with 1 mM EDTA. The 250 ml homogenate was added directly to a Hollow Fiber TFF system with a 110 cm² TFF cartridge with 0.1 μm pores, and was diafiltered using 8 volumes (8×250 ml) of PBS in 20% ethanol (FIG. 17).

### Results

As can be seen in FIG. 17, the removal of host cell contaminants was readily achieved, as demonstrated by observing the content of permeate fractions by their absorbance at 260, 280 and 600 nm (FIG. 17A), the protein concentration of the permeate fractions (FIG. 17B), and by analysis of the permeate fractions on SDS PAGE (FIG. 17C). Purification was thus demonstrated as the removal of soluble protein (A₂₈₀) and nucleic acid (A₂₆₀) from the polymer particles into the permeate.

### Example 10

### The Use of a Detergent (Deoxycholic Acid) in the Purification of PHB Polymer Particles Using Hollow Fiber Tangential Flow Filtration

This example demonstrates that the dissociation of host cell proteins and membranes was further enhanced by the use of a detergent, in this example, Deoxycholic acid (DOC).

### Methods

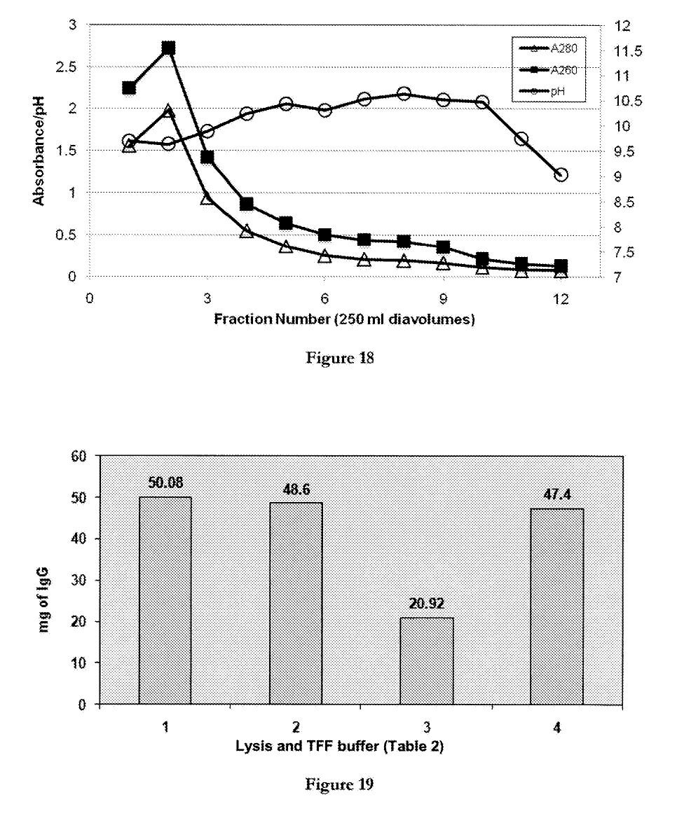

A range of buffers were employed to homogenize the host cells (so as to release PHB polymer particles), each containing 0.2% DOC. The composition of the homogenizing buffer used is outlined in Table 2 below. These same buffers were also used for diafiltration in the hollow fibre TFF process to purify the polymer particle suspension (FIG. 18). E. coli biomass (˜20 g) was microfluidized in 250 ml of the various buffers. The homogenate was later diafiltered on a 110 cm² hollow fiber tangential-flow cartridge (GE Exampler CFP-1-E-3MA) with 8 volumes (8×250 ml) of the same buffer. After additional TFF diafiltration against PBS in 20% ethanol, the polymer particles were assessed for IgG binding activity.

### Results

As can be seen in FIG. 19, under a range of increasingly harsh conditions IgG-binding activity is preserved after treatment by TFF diafiltration.

TABLE 2
Homogenization and TFF Buffer Conditions for IgG Purification.

| Buffer Type | Microfluidization/TFF Purification Condition |
|---|---|
| 1 | 50 mM Tris-EDTA pH 11 |
| 2 | 50 mM Tris-EDTA- 0.2% Deoxycholate, pH 11 |
| 3 | 50 mM Tris-EDTA-0.2% Deoxycholate, 300 mM NaCl, |
| | pH 11 |
| 4 | 50 mM Tris-EDTA + 0.1% Deoxycholate, pH 10 then |
| | TFF to 50 mM Na Citrate, saline, pH 3.0 to PBS pH 7.4. |

### Example 11

### The Use of a Detergent (Lubrol) in the Purification of PHB Polymer Particles Using Open Channel Tangential Flow Filtration

The use of detergent enhanced TFF purification of PHB polymer particles was also developed at larger scale using an open-channel crossflow system designed for pilot scale purification. Detergents such as Deoxycholate, Lubrol and SDS were demonstrated to be effective in enhancing the purification of these polymer particles.

### Methods

325 g of E. coli biomass containing polymer particles of the present invention comprising the Z-domain was homogenized by microfluidization in 0.5% Lubrol 50 mM Tris-10 mM EDTA buffer pH10. Crude polymer particles were recovered by centrifuging the cell homogenate at 16,000×g for 30 minutes to remove a supernatant laden with host cell DNA, protein and lipid.

The crude polymer particle homogenate was applied to a Millipore Prostak—4 stak membrane cartridge with a total open channel surface area of 0.34 m2. The crude polymer particle suspension was treated to diafiltration against 8 volumes of 0.1% Lubrol. 20 mM Tris-10 mM Sodium EDTA, pH 10, 8 volumes of 25 mM Sodium citrate-saline, pH 3.0 and 8 volumes of PBS in 20% Ethanol, pH 7.4. Treatment with pH 3.0 citrate buffer was designed to mimic polymer particle elution conditions and remove any residual host cell proteins which could be eluted under acidic conditions. PBS-20% Ethanol was used to control pH and salinity of the final product in an environment that does not support microbial growth.

### Results

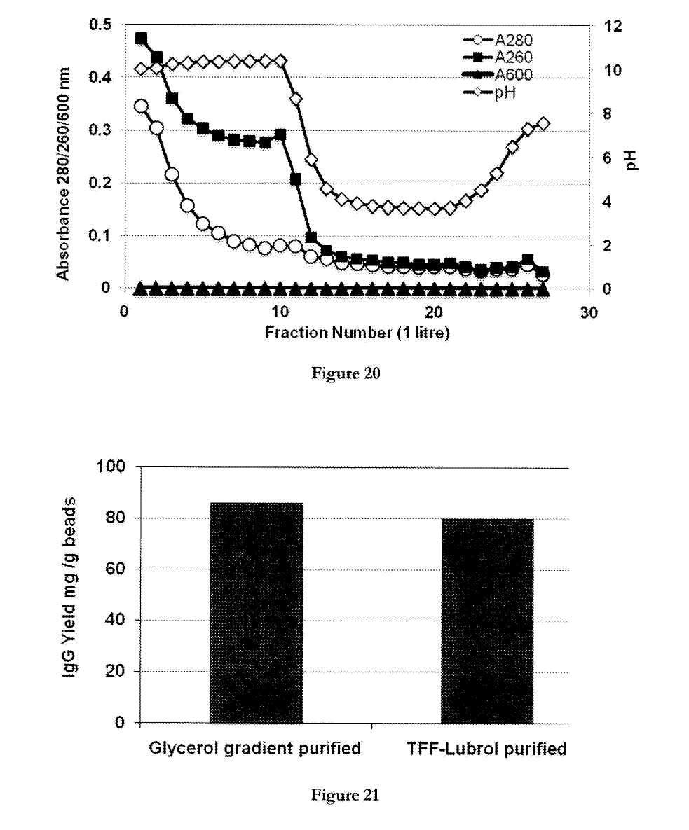

The permeate analysis shown in FIG. 20 showed substantially reduced protein and nucleic acid due in large part to the removal of these contaminants via centrifugation of the crude polymer particles prior to TFF.

The polymer particles of the present invention comprising the GB1-domain recovered from the retentate were analyzed for IgG binding activity and were compared with the binding activity of a glycerol gradient purified sample of the original host cell biomass (see FIG. 21). The binding data revealed that the resulting IgG yield derived from these polymer particles was similar for the both the non-scalable glycerol gradient process and the scalable TFF-detergent extraction process. Thus, not only was PHB polymer particle functionality preserved, but the TFF-based method used for particle preparation is amenable to large scale production.

### Example 12

### Chemical Methods to Enhance TFF Purification of Polymer Particles

This example demonstrates that the enhancement of polymer particle purification is achieved through the use of chemical extraction agents. In this procedure the bacterial homogenate is treated with the any one of a range of chemical conditions to enhance dispersion of cellular debris and purification by removal of contaminants. The range of chemical conditions can be organized into a chemical extraction matrix. In addition, the chemical treatments were tested to determine if the chemical treatment affects the activity of the polymer particles.

### Methods

A range of chemical conditions used to enhance the extraction of contaminants from PHB-polymer particles in the methods of the present invention is outlined in Table 3 below.

TABLE 3
Chemical Extraction Matrix

| Chaotropes | Acid/Base | Chelators | Detergents | Salts | Other |
|---|---|---|---|---|---|
| Urea | NaOH | EDTA | ASB | NaCl | Lipase |
| Guanidine HCl | Tris pH 8-10 | | Sarcosine | Imidazole | Thioglycerol |
| | Citrate pH 3-4 | | Tween | | |
| | Triethylamine | | Deoxycholate | | |
| | | | Chaps | | |
| | | | SDS | | |
| | | | Zwittergent | | |
| | | | Lubrol | | |

### Batch Wash Studies

Crude biomass homogenates containing polymer particles of the present invention comprising the Z-domain (an IgG binding PHB polymer particle) were extracted with a range of chemical treatments including acid and base treatments, high salt, chelators, detergents and chaotropes. The degree of contaminant removal was quantified by measuring absorbance in the supernatant after polymer particles/homogenate were centrifuged (15,000×g, 20 min). The crude polymer particle pellet was resuspended in PBS pH 7.4 and assayed for the degree of residual IgG binding (compared to a PBS control).

### Results

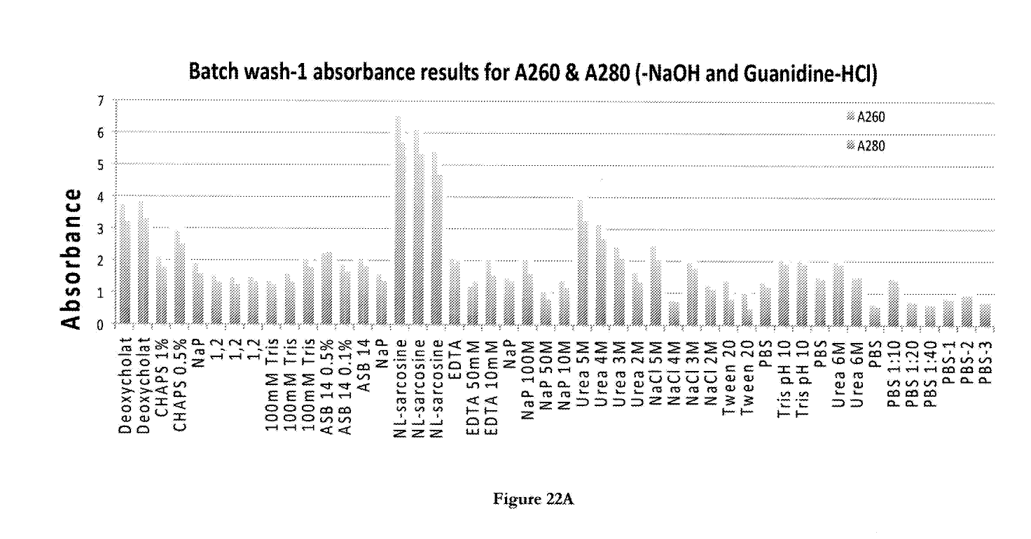

As can be seen in FIG. 22A, the degree of contaminant removal as quantified by A260 and A280 varied substantially depending on the chemical treatment employed. Furthermore, as shown in FIG. 22B, the degree of residual IgG binding varied substantially depending on the chemical treatment used in the preparative method.

### Example 13

### A Flexible Scale Process for Purification of PHB Polymer Particles

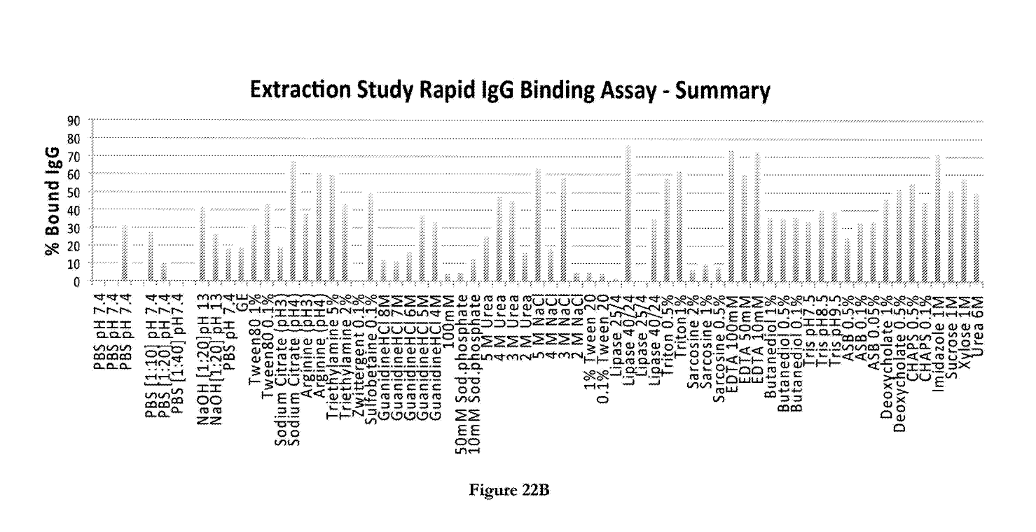

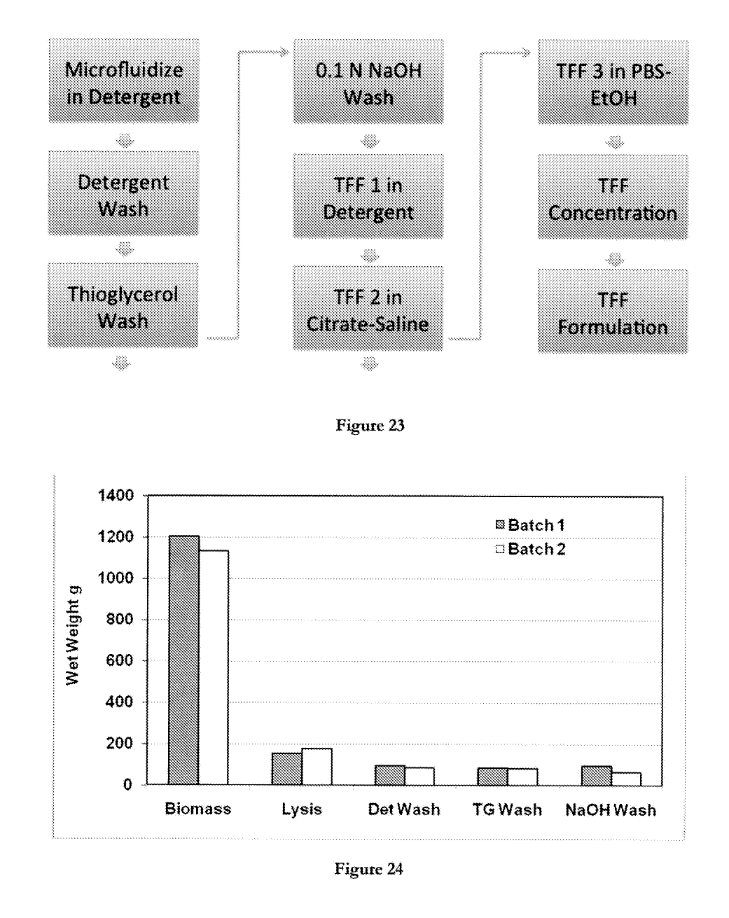

A scalable process for the purification of polymer particles incorporating both chemical extraction and TFF is illustrated in FIG. 23. This process can be run from biomass quantities of 20 g to the multi-kilogram scale. In addition the chemical extraction conditions are not limited to those specifically exemplified herein—both the detergent type, concentration and chemical treatments can be modified to suit the particular requirements of the polymer particle preparation process.

After chemical extraction the purified polymer particle mass is applied to the Prostak system loaded with scalable membrane surface from 0.17 m² to several m², depending on requirements.

Following TFF diafiltration, the purified polymer particles are stored in PBS-ethanol as recovered from the Prostak system, or are concentrated to a specific “slurry” concentration on a smaller scale hollow fiber system. A range of analytical measurements are performed to characterize the final polymer particle preparation or throughout the process, as required.

### Example 14

### Pilot Scale Extraction of PHB Polymer Particles from E. ColiBiomass

In the present example pilot scale quantities of E. coli biomass containing PHB polymer particles (1100-1200 g) were processed using the flexible scale process described in Example 13 above.

### Methods

The polymer particle were microfluidized in an SDS-Tris-EDTA alkaline solution to homogenize cells and recover crude polymer particles. In the second phase the crude polymer particle mass—which was extensively de-bulked (see FIG. 24) in the lysis process was sequentially washed with the lysis detergent, thioglycerol and 0.1 N NaOH. Between each step the polymer particles were harvested at 16,000×g for 30 minutes.

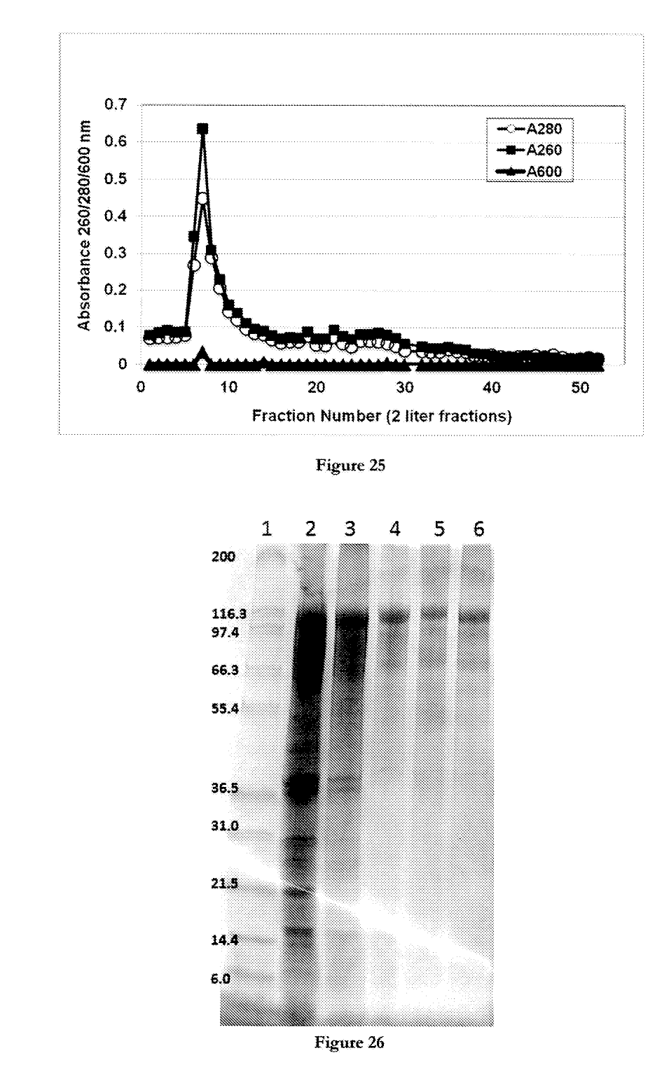

After extraction the crude polymer particles were neutralized and loaded onto the Prostak system loaded with scalable membrane surface of 0.41 m² membrane area, and diafiltered with 20 volumes of 0.04% SDS-TRIS EDTA, 7 volumes of citrate saline buffer pH 3.0 and up to 10 volumes of 10 mM sodium phosphate, 150 mM NaCl, pH 7.4 in 20% ethanol until the pH of the suspension was brought to pH 7.4 (FIG. 25).

### Results

The effectiveness of the purification process employed in this example is clearly shown by both SDS-PAGE analysis (see FIG. 26) and the assessment of endotoxin reduction in the polymer particle suspension, as shown in Table 3 below.

TABLE 3
Endotoxin levels before and after TFF purification

| Sample | Endotoxin units |
|---|---|
| DS 104-1 Pre-TFF polymer particles | <1000 EU/mg |
| | >125 EU/mg |
| Final polymer particles (End TFF 3) | <250 EU/mg |
| | >125 EU/mg |

### Discussion

This example demonstrates that the pilot scale preparation of polymer particle via TFF was readily achieved through the use of the methods described herein. Substantial removal of contaminating proteins was achieved via TFF separation from crude biomass, along with a substantial reduction in endotoxin levels. Thus, the methods of the invention are amenable to the scalable production of purified polymer particles utilising TFF methodologies.

### INDUSTRIAL APPLICATION

The polymer particles and methods of the invention have application in a wide range of purification and preparation technologies, including the separation of target substances from complex compositions and the preparation of reaction products from compositions comprising one or more reaction substrates.

1.-52. (canceled)

53. A method for preparing one or more target substances from a source material, the method comprising contacting the source material with a population of amorphous polymer particles for a time sufficient to allow the amorphous polymer particles to bind one or more target substances or one or more precursors of a target substance or one or more contaminants, separating by tangential flow filtration the one or more contaminants from the particle-bound target substance or precursor thereof or the one or more target substances or precursor thereof from a particle-bound contaminant, and recovering the target substance.

54. The method of claim 53, wherein the population of amorphous polymer particles is a heterogeneous population.

55. The method of claim 53, wherein one or more of the amorphous polymer particles comprises one or more biopolymers selected from a polyester, a polythioester or a polyhydroxyalkanoate.

56. The method of claim 55, wherein one or more of the polymer particles comprises a biopolymer selected from a polyester, a polythioester or a polyhydroxyalkanoate and
i. a polymer particle-forming polypeptide, or
ii. a polymer particle-binding polypeptide;
iii. a polypeptide fusion partner;
iv. an affinity ligand;
v. an enzyme;
vi. a fusion polypeptide comprising two or more of the above; or
vii. any combination of any two or more of (i) to (vi) above.

57. The method of claim 53, wherein one or more of the amorphous polymer particles is, or is capable of being, synthesised by a particle-forming protein.

58. The method of claim 53, wherein the one or more polymer particles comprise a ligand capable of binding the target substance.

59. The method of claim 53, wherein the target substance is one or more antibodies.

60. The method of claim 53, wherein the target substance is one or more polymer particles.

61. The method of claim 53, wherein the recovery of the target substance is by elution from the polymer particle.

62. The method of claim 53, wherein the recovery of the target substance is by collection of the tangential flow filtration permeate.

63. The method of claim 53, wherein the recovery of the target substance is by collection of the tangential flow filtration retentate.

64. The method of claim 53, wherein the target substance is one or more reaction products, the method comprising contacting by tangential flow filtration a source material comprising one or more reaction substrates with one or more polymer particles for a sufficient time to allow the one or more polymer particles to bind a desired fraction of the one or more reaction substrates, optionally separating one or more contaminants from the polymer particles by tangential flow filtration, and recovering the reaction product, wherein the one or more polymer particles comprise a catalyst of the reaction, and wherein one or more of the polymer particles comprises
i. a biopolymer selected from a polyester, a polythioester or a polyhydroxyalkanoate; or
ii. a polymer particle-forming polypeptide; or
iii. a polymer particle-binding polypeptide;
iv. a polypeptide fusion partner;
v. an affinity ligand;
vi. an enzyme;
vii. a fusion polypeptide comprising two or more of the above; or
viii. any combination of any two or more of (i) to (vii) above.

65. A method for separating or purifying one or more amorphous polymer particles from a source material, the method comprising separating one or more contaminants from the amorphous polymer particles by tangential flow filtration, and recovering the one or more amorphous polymer particles, wherein one or more of the amorphous polymer particles comprises
i. a biopolymer selected from a polyester, a polythioester or a polyhydroxyalkanoate; or
ii. a polymer particle-forming polypeptide, such as a polymer synthase or a polymer synthase fusion; or
iii. a polymer particle-binding polypeptide;
iv. a polypeptide fusion partner;
v. an affinity ligand;
vi. an enzyme;
vii. a fusion polypeptide comprising two or more of the above; or
viii. any combination of any two or more of (i) to (vii) above.

66. A purification method for purifying one or more antibodies, which comprises providing a source material comprising one or more antibodies, tangential-flow filtering said source material with at least one semipermeable filter, wherein one or more of the source material, the semipermeable filter, or one or more solutions used in said tangential flow filtering comprises one or more amorphous polymer particles, and wherein the one or more polymer particles comprise a ligand capable of binding an antibody, and recovering the antibody.

67. A composition, membrane, filter, or filter apparatus for use in tangential flow filtration, wherein the composition, membrane, filter, or filter apparatus comprises one or more amorphous polymer particles comprising
i. a biopolymer selected from a polyester, a polythioester or a polyhydroxyalkanoate; or
ii. a polymer particle-forming polypeptide; or
iii. a polymer particle-binding polypeptide;
iv. a polypeptide fusion partner;
v. an affinity ligand;
vi. an enzyme;
vii. a fusion polypeptide comprising two or more of the above; or
viii. any combination of any two or more of (i) to (vii) above.

68. A polymer particle comprising
i. a biopolymer selected from a polyester, a polythioester or a polyhydroxyalkanoate; or
ii. a polymer particle-forming polypeptide; or
iii. a polymer particle-binding polypeptide;
iv. a polypeptide fusion partner;
v. an affinity ligand;
vi. an enzyme;
vii. a fusion polypeptide comprising two or more of the above; or
viii. any combination of any two or more of (i) to (vii) above;
wherein one or more of the polypeptides is or comprises the GB1 domain of protein G from Streptococcus spp.

69. A fusion polypeptide comprising a polymer particle-forming polypeptide and one or more GB1 domains of protein G from Streptococcus spp.

70. The polymer particle of claim 68 wherein the polymer particle has an immunoglobulin binding capacity of greater than 30 mg immunoglobulin/g wet polymer particle.

71. The method of claim 53 wherein the source material is selected from the group comprising
a source material that is or is derived from a cell lysate, or
a source material that is or is derived from a protein expression system; or is or is derived from a food, a dairy product or dairy processing stream, or a fermentate; or
a source material that is a solution, a reaction solution, a chemical synthesis solution, or a chemical synthesis intermediate.

72. The method of claim 53 wherein the polymer particle comprises polyhydroxyalkanoate.
